(19) United States
(12) Patent Application Publication
(10) Pub. No.: US 2024/0228532 A1
(43) Pub. Date: Jul. 11, 2024

(54) Peptide Sequences And Compositions At Air-Aqueous Interfaces For Lanthanide Recovery

(71) Applicants: The Trustee of the University of Pennsylvania, Philadelphia, PA (US); The City College of New York, New York, NY (US); University of Illinois Chicago, Chicago, IL (US); Northwestern University, Evanston, IL (US)

(72) Inventors: Kathleen J. Stebe, Penn Valley, PA (US); Daeyeon Lee, Wynnewood, PA (US); Ravi Radhakrishnan, Berwyn, PA (US); César de la Fuente-Nunez, Philadelphia, PA (US); Ivan Julian Dmochowski, Philadelphia, PA (US); E. James Petersson, Wynnewood, PA (US); Jason G. Marmorstein, Philadelphia, PA (US); Stephen Crane, Clarksburg, NJ (US); Eshe Jael Hummingbird, Philadelphia, PA (US); Yiming Wang, Philadelphia, PA (US); Elizabeth J. Biddinger, Englewood, NJ (US); Charles Maldarelli, Wayne, NJ (US); Robert J. Messinger, New York, NY (US); Raymond S. Tu, Forest Hills, NY (US); Luis Ortuno Macias, Bayville, NY (US); Surabh S. KT, New York, NY (US); Mark L. Schlossman, Chicago, IL (US); Bikash Sapkota, Chicago, IL (US); Pan Sun, Oak Ridge, TN (US); Monica Olvera de la Cruz, Wilmette, IL (US); Felipe Jiménez-Ángeles, Wilmette, IL (US); Baofu Qiao, Syossett, NY (US)

(21) Appl. No.: 18/448,427

(22) Filed: Aug. 11, 2023

Related U.S. Application Data
(60) Provisional application No. 63/400,695, filed on Aug. 24, 2022.
(60) Provisional application No. 63/371,080, filed on Aug. 11, 2022.

(51) Int. Cl. C07K 1/32

(52) U.S. Cl. CPC C07K 1/32

(57) ABSTRACT

Provided is, inter alia, a method, comprising: complexing a peptide-comprising surfactant (PEPS) to a rare earth element (REE) cation in a solution so as to form a PEPS-REE complex, the PEPS comprising a REE-binding region that preferentially binds to one or more REEs; and optionally recovering the REE cation from an air-liquid interface.

Also provided is a composition, comprising: a complex comprising (i) a peptide-comprising surfactant (PEPS), the PEPS comprising at least one lanthanide binding tag (LBT), the at least one LBT comprising one or more residues arranged in a binding region that coordinates with an REE cation so as to form a PEPS-REE cation complex, and (ii) an REE cation, the REE cation of the complex coordinated with the binding region of the PEPS.

Further provided is a method, comprising application of at least one of molecular dynamics, artificial intelligence, and a genetic algorithm to design a PEPS that comprises an LBT and preferentially binds one REE over at least one other REE.

### CROSS-REFERENCE TO RELATED APPLICATIONS

The present application claims priority to and the benefit of U.S. patent application No. 63/371,080, “Peptide Sequences At Air-Aqueous Interfaces For Lanthanide Recovery” (filed Aug. 11, 2022) and of U.S. patent application No. 63/400,695, “Peptide Sequences and Compositions At Air-Aqueous Interfaces For Lanthanide Recovery” (filed Aug. 24, 2022) All foregoing applications are incorporated herein by reference in their entireties for any and all purposes.

### GOVERNMENT RIGHTS

This invention was made with government support under DE-SC0022240 awarded by the Department of Energy. The government has certain rights in the invention.

### TECHNICAL FIELD

The present disclosure relates to the field of separations, in particular the field of rare earth element (REE) separations.

### BACKGROUND

The present disclosure addresses a new, ecofriendly, bioinspired and energy efficient method for the separation of Rare Earth Elements (REEs) in aqueous feedstocks from mining extraction and on-site processing. REEs are the heavy lanthanide metals in addition to yttrium and scandium. Their unique luminescent, magnetic, and catalytic properties have made them essential to the US economy, with applications ranging from catalysis to luminescent materials in displays, and that include a number of clean energy (rechargeable batteries, wind turbines, solar panels and hybrid vehicles) and other (e.g., satellites, magnets, and guidance systems) technologies.

Although the United States once dominated REE mining and production, environmental restrictions on current separation methods have limited domestic production. The development of robust domestic supply chains is of paramount importance. Accordingly, there is a long-felt need in the field for improved methods of separating and recovering REEs.

### SUMMARY

This disclosure provides, inter alia, new, green, separation technologies to contribute to the rebuilding of the domestic supply chain. Such technologies can include, e.g., novel peptide-comprising surfactants.

As is known, new separation methods are urgently needed to support an uninterrupted supply of Rare Earth Elements (REEs) used as essential components in advanced technologies important to the national economy. This disclosure provides, e.g., a bioinspired, green REE separation process based on peptide surfactants (PEPS) at air-water interfaces.

PEPS can include a hydrophilic amino acid sequence that selectively binds an REE cation and a hydrophobic sequence that confers surface activity. PEPS added to REE feedstocks can thus associate selectively with REE cations and adsorb to air-water interfaces for recovery in a froth flotation process that obviates the need for solvents as in solvent extraction methods.

The disclosed PEPS design strategy can use lanthanide binding tags (LBTs). LBTs are short peptide sequences that bind an REE and retain that bound state in complex chemical environments in large proteins. The binding loop domain influences the strength and selectivity of REE binding, and can be used to recover a particular REE over another competing REE or even over all competing REEs. As but one example, one can achieve optimal binding of a particular REE, e.g., Tb³⁺, to exploit its fluorescence properties in protein metallochemistry.

The disclosed technology can be used to, e.g., separate a given REE from one or more other REEs that are comparatively close in size to the given REE. The disclosed technology thus allows for PEPS that can select among REEs by controlling the peptide sequence and peptide selections (of the PEPS) and to enhance the peptides' amphiphilicity to promote their adsorption. This can be enhanced by, inter alia, machine learning-guided mutations, rationally-guided mutations, and by other processes.

The disclosed technology provides peptide constructs that can select among REEs by controlling the constructs' peptide sequence and by tailoring the peptides' amphiphilicity to promote adsorption. The constructs' design can be enhanced by, e.g., machine learning-guided mutations, rationally-guided mutations, and by other processes. Peptides are also amenable to inexpensive, scalable production using genetically engineered bacteria in biotechnological processes. For these reasons, this disclosure utilizes LBTs, as one can make changes to the LBTs' structures to design PEPS.

The presently disclosed technology provides a number of advantageous features, and some of these features are provided below.

Feature 1: PEPS that are surface active in the complexed state and bind REEs over divalent cations, e.g., like those in REE feedstock solutions. One can accomplish this objective by, e.g., addition of hydrophobes to the C-terminus of the LBT sequence. One can also utilize a co-surfactant system to promote synergistic PEPS adsorption. Cosurfactants can be designed to avoid attraction of REE to the interface; to promote the adsorption of the complex; and also to protect the binding loop in the vicinity of the interface. Co-surfactants can also be designed to stabilize foams formed by the disclosed compositions.

Feature 2: PEPS with enhanced selectivity among REEs. The binding loop domain already exhibits selectivity among the larger, lighter lanthanides. One can exploit a strategy based on substitutions at a particular site in the LBT binding domain known to play role in LBT selectivity among REEs. One can also use a parallel synthesis approach to screen libraries for selectivities across the REEs.

Evaluation: One can characterize PEPS:REE complexes at air water interfaces, which characterization can be used to support PEPS design. At the air-water interface, PEPS:REE surface activity can be probed by pendant drop, XPS of sampled monolayers, and by fluorescence confocal microscopy. Microfluidics assays integrated with confocal fluorescence can identify co-surfactant formulations. The molecular composition and arrangement can be probed by X-ray interrogation using X-ray reflectivity, X-ray fluorescence near total reflection, and X-ray absorption spectroscopy. Experiments synergize with molecular simulation, including all-atom simulation to provide 3-D candidate PEPS-cation complex conformation, thermodynamic insights into complex stability, energy landscape for adsorption from bulk to interface. Coarse-grained simulations provide insights into interaction among PEPS-cation complexes on densely-covered interfaces with and without co-surfactants.

Impact: This disclosure presents high scientific and societal impact. The research focuses on detailed molecular understanding of PEPS:REE complexation at the highly anisotropic air-water interface. PEPS have cooperative interactions lacking in simpler surfactants used thus far in ion froth flotation processes, providing a combination of affinity and selectivity that is unattainable in those systems.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIG. 1A Binding free energy of LBT1 with Ln³⁺ series. The line is included to guide the reader and as a representation of a theoretical correlation. The inset table contains the affinity constants of the peptide with metals, and the inset figure is a snapshot of the final peptide-Tb³⁺ configuration from microsecond-MD simulations. FIG. 1B Binding free energy of LBT1-LLA with Ln³⁺ series. The line is included to guide the reader and as a representation of a theoretical correlation. The inset table contains the affinity constants of the peptide with metals, and inset figure is a snapshot of the final peptide-Tb³⁺ configuration from microsecond-MD simulations. FIG. 1C Binding free energy of LBT1-3 with Ln³⁺ series. The line is included to guide the reader and as a representation of a theoretical correlation. The inset table contains the affinity constants of the peptide with metals, and inset figure is a snapshot of the final peptide-Tb³⁺ configuration from microsecond-MD simulations. FIG. 1D Quasi-equilibrium surface tension values, measured at different bulk concentrations of LBT1, LBT1-LLA, and LBT1-3 in the absence and presence of TbCl₃ at a ratio LBT:Tb³⁺1:4. The inset figures are snapshots of the final peptide or peptide-Tb³⁺ configurations at the air-aqueous interface from microsecond-MD simulations.

FIGS. 2A-2F. XR measurements from adsorbed layers of LBT1/Tb³⁺, LBT1-LLA/Tb³⁺, and LBT1-3/Tb³⁺ at the air-aqueous interface. FIG. 2A Normalized reflectivity (R/R_{F}) as a function of Q_{Z} normal to the surface of LBT1/Tb³⁺ at different bulk concentrations. FIG. 2B Normalized reflectivity (R/R_{F}) as a function of Q_{Z} normal to the surface of LBT1-LLA/Tb³⁺ at different bulk concentrations. FIG. 2C Normalized reflectivity (R/R_{F}) as a function of Q_{Z} normal to the surface of LBT1-3/Tb³⁺ at different bulk concentrations. FIG. 2D EDP as a function of the distance z normal to the surface of LBT1/Tb³⁺ at different bulk concentrations. FIG. 2E EDP as a function of the distance z normal to the surface of LBT1-LLA/Tb³⁺ at different concentrations. FIG. 2F EDP as a function of the distance z normal to the surface of LBT1-3/Tb³⁺ at different concentrations.

FIGS. 3A-3E FIG. 3A Interfacial concentration of LBT1, LBT1-LLA, and LBT1-3 as a function of peptide bulk concentration in the presence of Tb³⁺ cations at a ratio LBT:Tb³⁺1:4. FIG. 3B Terbium fluorescence intensity as a function of Q_{Z} near critical angle for total reflection for different concentrations of peptide and cation. FIG. 3C Terbium interfacial density as a function of peptide and cation concentration for ratio LBT:Tb³⁺1:4. FIG. 3D Number of Tb³⁺ per LBT peptide at the interfacial zone for different bulk concentrations of peptide and Tb³⁺ cations at a ratio LBT:Tb³⁺1:4. FIG. 3E Free energy (ΔΔG=ΔGᵢₙₜ−ΔG_{bulk}) of adsorption of peptides to the air-aqueous interface ΔGᵢₙₜ from a bulk aqueous solution ΔG_{bulk}. The blue bars represent the peptides forming a complex with Tb³⁺, whereas the green bars are for ion-free peptides.

FIGS. 4A-4D: FIG. 4A Interfacial concentration of LBT1-LLA and Tb³⁺ cations from bulk concentrations of 100 μM peptide and different bulk concentrations of Tb³⁺. FIG. 4B Number of Tb³⁺ per LBT1-LLA peptide from bulk concentrations of 100 μM peptide and different bulk concentrations of Tb³⁺. FIG. 4C Interfacial concentration of LBT1-3 and Tb³⁺ cations from bulk concentrations of 100 μM peptide and different bulk concentrations of Tb³⁺. FIG. 4D Number of Tb³⁺ per LBT1-3 peptide from bulk concentrations of 100 μM peptide and different bulk concentrations of Tb³⁺.

FIGS. 5A-5G: Tb³⁺ adsorption at the air-aqueous interface from molecular dynamics simulations. FIG. 5A Simulation box employed to simulate air-aqueous interfaces. The aqueous phase contains the peptides (gray ribbons), Tb³⁺ (magenta), Na (yellow), and Cl⁻ (green) ions, and the buffer MES (blue). Water is represented as a light-blue transparent medium. At t=0, the peptides forming complexes with Tb³⁺ are placed near the interface, whereas free Tb³⁺ ions are randomly placed in the aqueous phase. The size of the simulation box is shortened here. FIG. 5B Number of adsorbed Tb³⁺ ions per peptide molecules at the interface for systems of LBT1, LBT1-LLA, or LBT1-3 as a function of the surface concentration of peptide. The number of peptides per unit area is taken from example measurements. Top view of instantaneous snapshots showing FIG. 5C LBT1, FIG. 5D LBT1-LLA, and FIG. 5E LBT1-3 peptides at the interface. We use a transparent representation for some of the adsorbed peptides and Tb³⁺ ions to aid visualization. The coordination number of the free Tb⁺³ ions with oxygen atoms from the COO⁻ groups in the D11 and C-terminus groups of FIG. 5F LBT1 and FIG. 5G LBT1-LLA at the air-aqueous interface.

FIGS. 6A-6D. FIG. 6A Number of total Ln³⁺ per LBT1-LLA peptide at the interfacial zone for a fixed bulk concentration of peptide of 100 μM and different equimolar bulk concentrations of peptide and Ln³⁺ cations. FIG. 6B Number of total Ln³⁺ per LBT1-3 peptide at the interfacial zone for a fixed bulk concentration of peptide of 100 μM and different equimolar bulk concentrations of peptide and Ln³⁺ cations. FIG. 6C Γ_{Tb}_{³⁺}/Γ_{La}_{³⁺} for a fixed bulk concentration of LBT1-LLA peptide of 100 μM and different equimolar bulk concentrations of peptide and Ln³⁺ cations. FIG. 6D Γ_{Tb}_{³⁺}/Γ_{La}_{³⁺} for a fixed bulk concentration of LBT1-3 peptide of 100 μM and different equimolar bulk concentrations of peptide and Ln³⁺ cations.

FIGS. 7A-7C. Conformational free energy landscape of peptide-Tb³⁺ complexes computed from well-tempered metadynamics simulations. FIG. 7A LBT1/Tb³⁺ FIG. 7B LBT1-LLA/Tb³⁺, and FIG. 7C LBT1-3/Tb³⁺. Black dots represent the structures obtained from microsecond molecular dynamics simulations.

FIGS. 8A-8C. Free energy for different basins of peptide-Tb³⁺ complexes as a function of simulation time for FIG. 8A LBT1/Tb³⁺, FIG. 8B LBT1-LLA/Tb³⁺, and FIG. 8C LBT1-3/Tb³⁺.

FIGS. 9A-9F. Atomic distances as a function of time from the Tb³⁺ cation to the backbone oxygen of residues at positions 3 and 7, designated as N₃(O) and W₇(O), respectively. FIG. 9A LBT1 in bulk, FIG. 9B LBT1 at the air-aqueous interface, FIG. 9C LBT1-LLA in bulk, FIG. 9D LBT1-LLA at the air-aqueous interface, FIG. 9E LBT1-3 in bulk, FIG. 9F LBT1-3 at the air-aqueous interface.

FIGS. 10A-10B. Schematic illustration of the 2- and 3-slab model used to fit the reflectivity data. FIG. 10ALBT1 at the air-aqueous interface, FIG. 10B LBT1-LLA/Tb³⁺ complex at the air-aqueous interface.

FIGS. 11A-11B. Normalized fluoresce spectra from the samples containing Tb³⁺ cations at Q_{Z}=0.021 Å⁻¹. FIG. 11A Normalized fluorescence spectra from the reference sample containing 25 mM of Tb³⁺ at Q_{Z}=0.021 Å⁻¹. FIG. 11B Normalized fluorescence spectra from the samples containing 100 μM of Tb³⁺ at Q_{Z}=0.021 Å⁻¹.

FIG. 12. Integrated fluorescence intensity from Tb³⁺ L_{α} emission lines from the reference sample containing 25 mM of Tb³⁺.

FIGS. 13A-13B. Dynamic surface tension relaxation measurements for FIG. 13A solutions containing LBT1, LBT1-LLA, and LBT1-3 at different bulk concentrations, FIG. 13B solutions containing LBT1/Tb³⁺, LBT1-LLA/Tb³⁺, and LBT1-3/Tb³⁺ at different bulk concentrations.

FIGS. 14A-14F. XR measurements from adsorbed layers of LBT1, LBT1-LLA, and LBT1-3 at the air-aqueous interface. FIG. 14A Normalized reflectivity (R/R_{F}) as a function of Q_{Z} normal to the surface of LBT1 at different bulk concentrations, FIG. 14B Normalized reflectivity (R/R_{F}) as a function of Q_{Z} normal to the surface of LBT1-LLA at different bulk concentrations, FIG. 14C Normalized reflectivity (R/R_{F}) as a function of Q_{Z} normal to the surface of LBT1-3 at different bulk concentrations, FIG. 14D EDP as a function of the distance z normal to the surface of LBT1 at different bulk concentrations, FIG. 14E EDP as a function of the distance z normal to the surface of LBT1-LLA at different concentrations, FIG. 14F EDP as a function of the distance z normal to the surface of LBT1-3 at different concentrations.

FIG. 15. A) Interfacial concentration of LBT1, LBT1-LLA, and LBT1-3 as a function of peptide bulk concentration with no cations in solution.

FIGS. 16A-16F. Density distribution profile. FIG. 16A LBT1/Tb³⁺, FIG. 16B LBT1-LLA/Tb³⁺, and FIG. 16C LBT1-3/Tb³⁺, show the profiles at the interfacial zone (NH³⁺ and COO⁻ correspond to the charged ends from N and C terminal, respectively). FIG. 16D LBT1/Tb³⁺, FIG. 16E LBT1-LLA/Tb³⁺, and FIG. 16F LBT1-3/Tb³⁺, show the profiles from the interfacial zone to bulk solution (up to 10 nm normal to the interface).

FIGS. 17A-17C. XR and XFNTR measurements from adsorbed layers of LBT1-LLA/Tb³⁺ at the air-aqueous interface at a fixed peptide bulk concentration of 100 μM and different bulk Tb³⁺ concentration. FIG. 17A Normalized reflectivity (R/R_{F}) as a function of Q_{Z} normal to the surface. FIG. 17B EDP as a function of the distance z normal to the surface. FIG. 17C Terbium fluorescence intensity as a function of Q_{Z} near critical angle for total reflection.

FIGS. 18A-18C. XR and XFNTR measurements from adsorbed layers of LBT1-3/Tb³⁺ at the air-aqueous interface at a fixed peptide bulk concentration of 100 μM and different bulk Tb³⁺ concentration. FIG. 18A Normalized reflectivity (R/R_{F}) as a function of Q_{Z} normal to the surface. FIG. 18B EDP as a function of the distance z normal to the surface. FIG. 18C Terbium fluorescence intensity as a function of Q_{Z} near critical angle for total reflection.

FIGS. 19A-19D. FIG. 19A Interfacial concentration of LBT1-LLA, LBT1-3, and La³⁺ cations from solutions containing 100 μM of peptide and 100 μM of La³⁺ cations. FIG. 19B Number of La³⁺ per LBT1-LLA, and LBT1-3 peptides at the interfacial zone for a bulk concentration of peptide of 100 μM and 100 μM of La³⁺ cations. FIG. 19C Interfacial concentration of LBT1-LLA, Tb³⁺, and La³⁺ for a fixed bulk concentration of peptide of 100 μM and different equimolar bulk concentrations of Ln³⁺ cations. FIG. 19D Interfacial concentration of LBT1-3, Tb³⁺, and La³⁺ for a fixed bulk concentration of peptide of 100 μM and different equimolar bulk concentrations of Ln³⁺ cations.

FIGS. 20A-20C. XR and XFNTR measurements from adsorbed layers of LBT1-LLA/La³⁺ and LBT1-3/La³⁺ at the air-aqueous interface at bulk concentration of 100 μM of peptide and 400 μM of La³⁺. FIG. 20A Normalized reflectivity (R/R_{F}) as a function of Q_{Z} normal to the surface. FIG. 20B EDP as a function of the distance z normal to the surface. FIG. 20C Lanthanum fluorescence intensity as a function of Q_{Z} near critical angle for total reflection.

FIGS. 21A-21D. XR and XFNTR measurements from adsorbed layers of LBT1-LLA/Tb³⁺/L³⁺ at the air-aqueous interface at a fixed peptide bulk concentration of 100 μM and different equimolar concentrations of Tb³⁺ and La³⁺. FIG. 21A Normalized reflectivity (R/R_{F}) as a function of Q_{Z} normal to the surface. FIG. 21B EDP as a function of the distance z normal to the surface. FIG. 21C Terbium fluorescence intensity as a function of Q_{Z} near critical angle for total reflection. FIG. 21D Lanthanum fluorescence intensity as a function of Q_{Z} near critical angle for total reflection. Legend as follows, green: Tb³⁺=L³⁺=25 μM, orange: Tb³⁺=La³⁺=50 μM, yellow: Tb³⁺=La³⁺=100 μM, purple: Tb³⁺=La³⁺=150 μM, pink: Tb³⁺=La³⁺=200 μM, blue: Tb³⁺=La³⁺=350 μM, red: Tb³⁺=La³⁺=500 μM.

FIGS. 22A-22D. XR and XFNTR measurements from adsorbed layers of LBT1-3/Tb³⁺ La³⁺ at the air-aqueous interface at a fixed peptide bulk concentration of 100 μM and different equimolar concentrations of Tb³⁺ and La³⁺. FIG. 22A Normalized reflectivity (R/R_{F}) as a function of Q_{Z} normal to the surface. FIG. 22B EDP as a function of the distance z normal to the surface. FIG. 22C Terbium fluorescence intensity as a function of Q_{Z} near critical angle for total reflection. FIG. 22D Lanthanum fluorescence intensity as a function of Q_{Z} near critical angle for total reflection. Legend as follows, green: Tb³⁺=La³⁺=25 μM, orange: Tb³⁺=La³⁺=50 μM, yellow: Tb³⁺=La³⁺=100 μM, purple: Tb³⁺ La³⁺⁼¹⁵⁰ μM, pink: Tb³⁺=La³⁺=200 μM, blue: Tb³⁺=La³⁺=350 μM, red: Tb³⁺=La³⁺=500 μM.

FIG. 23 provides the structure of exemplary peptide LBT1.

FIG. 24 provides the structure of exemplary peptide LBT1-5, which peptide has a net charge of −5 in the unbound state.

FIG. 25 provides exemplary characterizations of three variants of the PEPS sequence to preserve the binding selectivity among the lanthanides by amending non-binding ligands outside and inside of the loop.

FIG. 26 provides an exemplary depiction of the disclosed technology, showing LBT coordination with a REE cation. The PEPS adsorbs to a bubble surface (for example, the surface of a bubble that has passed through the solution that includes the PEPS and the REEs). The resultant foam can be collected, and then the REEs can be recovered from the foam.

FIG. 27 provides exemplary results showing the surface tension (at air-liquid interface) exhibited by complexation of various PEPS with Tb³⁺ cations.

FIG. 28. Exemplary results showing that PEPS carry Tb³⁺ to the air-liquid interface.

FIG. 29. Exemplary, non-limiting results showing the ratio of cation to PEPS in an exemplary, non-limiting embodiment.

FIG. 30. Illustrates the presence of an interfacial PEPS-REE layer that is thicker than a monolayer.

FIG. 31. Provides a non-limiting illustration of PEPS-REE complexes at the air-liquid interface, including PEPS chelation of an REE that is not at the REE-binding region of the PEPS.

FIG. 32. Provides a further non-limiting illustration of PEPS-REE complexes at the air-liquid interface, including PEPS chelation of an REE that is not at the REE-binding region of the PEPS.

FIG. 33: Provides (left) an image of a PEPS-containing solution with no REE cation present and (right) an image of a PEPS-containing solution with 400 μM Tb cation present; upon withdrawal of fluid from the right-hand drop, the drop forms a bag-like structure, which bag-like structure indicates the formation of an interfacial film (sometimes termed a “skin”) at the air-liquid interface. The film can be viscous; the film can also be elastic.

FIG. 34. Provides a study of the “clean” air-water interface. There was a very small dissipation in the interface at the “clean” air-water interface, and the flow field shows signatures of 2-D incompressibility.

FIG. 35. Provides a study of a system having 100 μM LBT1LLA absent Tb³⁺ at air-buffer interface, with a buffer comprising NaCl 100 mM and MES (2-(N-morpholino)ethanesulfonic acid) 50 mM in water. As seen, Bq-4, and dissipation in interface is significant. This is similar to the buffer alone, suggesting that MES generates a viscous layer.

FIG. 36. Provides a study of a system having 100 μM LBT1LLA with 25 μM Tb³⁺ at air-buffer interface. As shown, Bq-230; dissipation is dominated by the interface and is significant.

FIG. 37. Provides a study of a system having 100 μM LBT1LLA with 100 μM Tb³⁺ at air-buffer interface. The data suggested the presence of slabs of elastic film moving in different directions.

FIG. 38. Provides a study of a system having 100 μM LBT1LLA with 400 μM Tb³⁺ at air-buffer interface. The data suggest that the peptides and Tb cation have formed a highly elastic film at the air-aqueous interface. This is consistent with the hypothesis of two populations of Tb cations at the interface, with one population of Tb cation in the binding loop, and the other interacting with the peptide outside of the binding loop, promoting the formation of a network.

FIG. 39. Provides an example depiction of a PEPS-REE complex, as shown, LBTLLA can form an elastic film laden with multivalent cations. As shown, the surface is enriched with lanthanides over monovalent cations, and the film can stabilize bubbles in ion foam fractionation. As shown here, at low Tb cation concentrations, the film is a viscous fluid film. At higher Tb cation concentration, the film shows evidence of domains of high shear modulus that cause the microparticles at the interface to move in a highly correlated manner. When the Tb cation is present is excess of the peptide, the film is highly elastic and (without being bound to any particular theory or embodiment) apparently homogeneous. The presence of elastic films or skins can, in some cases, depend on excess charge and/or chelating cations.

FIG. 40. Illustrates that an LBT can be selective for LBT is selective for Tb³⁺ over La³⁺ in bulk based on K_{D}.

FIG. 41. Provides an illustration of peptides at an air-liquid interface binding either Tb³⁺ or La³⁺.

FIG. 42. LBTs form interfacial films that bind Tb³⁺ over La³⁺ from mixed solutions. The ratio of surface concentrations of Tb³⁺ and La³⁺ as determined by X-ray Fluorescence Near Total Reflection (XFNTR) is shown at interfaces of peptide solutions exposed to equimolar mixtures of the La³⁺ and Tb³⁺. These data show that the interface can bind cations selectively, favoring La³⁺ at dilute cation concentrations and Tb³⁺ at elevated cation concentrations.

FIG. 43. Provides a summary of certain aspects of the disclosed technology. As shown, LBT peptide surfactants adsorb at air-water interfaces, bring lanthanide cations to the interface, and interact with more than one lanthanide per peptide. Lanthanide chelation promotes the formation of highly elastic layer, and the interfacial layer is selective for Tb³⁺ for mixed equimolar solutions of La³⁺ and Tb³⁺. Selectivity can be attenuated compared to that in bulk, which can (without being bound to any particular theory) be attributed to residual charge on the bound peptide.

FIGS. 44A-44D. FIG. 44A Configuration of LBT1-Ln3+ binding complex for MD simulation. FIG. 44B Schematic of the deep neural network (DNN). FIG. 44C and FIG. 44D Schematics of a simplified genetic algorithm for LBT1 mutant selection process.

FIGS. 45A-45B. Plot of binding affinity AG for LBT1 mutants against ten different lanthanide cations from FIG. 45A ML predictions and FIG. 45B fluorescent measurements.

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” can include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints. The endpoints of the ranges and any values disclosed herein are not limited to the precise range or value; they are sufficiently imprecise to include values approximating these ranges and/or values.

As used herein, approximating language can be applied to modify any quantitative representation that can vary without resulting in a change in the basic function to which it is related. Accordingly, a value modified by a term or terms, such as “about” and “substantially,” may not be limited to the precise value specified, in some cases. In at least some instances, the approximating language can correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” can refer to plus or minus 10% of the indicated number. For example, “about 10%” can indicate a range of 9% to 11%, and “about 1” can mean from 0.9-1.1. Other meanings of “about” can be apparent from the context, such as rounding off, so, for example “about 1” can also mean from 0.5 to 1.4. Further, the term “comprising” should be understood as having its open-ended meaning of “including,” but the term also includes the closed meaning of the term “consisting.” For example, a composition that comprises components A and B can be a composition that includes A, B, and other components, but can also be a composition made of A and B only. Any documents cited herein are incorporated by reference in their entireties for any and all purposes.

### Exemplary Disclosure

The present disclosure concerns, inter alia, peptide surfactants (PEPS) that complex selectively to REE cations in a solution. One can exploit their adsorption to rising air bubbles sparged through the solution. The REEs can be collected in the enriched foam formed from these bubbles at the top of the solution. The use of ecofriendly materials contrasts to the current practice of REE separation from the feedstocks into organic solvents, and low energy costs are associated with foam fractionation and gas sparging relative to solvent extraction, which requires multiple liquid-liquid extraction cycles.

One unique feature of this separation are the PEPS, surface active peptides designed to bind selectively with particular REEs. This disclosure describes examples of these molecules and illustrates the molecular details of their complexation with REEs and their adsorption in the complex environment of a fluid interface. PEPS can be based on lanthanide binding tags (LBTs), peptide sequences with hydrophilic residues arranged as a binding loop that forms a coordinating sphere of ligands around the REE cation. The peptide sequences in LBTs can be derived from the calcium binding loops present in natural calcium binding proteins, and LBT candidates can be, e.g., optimized to bind REEs that demonstrate strong fluorescence, terbium and europium. Further hydrophobic peptide sequences can be appended to the loop to make the LBT amphiphilic. By changing the amino acid sequence of the peptide loops, the affinity to a particular lanthanide cation can be tuned for selective separation. These peptides are useable in inexpensive, scalable production using genetically engineered bacteria in biotechnological processes.

Described herein are PEPS-REE complexes, which complexes can be present as aggregates and/or as films. The complexes can be present in a form that is more than one monolayer in thickness.

One can use peptides known to bind to a particular REE to develop PEPS. One can quantify binding and aggregation in water, and will determine PEPS' ability to adsorb and complex with REE cations at the interface. One can also amend the sequence of amino acids in the binding loop to achieve selectivity in REE complexation. Particular positions in the loop can dictate selectivity, and this concept can be explored using the molecular models and simulations developed for the high selectivity LBTs. One can also, as described elsewhere herein, incorporate a comparatively hydrophobic moiety at an end of the PEPS. The hydrophobes can be at either end of the PEPS' linear sequence, with the binding loop between them. When the peptide binding loop wraps the REE, the hydrophobic ends can form a hydrophobic domain. For this reason, one can add hydrophobes to either end of the linear sequence so as to promote their hydrophobicity.

Without being bound to any particular theory or embodiment, at least one other location in the binding loop can contribute to the hydrophobic character of the PEPS. In one example given herein, W (tryptophan) is located at position 7 in the current binding loop. This position can be changed (e.g., in mutants), and one can substitute other moieties (e.g., the non-natural amino acid acridone) at this and other locations. In this way, one can tune the hydrophobicity of the PEPS by selection of the amino acid present in the binding loop.

This disclosure presents both high scientific and high societal impact, as PEPS provide a combination of strong binding and selectivity unattainable in current surfactant REE complexes. Further this disclosure provides methods for REE separation with significant economic impact.

### Machine Learning

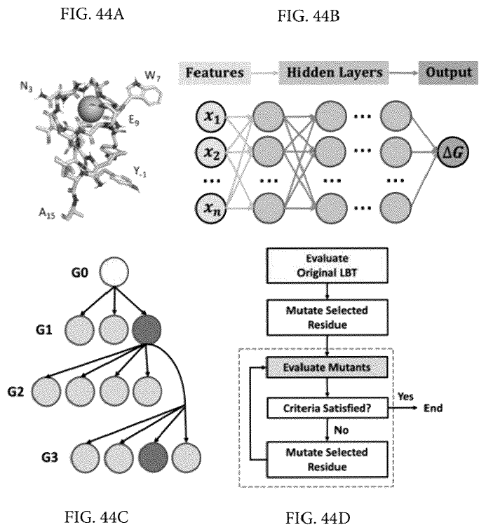

One can apply, for example, combined molecular dynamics (MD) simulation, machine learning (ML) and optimization methods for computational design of LBT peptides with improved lanthanide binding selectivity. A non-limiting computational peptide design scheme can involve the following aspects:
(1). Perform all-atom molecular dynamics with enhanced sampling techniques (metadynamics) of LBT-Ln3+ binding complex. Calculate key physiochemical quantities and thermodynamic properties from (conformational free energy landscape, binding coordination, hydration properties) from a molecular simulation.
(2). Train a supervised learning model (Deep Neural Network) to build quantitative predictive correlation between MD simulation features and experimental binding affinity (AG).
(3). Apply genetic algorithm and ML predictions to achieve high-throughput screening for LBT mutants. See, e.g., FIG. 44.

Regular MD simulations were performed for each of the 30 systems including LBT1, LBT1 mutant and LBT3 with 10 different lanthanides. Ten input features include van der Waal radius and Lennard-Jones potential depth of lanthanide, solvent accessible surface area of LBT peptide, the mean distances between the trivalent lanthanide cation and oxygen atoms from the six coordinated residues of the LBT peptide and water obtained from simulations. Metadynamics simulations are then performed for LBT3 with three selected lanthanides to obtain the free energy difference between six different class of metastable binding scenarios to expand the training data set. The total number of input data was ˜50 for ML training. The knock-one-out cross validation was applied during the ML training.

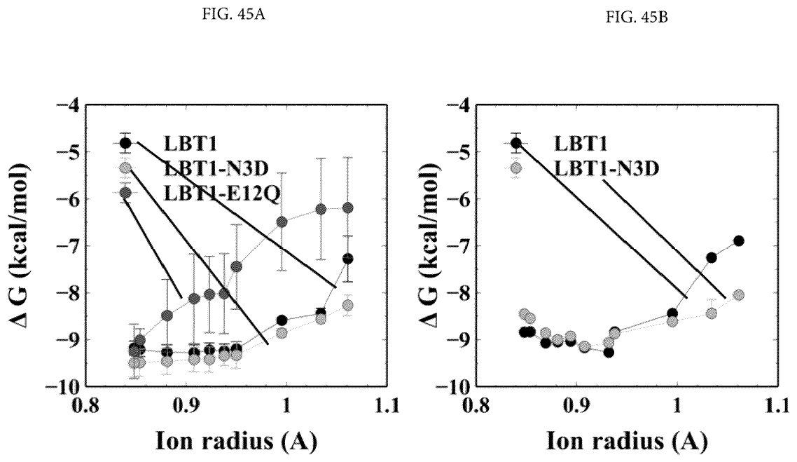

As shown in FIG. 45, the ML prediction and experimental measurements of binding affinity for LBT1 wildtype and the two single mutants are consistent with each other across two different sequences, each binding to 10 different ions. The ML model generates a reliable AG prediction with input data generated from metadynamics simulations of LBT-Ln3+ systems.

The overall effect of mutant of LBT on lanthanides binding can be described by the so called “affinity-selectivity trade-off”. Upon replacing polar residue (N) with negatively charged residue (D) at position 3, binding affinity for light lanthanides (with radius larger than 0.9 Å) increased but the overall binding specificity is decreased. However, upon replacing negatively charged residue (E) with polar residue (Q) at position 12, the LBT mutant is predicted to have an improved binding selectivity against ten different lanthanides but with decrease in overall binding affinity.

### Additional Disclosure

Solvent extraction is widely used for the separation of rare earth element trivalent cations (REEs or Ln³⁺) from an aqueous phase into an organic solvent. This process is energy intensive and environmentally unfriendly, requiring large volume of organic solvents and organo-phosphate surfactants. Here, we exploit the high affinity of a surface-active Lanthanide Binding Tag (LBT) peptide (LBT1, YIDTNNDGWYEGDELLA), that coordinates selectively with Ln³⁺ ions for its use in bioinspired/eco-friendly extraction processes in which the complexed LBT/Ln³⁺ peptide adsorbs to the air/aqueous interfaces of bubbles for foam recovery. To understand the surface activity and identify the bound cationic species at the air-aqueous interface, we characterized the surface molecular adsorption and arrangement of LBT1, the more surface active LBT1-LLA (YIDTNNDGWYEGDELLALLA), and the less negatively charged LBT1-3 (YIDTNNDGWYEGNELLA).

X-ray reflectivity (XR) and x-ray fluorescence near total reflection (XFNTR) measurements of the adsorbed layer were used to compute the surface concentration of the peptide and the Ln³⁺ cation, and electron density profile (EDP) of the interfacial layers.

The addition of three hydrophobic residues to LBT1 increased the adsorption of Tb³⁺ ions to the air-aqueous interface. We demonstrated that this cation adsorption enhancement was also promoted by electrostatic interactions between the charged LBT1-LLA/Tb³⁺ complexes and free trivalent ions in solution. Moreover, that bridging of complexes via O—Tb—O association can be eliminated by substituting negatively charged groups that do not participate in the selective coordination with neutral amino acids. LBT peptides can promote the adsorption of lanthanides selectively and this affinity can be controlled by varying the concentration of ions in solution. The ability to tune the amino acid sequence of these surface-active molecules to either improve their adsorption and/or selectivity with REEs allows for an advantageous, green, eco-friendly, and selective separation method of REEs.

Rare earth elements (REEs) are those elements between Lanthanum and Lutetium on the periodic table, also known as Lanthanides. Yttrium and Scandium are often included in this category of elements as they shared chemical and physical similarities with the group and exist in nature with other Lanthanides (Ln³⁺).

The unique properties of REEs make possible their use in a wide range of industrial applications, such as electronics, catalysis, clean energy, batteries, magnetics, and others. Deposits of these elements are found all over the world in the Earth's crust. The major ore deposits containing large amount of these elements are in China, the United States, Australia, India, and Russia.

Although the United States possesses domestic resources of REEs, the country is currently totally reliant on imports. In fact, in 2020 all the REEs consumed by the United States were imported, principally from China, Estonia, Japan, and Malaysia. The low mineable concentration of REE in ores makes it economically infeasible to use current technology for their extraction in the US. Moreover, environmental regulations imposed by the US government in the 1990s have promoted the reduction in local mining of these precious metals. While locating new geological deposits might increase the domestic supply of REEs, the development of new eco-friendly extraction and recycling technologies will certainly increase the local production of these elements by permitting the use of existing sources of REEs.

Before the separation of REEs from a concentrated solution, ores go through a series of physical separation and hydrometallurgical processing. Deposits or ores containing REEs are distinctive from each other. Therefore, the technologies and mineral processing implemented for obtaining concentrated solutions of REEs are broad and depend on the type of ore or mineral that is being processed. Ore beneficiation, mineral concentrate decomposition, and rare earth leaching are techniques that are commonly used for the preconcentrating of REEs solutions. Likewise, several chemical separation techniques can be used for the separation of REEs from concentrated solutions containing these elements, such as ion exchange, chromatography, solvent extraction processes, and others. The solvent extraction process takes advantage of the ability of metal ions to be transported across the interface between an aqueous solution and a non-miscible organic solution.

Generally, the aqueous solution contains the valued ions and soluble impurities, while the organic solution contains an extractant. The desired solute is initially dissolved in the aqueous solution, but eventually is distributed between the two phases until equilibrium is achieved. The time of contact or mixing of the two solutions depends on the affinity of the solutes for the two solvents, and after the two phases are separated the liquid loaded with the valuable solute is called “extract”, while the liquid containing only undesirable solutes and impurities is called “raffinate”.

Separation and purification of REEs have relied upon solvent extraction because of the extensive advantages of the process, such as simple fast and continuous operation, mild process conditions, and inexpensive handling of large quantities of materials. Solvent extraction process for the separation of REEs requires an abundance of solvents. Due to the high viscosity of the extractants use for the collection or REEs, solvents like kerosene and certain aromatics are used to dissolve these extracting components.

Although the different techniques employed throughout the REEs separation and purification process—from mining to solvent extraction—have the potential for environmental damage when not controlled and managed properly, the environment impact is mainly inherited from the solvent extraction stage.

For REEs separation and purification from bastnaesite, solvent extraction contributes approximately 75% to the terrestrial acidification; more than 60% to the global warming potential; more that 50% to the terrestrial, freshwater, and marine eutrophication; and around 70% to the resource depletion for water on the entire REEs extraction process that is represented by mining, beneficiation, acid roasting, leaching, and solvent extraction.

Ion foam flotation (IFF) is a promising eco-friendly approach for the extraction and purification of REEs. IFF is a separation technique that involves adding soluble surfactants or extractants with opposite charges to those of the target ions into solution containing the desired elements to form an extractant-ion complex that will be collected by passing gas bubbles throughout the aqueous solution. To date, IFF has been mainly applied for adsorption techniques, industrial wastewater treatment, and recovery of precious metals. Recently, IFF has received attention for its application in the separation and purification of REEs due to its simplicity, low energy requirements, rapid operation, and eco-friendly approach. However, the use of this technique for the separation and purification of REEs has been considered as an intrinsically immature technology and has not yet found applications at a large commercial scale.

IFF studies have investigated selective separation of REEs. They have demonstrated that the affinity for REEs is higher for higher atomic number elements over those with a smaller number. as well as a significant influence in the selectivity of REEs by the pH of the media and the presence of chloride ions in solution. For the surfactants used in these studies, the electrostatic attraction/coordination between the REEs and the extractants determine the selectivity towards the different trivalent cations, which poses an enormous challenge for simple surfactants with non-cooperative binding interactions.

Calcium-containing proteins have been explored to study their coordination with lanthanides ions based on the similarity of trivalent Ln³⁺ to divalent Ca²⁺ in ionic radii and metal coordination chemistry. Furthermore, researchers have engineered biomolecules that bind naturally with Ca²⁺ to enhance the affinity to Ln³⁺ ions and in some cases to increase the preference towards specific elements.

Lanthanide Binding Tags (LBTs) are short peptides (constituted by 15 to 20 amino acids) designed and engineered based on EF-hand motifs of calcium-binding proteins. LBT1, with the amino acid sequence YIDTNNDGWYEGDELLA, is a linear peptide of 17 amino acids that has been optimized by screening methods to coordinate selectively with Ln³⁺ ions. This peptide possesses a higher affinity for Tb³⁺ compared to the rest of the trivalent lanthanides. LBT1 is composed of hydrophilic amino acids sequences that selectively bind Ln³⁺ ions, and hydrophobic amino acids located at the end of the C-terminus, which form a hydrophobic cluster that is considered to increase the Ln³⁺ selectivity.

In this discussion, we explore the affinity of this peptide with Tb³⁺ ions as well as the hydrophobicity of the molecule for a bio-inspired, green REEs separation process at air-aqueous interface. Furthermore, we enhanced the hydrophobicity of LBT1 by extending the C terminus of the peptide with the addition of three hydrophobic residues (LLA) to improve the adsorption and extraction of REEs. Without being bound to any particular theory, one can hypothesize that the addition of these three residues to the backbone of the peptide will not affect its coordination and selectivity with Ln³⁺ ions. Likewise, we expect that the hydrophobic residues attached to the C-terminus of both LBT1 and LBT1-LLA, which do not participate in the coordination loop will promote the adsorption of the peptides to the air-aqueous interface without compromising the cation complexation.

The disclosed separation process can include several steps. Initially, peptides added to the ionic feedstocks coordinate selectively with REEs cations. Next, adsorption of complexes containing REEs to air-aqueous interfaces of bubbles will occur. This process can be initiated by introducing gas bubbles through the feedstock. Finally, LBTs-REEs complexes will “float” to the top of the solution where they will be collected as a foam for REEs recovery.

This disclosure reviews the fundamentals on the adsorption of LBT peptides in the absence and presence of Ln³⁺ cations to address the second step in the proposed separation process. The surface activity of LBTs:REEs complexes is probed by pendant drop tensiometry (PDT) to confirm the adsorption of species to the air-aqueous interface. Detailed characterization of the interface molecular composition and arrangement is determined by X-ray interrogation of adsorbed complexes using X-ray reflectivity (XR), and X-ray fluorescence near total reflection (XFNTR).

### Materials and Methods

### Materials

LBT1: YIDTNNDGWYEGDELLA (purity 98%) and LBT1-LLA: YIDTNNDGWYEGDELLALLA (purity ≥95%) both labeled at the N-terminus with a free amine and labeled at the C-terminus with a free acid, and LBT1-3: YIDTNNDGWYEGNELLA (purity ≥95%) labeled at the N-terminus and at the C-terminus with a free amine, were purchased from GenScript (Piscataway, NJ, USA), diluted to a stock concentration of 150 μM in buffer solutions containing 100 mM of NaCl (purity ≥99.5%, Sigma-Aldrich) and 50 mM of MES (purity ≥99.5%, Sigma-Aldrich) at a pH of 6, and used without additional purification. Anhydrous TbCl₃ (purity ≥99%) was purchased from Sigma-Aldrich and diluted to a stock concentration of 25 mM in the same buffer solution containing 100 mM of NaCl and 50 mM of MES. Buffer solution is filtered using a 0.22 μm polytetrafluoroethylene filters. Ultrapure water is obtained from a Milli-Q water filtration unit (EMD Millipore) with a resistivity of 18.2 mΩ cm and used for preparation of buffer solution.

### Luminescence Spectroscopy

Fluorescence resonance energy transfer (FRET) between Tryptophan (Trp) and Tb³⁺ was monitored with a Tecan Spark plate reader, using a 96 well plate in triplicate. Trp was excited at 280 nm and emission spectra was detected at 545 nm. As the concentration of Tb³⁺ increases in solution for a constant concentration of peptide, the collected emission became more intense until the luminescence intensity became constant (maximum value for saturation of peptide).

The collected data was fitted to produce thermodynamic parameters for the association of the peptide with Tb³⁺. To obtain thermodynamic association parameters for the rest of the series of lanthanides, displacement assays were carried out. Tb³⁺ cations were added to the peptide solution, followed by increasing amounts of other Ln³⁺. As more of the other Ln³⁺ was added, Tb³⁺ was displaced. The other lanthanides do not emit at 545 nm, so this displacement promoted a reduction in detected luminescence, that allowed to determine binding free energy and affinity constants for the rest of the Ln³⁺ series one at the time.

### Pendant Drop Tensiometry

Dynamic surface tension was measured using a pendant drop tensiometer (Attension Theta, Biolin Scientific, Stockholm, Sweden). For this we used a drop profile of the different solutions of LBTs or LBTs:Tb³⁺ at different concentrations to indirectly measure surface tension. Drops with volume of 14 μL were formed and suspended from a 16-gauge metal needle attached to a volume control system. For each run drop images were captured over time until an approximately constant surface tension was achieved. The surface tension values were computed by fitting the experimental drop shape to a theoretical profile governed by the Laplace equation of capillarity.

### Liquid Surface X-Ray Measurements

X-ray reflectivity (XR) and x-ray fluorescence near total reflection (XFNTR) measurements were conducted at NSF's ChemMatCARs, sector 15-ID-C experimental hutch at the Advanced Photon Source in Argonne National Laboratory (Lemont, IL) using 10 keV x-rays.

The liquid sample was placed in a trough of maximum surface area of 21 cm² within the x-ray reflectivity apparatus at 15-ID-C. The trough was filled with approximately 6 mL of solutions of LBTs or LBTs:Tb³⁺. The trough was contained in a sealed aluminum box, resting on a vibration isolation table. After the trough was filled, the box was closed. A slight overpressure of helium was kept in the box to reduce x-ray background scattering. All measurements were taken after approximately 2 hours of the filling process and at room temperature.

### X-ray Reflectivity (XR)

X-ray reflectivity measures the interfacial electron density profile. X-ray reflectivity data were measured as a function of wave vector transfer Q_{Z}=(4π/λ)sin (α) along the surface normal to cover the range 0.016 Å⁻¹<Q_{Z}<0.6 Å⁻¹, where a is the angle of incidence and A is the x-ray wavelength λ=1.24 Å. XR data was measured with a Pilatus 200K area detector. Reflectivity was measured multiple times to check for stability. XR measurements were fit to a model functional form to compute the electron density ρ(z) along the z direction perpendicular to the interface, and averaged over the x-y plane of the surface, with the model function represented by a sum of error functions:

$$ρ ( z ) = \frac{1}{2} {\sum }_{i = 0}^{N - 1} erf ( \frac{z - {z}_{i}}{\sqrt{2} σ} ) ( {ρ}_{i + 1} - {ρ}_{i} ) + \frac{{ρ}_{0} + {ρ}_{N}}{2} \qquad ( 1 )$$

with erf(z)=(2/\sqrt{π})∫₀^{z}e^{-t}^{²}dt; N the number of internal interfaces within the surface film, a the interfacial roughness, ρ₀ the electron density of the bulk aqueous phase, ρ_{N} the electron density of the air phase, and ρ₁ and zᵢ the electron density and the position of the ith slab and interface, respectively. The thickness of the i^{th} slab, dᵢ, is defined as |z−zᵢ|. X-ray reflectivity was computed using the Parrat formalism by discretizing the electron density profile (EDP) from equation 1. XR measurements were normalized to the Fresnel reflectivity R_{F}, which is calculated for a theoretical liquid-air interface where the electron density varies as a step function between the two bulk phases.

### X-Ray Fluorescence Near Total Reflection (XFNTR)

XFNTR measures the surface number density of fluorescing metal ions (number per Δ²). X-ray fluorescence intensity measurements were measured for a range of Q_{Z} near the critical value Q_{c} for total reflection. The x-ray fluorescence spectrum was measured by a Vortex-90-EX silicon drift detector placed above the interface and spectra were normalized to the incident beam intensity. The XFNTR signal was obtained by integrating the fluorescence peak of Tb³⁺ over the acceptance volume in the aqueous phase.

### Molecular Dynamics (MD) Simulations

MD simulations are performed to modeling the LBT-Tb³⁺ binding complex in the aqueous solution using GROMACS package. Peptides were modelled using CHARMM36 force field. Terbium cation was modelled using the modified CHARMM force field, which were designed to match the hydration structure and hydrogen free energy from experimental measurements. Solvent was modelled using the modified Tip3p water model under neutral pH condition. Unless differently stated, we use periodic boundary conditions in the x-, y-, and z-directions. Sodium and chloride ions were used to neutralize the system and the concentration of NaCl was 0.1 M, which is comparable to the experimental conditions. Particle Mesh Ewald algorithm was adopted for the calculation of long-range electrostatic interactions. The integration time step was set to 2.0 fs, and the LINCS algorithm was employed to constrain the lengths of all chemical bonds involving hydrogen atoms at their equilibrium values. The solvated system was first energy minimized using the Steepest Descent (SD), while algorithms were used to remove unfavorable contacts. The isochoric isothermal (NVT) simulation were then performed at room temperature of 298K using a stochastic velocity rescaling algorithm for 5 ns. After the equilibration stage, isobaric isothermal (NPT) simulation were performed under room temperature and ambient pressure (1 bar) a using velocity rescaling thermostat and Parrinello-Rahman barostat.

### Well-Tempered Metadynamics Simulations

Well-tempered metadynamics simulations were performed to construct free energy landscape of the LBT1/Tb³⁺, LBT1-LLA/Tb³⁺, and LBT1-3/Tb³⁺ binding complex using the GROMACS package 2020.2 and Plumed 2.6.2 version. The initial configuration of the LBT1/Tb³⁺ binding complex was obtained from the X-ray measurements (PDB code: 1TJB). The initial configuration of LBT1-3/Tb³⁺ binding complex was obtained by residue mutation using the Scwrl4 program. The initial configuration of LBT1-LLA/Tb³⁺ binding complex was obtained by extending the extra -LLA motif from C-terminal of LBT1 peptide with extended conformation using Discovery Studio. The buffering distance of LBT1-3/Tb³⁺ complex was set to be 1.2 nm which results in a cubic box with length around 5 nm.

Metadynamics simulations of three systems were performed after using the final configurations obtained from a short NPT simulation. Here, we used two collective variables (CV). First, the coordination number (CN) of Tb³⁺ which refers to the number of oxygen atoms from the LBT peptide that have a separation distance with Tb³⁺ less than 0.27 nm which, do not include the oxygen of water. Second, the root mean square deviation of α carbon (Cu-RMSD) of the 1^{st}-12^{th} residues (corresponding to position 1 to 12) with respect to the reference structure, (PDB code: 1TJB). The height of the gaussian potential was 1.0 kJ/mol which was deposited every 500 steps (PACE). The widths (SIGMA) of gaussian potentials for CN and Ca-RMSD were 0.3 and 0.05, respectively. The bias factor for well-tempered metadynamics was set to be 3. In addition, the upper wall restraining potential was imposed at Cα-RMSD=0.6 nm and the lower wall restraining potential was imposed at CN=5. Both upper and lower wall potential used a force constant (KAPPA) of 300 kJ and a power (EXP) of 2. The grid boundaries (GRID_MIN and GRID MAX) for CN and Cu-RMSD were set to be 2-11 and 0-1.1 nm, respectively. The number of bins for every collective variable (GRID BIN) was set to be 500. Eight replicas with different random seeds were run in parallel with each replicate lasting around 2 μs.

### Air-Aqueous Interface MD Simulations

MD simulations at the air-aqueous interface of three short peptides known as lanthanide binding tags (LBT) were performed. The first peptide referred to as LBT1, is modeled using the structure 1TJB from the protein data bank, which consists of a complex formed by the peptide and a trivalent lanthanide cation. The second peptide LBT1-LLA is built by taking the structure of LBT1 and adding two leucine (L) amino acids and one alanine (A) amino acid. At the two ends, both peptides are terminated by the charged groups NH⁺₃ and COO⁻. The net charge of these two peptides is −5e. The third peptide LBT1-3 is built by taking the structure of LBT1 and mutating the aspartic acid (D) in position 11 by asparagine (N) such that D11 becomes N11. At the two ends, the peptide is terminated by the charged groups NH⁺₃ and CONH₂. The net charge of this peptide is −3e which is exactly compensated when it forms a complex with trivalent ions.

The configurations of the peptide molecules employed in the microsecond MD simulations are consistent with the well-tempered metadynamics simulations. To simulate the molecules in bulk, we employed a cubic simulation box of side L=8 nm. The simulation box contains one peptide molecule forming a complex with the ion Tb⁺³, 33 Na⁺ ions, 31 Cl⁻ ions, and 16780 water molecules, equivalent to a 0.1 M of NaCl. We used the same composition to simulate the complexes near an air-aqueous interface by extending the simulation box in the z-direction, namely, the simulation box dimensions are 8 nm, 8 nm, and 32 nm, in the x, y, and z directions respectively. We used periodic boundary conditions in the three directions. The systems containing a single molecule are simulated for at least 2 microseconds. To equilibrate the molecules in bulk and at the interface we performed microsecond MD simulations using a time-step of 2.5 fs at T=298 K. The bulk systems are simulated using the NPT ensemble while for the systems with an interface we use the NVT ensemble. The temperature is controlled using the Nose-Hoover thermostat, and the pressure is maintained using the Parrinello-Rahman barostat.

The adsorption of Tb³⁺ is simulated employing a simulation box of 10 nm by 10 nm by 40 nm, in the x, y, and z directions, respectively. The aqueous phase occupies about 16 nm in the z-direction of the simulation box, and we use periodic boundary conditions in the three directions. At t=0, the peptide-Tb³⁺ complexes are placed near the air-aqueous interface. The simulations are performed for one microsecond. Equilibration of the adsorption of cations is achieved at about 600 ns of the simulation. We use the last 400 ns of the simulation trajectory to calculate the average ions adsorption at the interface.

### Results and Discussion

Thermodynamic Study of LBTs Peptide with Lanthanide Cations

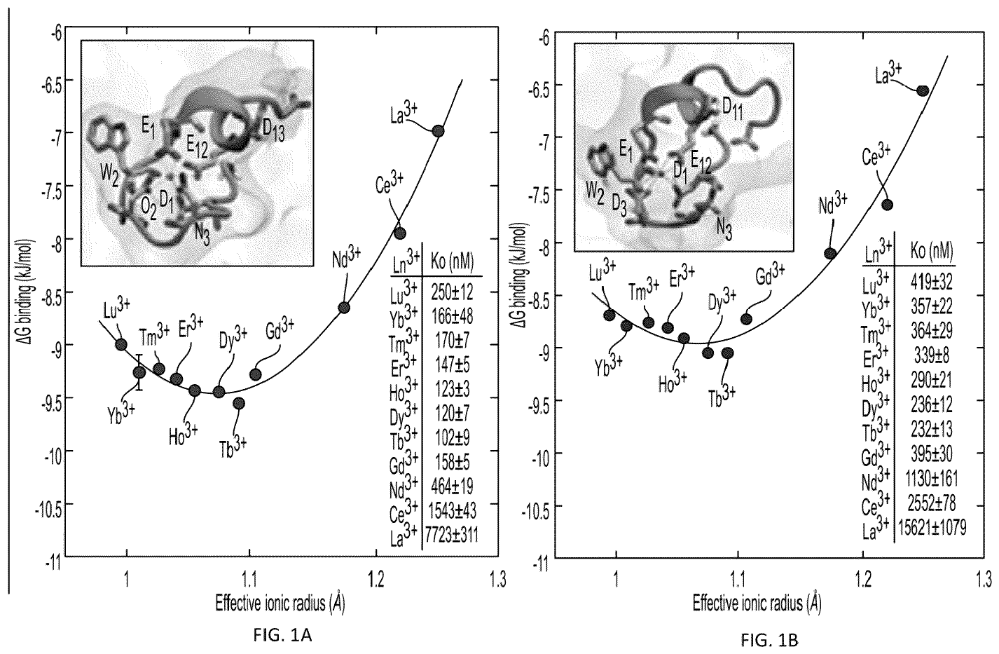

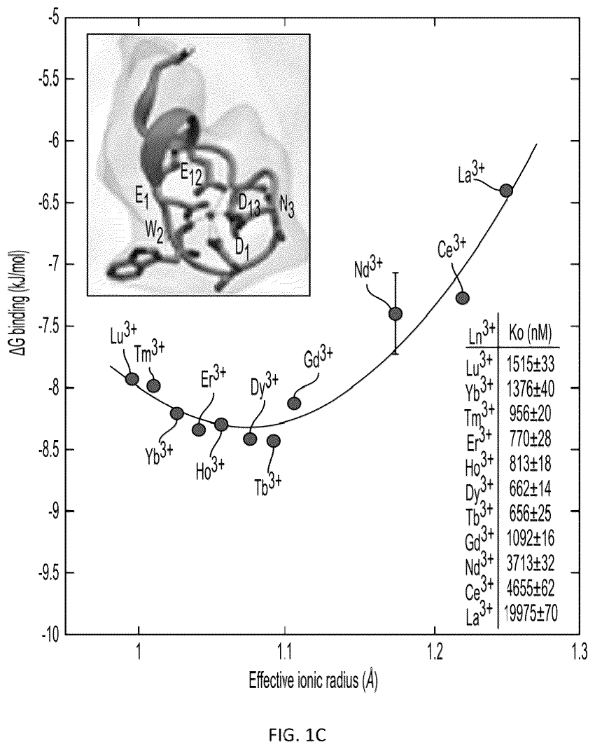

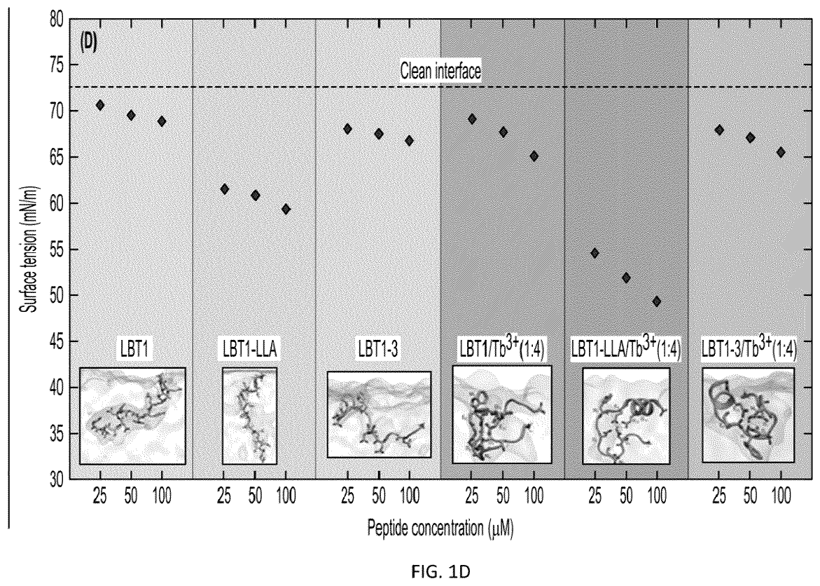

Competitive titration measurements were taken for all peptides LBT1, LBT1-LLA, and LBT1-3 to evaluate the binding affinity of peptides with a series of Ln³⁺ cations. The binding free energy values, shown in FIG. 1A, FIG. 1B, and FIG. 1C show that the reactions are endothermic and spontaneous for all the peptides along the Lanthanides series.

Although measured affinity constants for LBT1 reported in the inset table of FIG. 1A show an approximately two-fold difference compared to values previously reported, the relative affinity for each trivalent cation is similar. The observed differences might be associated with capping of the molecule at the C-terminus or differences in measurement conditions such as pH and temperature. The inset table in FIG. 1B shows that the addition of three hydrophobic residues (-LLA) induced a two-fold increase on the affinity constants, while maintaining the relative affinity for each Ln³⁺ approximately constant. Consistently, the mutations of LBT1 peptide that results in LBT1-3 produce nearly 3-fold increase in affinity dissociation (see inset in FIG. 1C), with similar relative affinity to its homologous LBT peptides. These results show that all peptides in this work can selectively bind with REEs as other LBTs peptides, and the binding affinity depends on the size of each Ln³⁺ ion.

Structures of LBT-Tb³⁺ complexes. Conventional MD simulations are limited in sampling the rugged conformational free energy landscape of the LBTs-Ln³⁺ binding complexes. Well-tempered metadynamics simulations add bias potential to the system total potential along two collective variables to enhance sampling of broad conformational space within reasonable time scale.

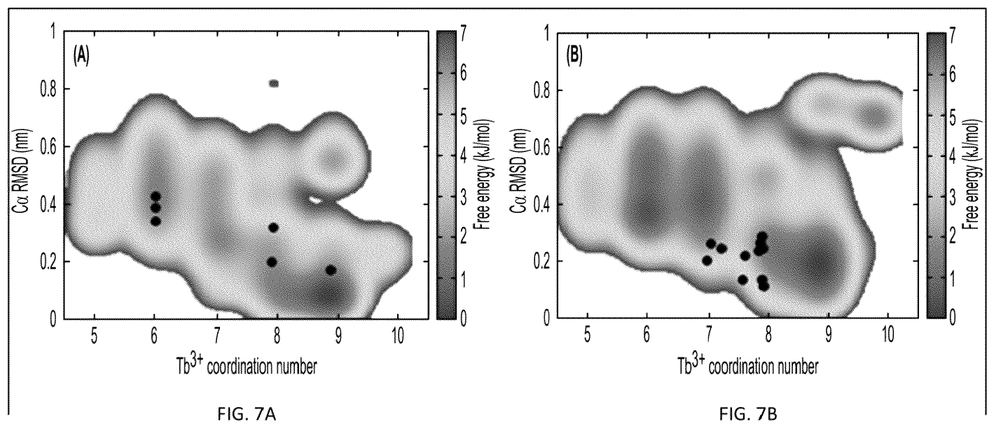

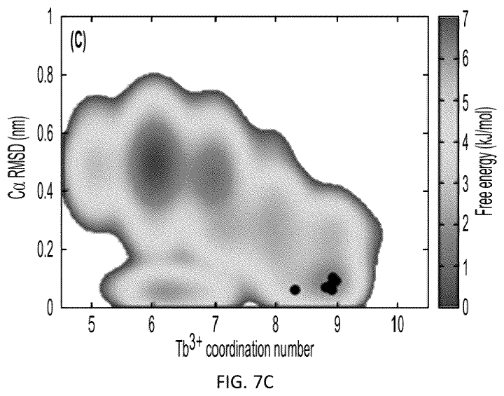

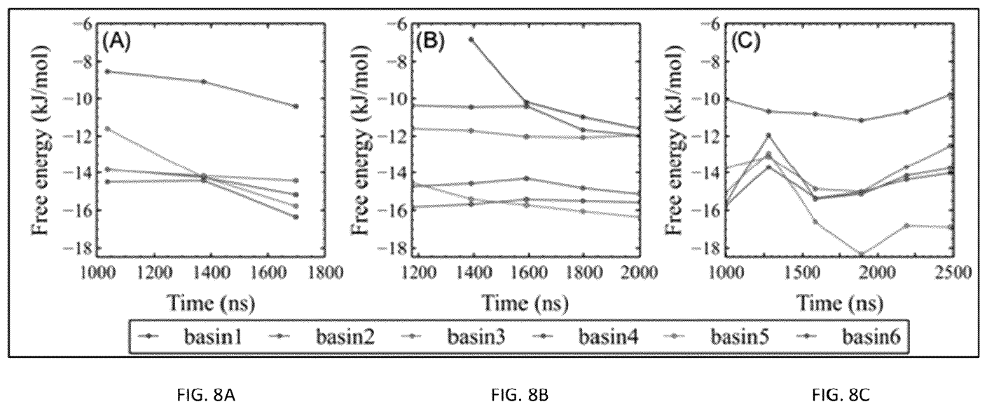

FIG. 7 shows the constructed conformation free energy landscape along the two CVs using histogram reweighting techniques. For each LBT-Tb³⁺ system, there are 5 to 6 distinguishable free energy basins with small free energy differences (ΔΔG≤1 kcal/mol) which correspond to a particular ensemble of binding complex structures, revealing the hidden complexity of the peptide-Tb³⁺ conformations that are hard to capture in experiments. For checking the convergence of the enhanced sampling, free energy values obtained at different simulation times are shown in FIG. 8.

These simulation findings are consistent with the binding energy measurements show in FIGS. 1A-1C, showing that LBT1-3 has a much weaker binding affinity towards Tb³⁺ than LBT1 and LBT1-LLA. As shown in FIG. 7, LBT1 and LBT1-LLA both tend to adopt “tight” binding configurations where 9 oxygen atoms from six residues are in close contact with Tb³⁺. In comparison, LBT1-3 tends to adopt conformations with fewer ligands residues (coordination number ˜6). For the LBT1-LLA/Tb³⁺ complex, even though the more hydrophobic peptide has the same ligating residues as LBT1, the additional LLA motif at the C-terminal increases the degrees of freedom of peptide backbone, allowing for more metastable Tb³⁺ binding conformations with slightly weaker binding affinity than LBT1 peptide.

Final conformations of the microsecond MD simulations for all the LBT/Tb³⁺ complexes are shown as inset snapshoots in FIG. 1A-1C. All structures resulted from microsecond MD simulations are within the conformational free energy landscape and represented as black dots in FIG. 7A, FIG. 7B, and FIG. 7C for LBT1-Tb³⁺, LBT1-LLA/Tb³⁺, and LBT1-3/Tb³⁺ respectively. We see that the peptide molecules wrap around the lanthanide ion forming a pocket through the residues D1, N3, D5, W7, E9, and E12. Details in enumeration of amino acid residue in the peptide chain, counting from the amino terminal are shown in Table S1 for all peptides. The coordination with the ion is through the carboxyl oxygen atoms in D1, D5, E9, and E12 residues, the carbonyl oxygen in N₅, and the backbone oxygen atom of W₇. We notice that the amino acids in the positions from 11 and up tend to form an alpha-helix.

### Surface Tension Measurements

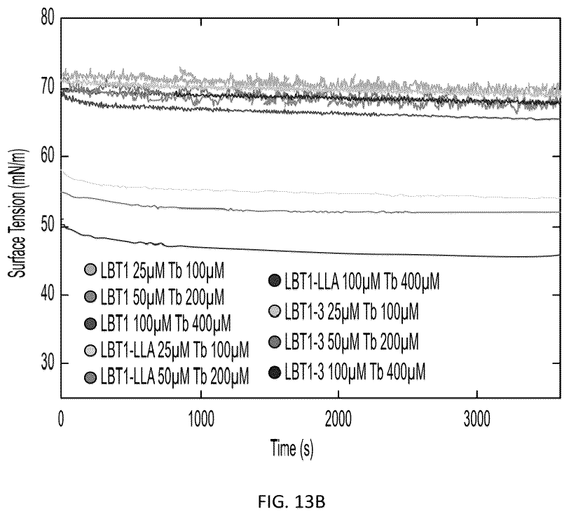

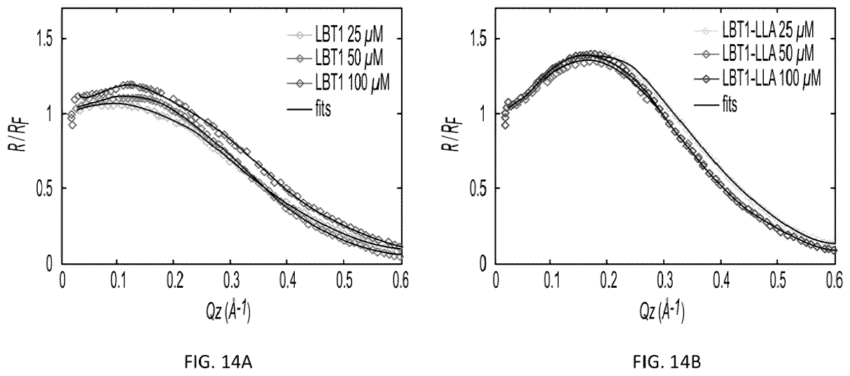

Quasi-equilibrium surface tension measurements of solutions containing LBT1, LBT1-LLA, or LBT1-3 for bulk concentrations of 25 μM, 50 μM, and 100 μM in the absence and presence of Tb³⁺ cations, added in the form of TbCl₃ salt are shown in FIG. 1D. All measurements were performed in the presence of 100 mM of NaCl and 50 mM of MES at a pH of 6.0. The surface tension values reported in this figure correspond to quasi-equilibrium states, obtained after 2 hours (dynamic surface tension values are reposted in FIG. 13). Surface tension values for bound and unbound LBTs from bulk solution to the air-aqueous interface shows a dependence in the bulk concentration; as the peptide concentration in the subphase increases, the surface tension is reduced. This indicates that the higher the bulk concentration of LBTs, the greater the population at the interface and the lower the value of surface tension.

FIG. 1D shows a reduction in the quasi-equilibrium surface tension of LBT1 of values up to around 5% for different bulk concentrations, compared with the surface tension value of a clean interface containing only NaCl and MES. This slight reduction on surface tension validates the surface activity of the Lanthanide Binding Tag peptide LBT1 at the air-aqueous interface, which might be prompted by the hydrophobic residues present towards the N and C terminus of the peptide. The addition of three hydrophobic residues (−LLA) to the peptide LBT1, induces a reduction in the interfacial tension up to around 18% compared to the surface tension of buffer solution. This greater reduction in interfacial tension indicates a higher surface activity of the more hydrophobic peptide compared with the original LBT1, which might represent a greater interfacial coverage due to the presence of more hydrophobic residues in the peptide backbone. The reduction of surface tension obtained for less charged LBT1-3 is up to 8% compared to the value from a clean interface. This greater reduction in surface tension (compared with the original LBT1), might be associated with the absence of greater electrostatic repulsions at the air-aqueous interface that results in a superior surface coverage of LBT molecules.

Surface tension values for the three peptides in the presence of Tb³⁺ cations at different bulk concentration and a ratio peptide:Tb³⁺ of 1:4, are displayed in FIG. 1D. All peptides show a greater reduction in the surface tension values when present in a solution containing Tb³⁺ ions compared with those values from solutions in the absence of the same cations. The more pronounced surface activity observed in the Tb³⁺-bound peptides might be caused by two related phenomena. First, a reduction in the number of negatively charged groups present in the backbone of each peptide diminishes the electrostatic repulsions between individual complexes and allow a more populated interface. Second, the more compact bound molecule occupies a lower molecular surface area (see inset cartoons in FIG. 1D) that results in a more populated interface. The reduction in surface tension from the unbound to the bound state is approximately up to 5%, 17%, and 2% for LBT1, LBT1-LLA, and LBT1-3 respectively. The greater reduction in surface tension observed for LBT1-LLA indicates that even in the bound state the extra hydrophobic residues in the backbone of LBT1-LLA provide a higher surface activity and hence most likely a higher surface coverage of molecules in the interface.

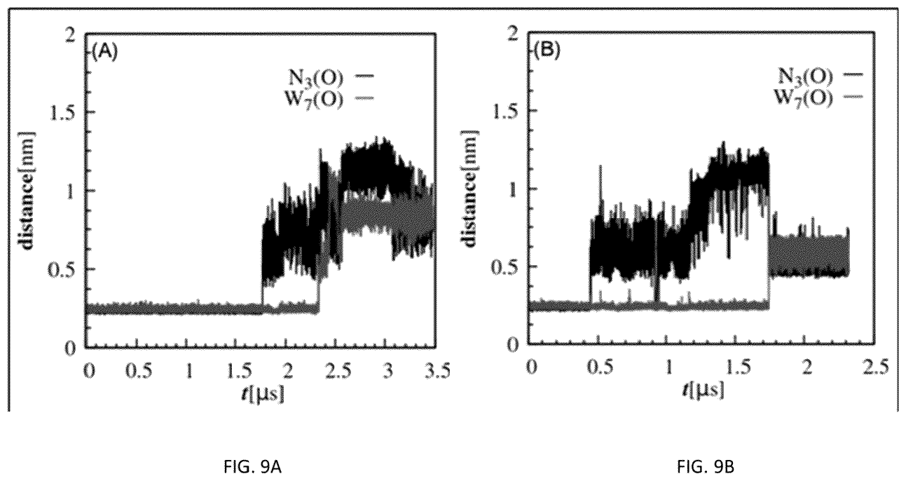

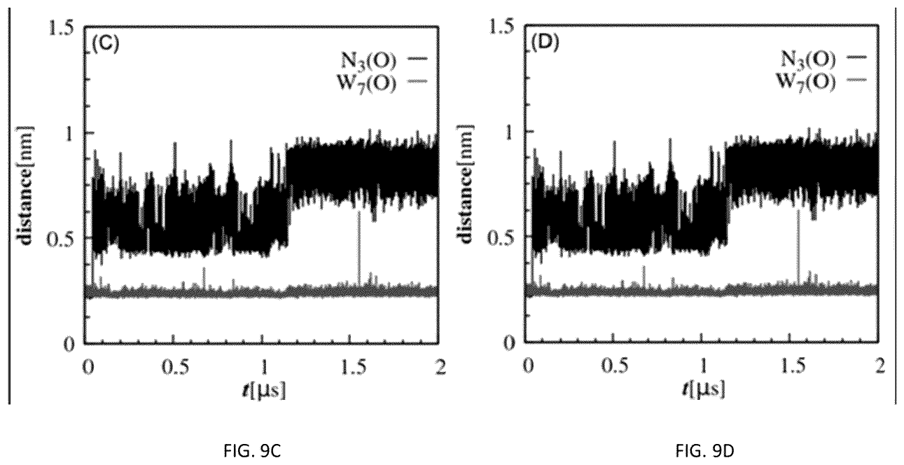

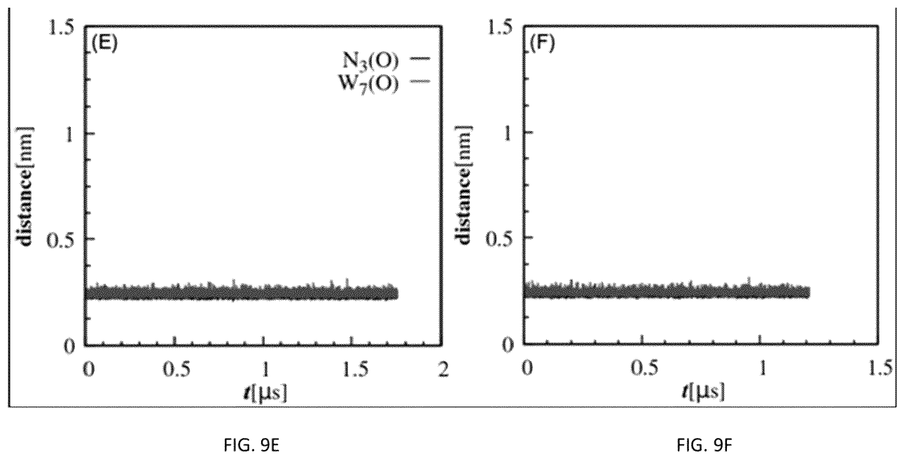

Microsecond MD simulations of complexes at the air-aqueous interface indicate configurational differences between LBTs-Tb³⁺ complexes in bulk and at the interface. In the LBT1 complex in bulk and at the interface, the alpha-helix appears and disappears during the microsecond MD simulations whereas in the LBT1-LLA and LBT1-3 bound peptides the alpha-helix persists. These conformational changes affect the coordination of the trivalent ion in the complex. The coordination distances between the Tb³⁺ ion and the residues are reported in Table S2. In general, either residues D1 or D5 are coordinated only through a single oxygen (monodentate) but the other is coordinated through both oxygens (bidentate). In all the molecules in bulk and at the interface, the residues E9 and E12 are coordinated through both oxygen (bidentate). The coordination with N3 and W7 is unstable. In LBT1 in bulk and at the interface both N3 and W7 detach, and well as in LBT1-LLA at the interface (see FIG. 9). In LBT1-3 the ion coordination with N3 and W7 remains stable above 1 micro-second (shown also in FIG. 9).

### Adsorption of LBT Peptides and Tb³⁺ Cations to the Air-Aqueous Interface

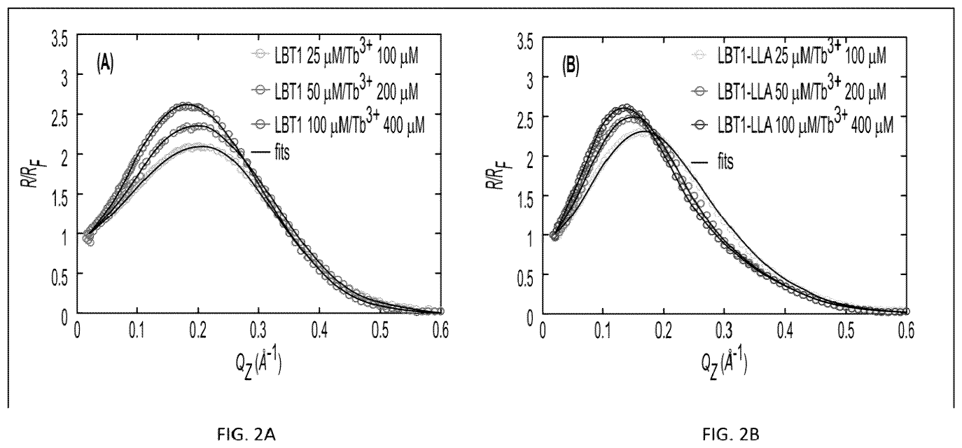

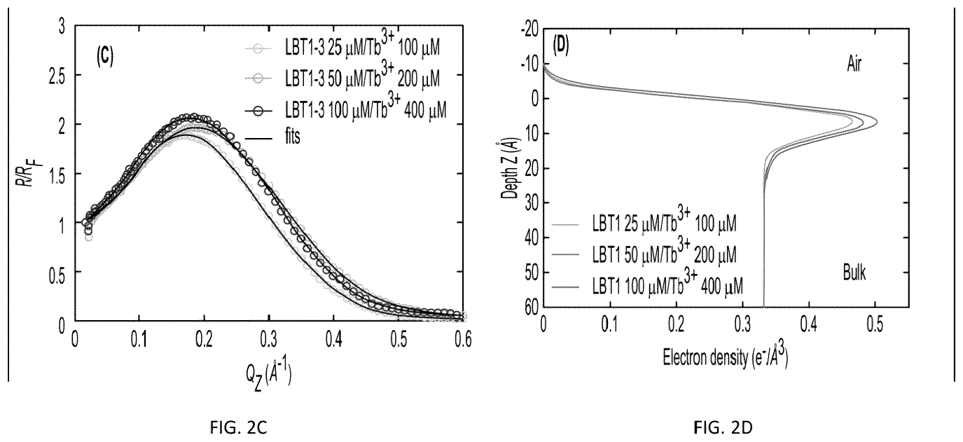

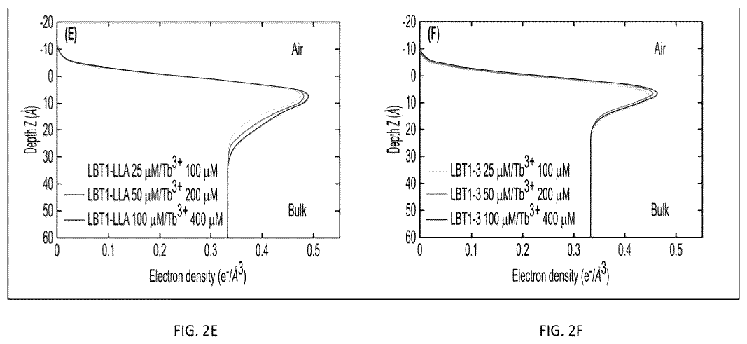

X-ray reflectivity is a technique that uses the x-ray reflection curves resulting from grazing incident x-ray beam to probe the electron density variation ρ(z) normal to the surface z but averaged over the x-y plane. Interfacial molecular quantification is then computed by correlating the measured electron density and the known electron density of the chemical species present at the interface. X-ray reflectivity measurements from the air-aqueous interface and electron density profiles of LBT1/Tb³⁺, LBT1-LLA/Tb³⁺, and LBT1-3/Tb³⁺ adsorbed to an air-aqueous surface are shown in FIG. 2. FIGS. 2A, 2B, and 2C show the normalized reflectivity (R/R_{F}) of individual adsorbed peptides with Tb³⁺ cations as a function of Q_{Z}, and the fitting curve of the XR data obtained using the Parrat method, for which the electron density at the interfacial region is divided into multiple slabs and each slab has its own thickness and electron density. From fitting the x-ray reflectivity data to an electron density profile, we obtained values such as electron density, thickness, and interfacial roughness. These parameters allowed us to compute surface concentration and surface coverage values of species adsorbed to the air-aqueous interface as a function of bulk concentration.

The electron density profile (EDP) of each peptide-cation at different concentrations shown in FIGS. 2D, 2E, and 2F displays the variation of electron density along the air and liquid phases of the interfacial zone with values oscillating between 0 e⁻/Å³ to approximately 0.5 e⁻/Å³. All, LBT/Tb³⁺ samples show an increment in the electron density along z until this parameter reaches a maximum value (higher than the electron density of water) at approximately 10 Å. After this point the electron density drops until it reaches a value of 0.333 e⁻/Å², corresponding to the electron density of bulk water. XR fitted parameters for both peptides were modeled as a single layer of disorder complexes adsorbed to the air-aqueous interface.

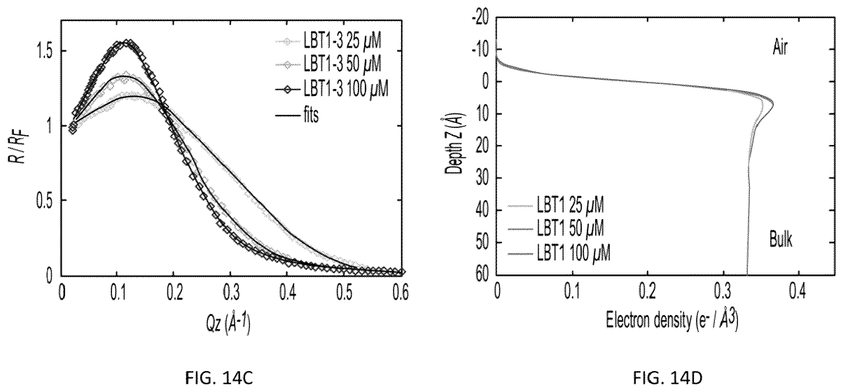

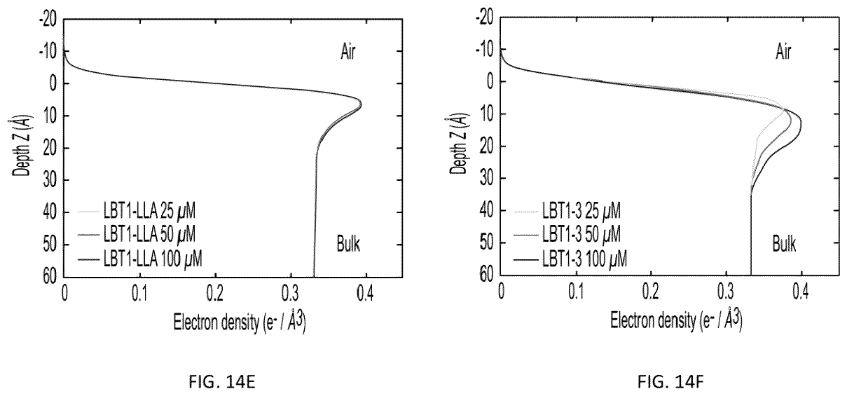

XR measurements and EDPs of adsorbed layers at the air-aqueous interface from solutions containing LBT1, LBT1-LLA, or LBT1-3 are shown in FIG. 14. The normalized reflectivity (R/R_{F}) of adsorbed layers of molecules are shown in FIGS. 14A-14C for each peptide. These reflectivity curves show peaks with lower amplitude at low Q_{Z} compared with the peaks of adsorbed peptides layers when the trivalent cations are in solution. Moreover, the greater reflectivity intensities of adsorbed layers in the presence of Tb³⁺, which indicates a higher electron density at the interfacial zone, are consistent with the surface tension measurements which show that the LBTs/Tb³⁺ complexes are more surface active.

FIGS. 14A-14C show that the amplitude of reflectivity peaks for LBT1-LLA are slightly greater compared to LBT1 and LBT1-3 for the same value of bulk peptide concentration. Moreover, oscillations in the XR curves corresponding to LBT1-LLA/Tb³⁺ layers observed in FIG. 2B are located at a lower Q_{Z} compared to the ones for LBT1/Tb³⁺ and LBT1-3/Tb³⁺ layers (FIGS. 2A, and 2C), which can be associated to a thicker interfacial layer of uniform electron density. EDP of both layers of adsorbed LBT/Tb³⁺ show greater values of electron density along z compared with the ones of free Tb³⁺ peptides. As mentioned above, this might be the result of a higher interfacial density induced by the reduction of electrostatic interactions induced by Tb³⁺ binding and by the more compact conformation of the bound species. Although the maximum value of electron density for layers of all LBT/Tb³⁺ is approximately the same, the distribution of the electron density along the interfacial zone is greater for adsorbed layers of LBT1-LLA/Tb³⁺. Likewise, layers of LBT1-LLA/Tb³⁺ are up to 8 Å thicker compared to LBT1/Tb³⁺ and LBT1-3/Tb³⁺, which might be associated with a higher surface coverage of peptide-cation complexes for LBT1-LLA compared to the less hydrophobic LBT1 and LBT1-3 when they are all in the presence of Tb³⁺ cations.

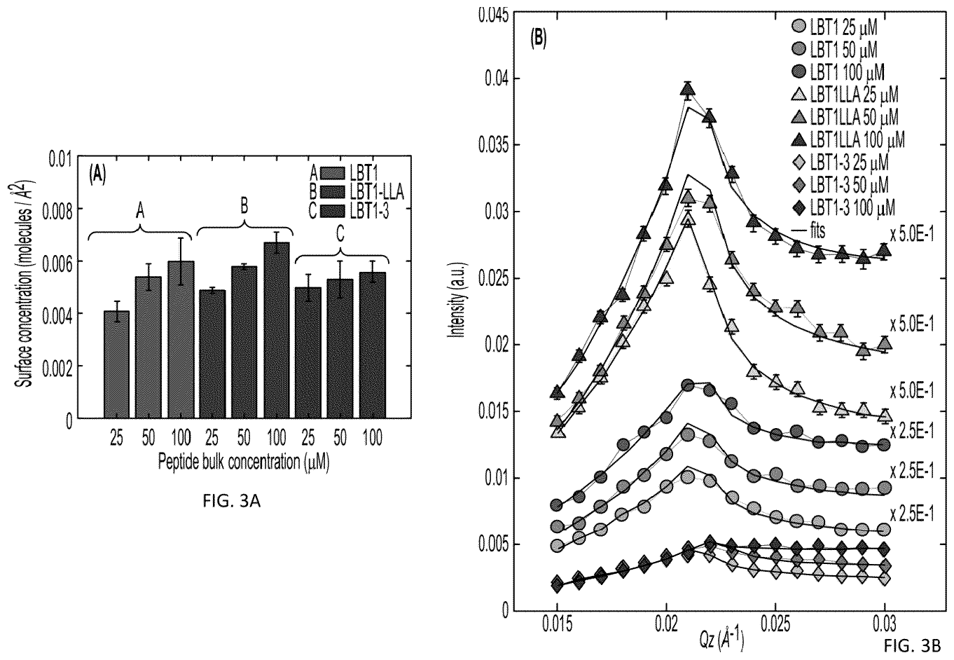

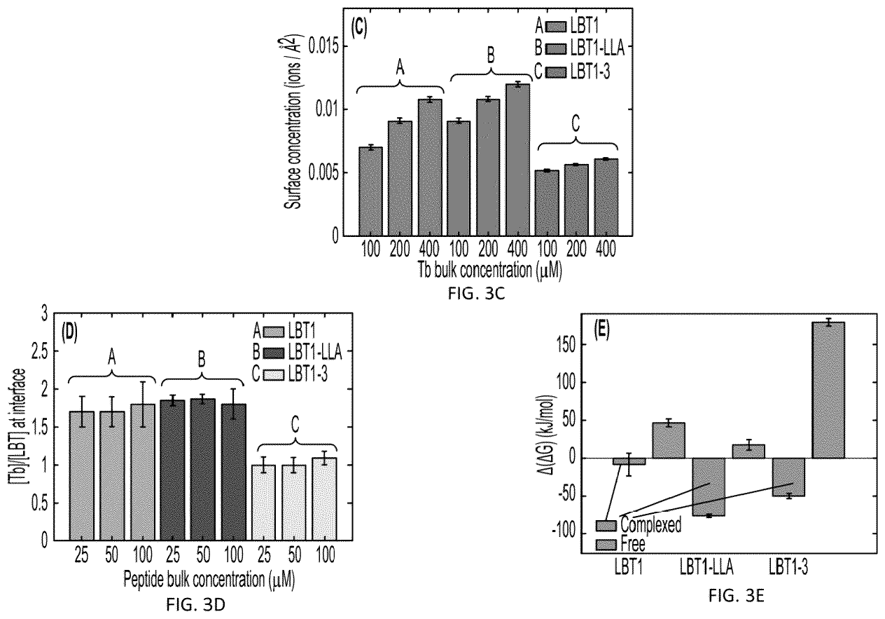

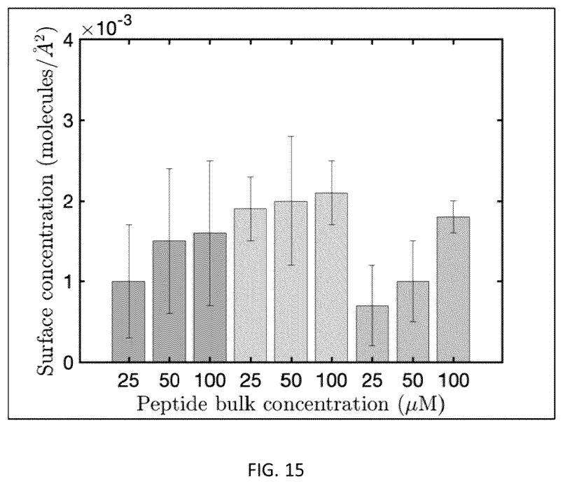

Surface concentration of all peptides obtained from the XR and XFNTR analysis shown in FIG. 3A for the bound peptide and in FIG. 15 for the unbound peptide confirm the surface activity of peptides at the concentrations studied. The adsorption of molecules to the surface might be mainly induced by the hydrophobic residues located at positions 13 to 15 of the backbone of the peptide for LBT1, and LBT1-3 and at positions 13 to 18 for LBT1-LLA. As expected, the addition of the incorporation of -LLA to the backbone of the peptide enhances the surface activity of the molecule, with an increase in surface coverage for LBT1-LLA when Tb³⁺ is not present in solution. The surface activity improvement might be associated with the formation of an extended α-helix, due to the C-terminus of the peptide crystallized with α-helix-like character. Moreover, the surface density of LBT1 and LBT1-3 in the unbound state is nearly equal for the same bulk concentrations. Alternatively, the increase in surface coverage observed for the peptide from the unbound to the bound state is between 3-fold and 4-fold for LBT1, up to three-fold for LBT1-LLA, and approximately up to 5-fold for LBT1-3 for different bulk concentrations of peptide. For the bound species, an increase in peptide surface coverage between 7% and 16% is observed with the addition of -LLA when Tb³⁺ is present in solution, while a reduction in the surface coverage is observed with the reduction of charges of the peptide (LBT1-3).

The recruitment of trivalent cations to the air-aqueous interface is caused by the adsorption of LBTs/Tb³⁺ complexes from bulk solution to the air-aqueous surface. XFNTR was used to determine the interfacial density of trivalent cations. FIG. 3B shows the terbium fluorescence intensity as a function of Q_{Z} near critical angle for total reflection for different concentrations of both LBT, LBT1-LLA, and LBT1-3 for LBTs:Tb³⁺ ratios of 1:4. Terbium fluorescence intensity shows a dependence in the total bulk concentration of peptides and cations; as the bulk concentration of species increases, the fluorescence intensity is higher. This corresponds with both the bulk concentration-dependence of the surface tension values of bound LBTs shown in FIG. 1D, and the bulk concentration-dependence of interfacial density of bound LBTs indicated in FIG. 3A and FIG. 15.

The interfacial density of Tb³⁺ cations as a function of the trivalent salt subphase concentration, illustrated in FIG. 3C, shows an increment in surface concentration between 11% and 30% of Tb³⁺ in the presence of LBT1-LLA compared with LBT1 at constant bulk concentrations of both peptides and cations. Although, the interfacial density of peptide was improved by up to 90% with the introduction of three hydrophobic residues to the backbone of LBT1, a lesser increment of terbium density is observed under the same conditions. This is consistent with the surface tension values presented in FIG. 1D and supports the premise that the binding of Tb³⁺ increases the surface activity of the peptide and enhances its adsorption to the air-aqueous interface. The reduction in the net charge of the LBT1 peptide induced a reduction of up to 44% in the adsorbed Tb³⁺ cations. Moreover, this fall was even greater (up to 49%) when comparing the adsorbed cations with the ones promoted by the more hydrophobic LBT1-LLA peptide.

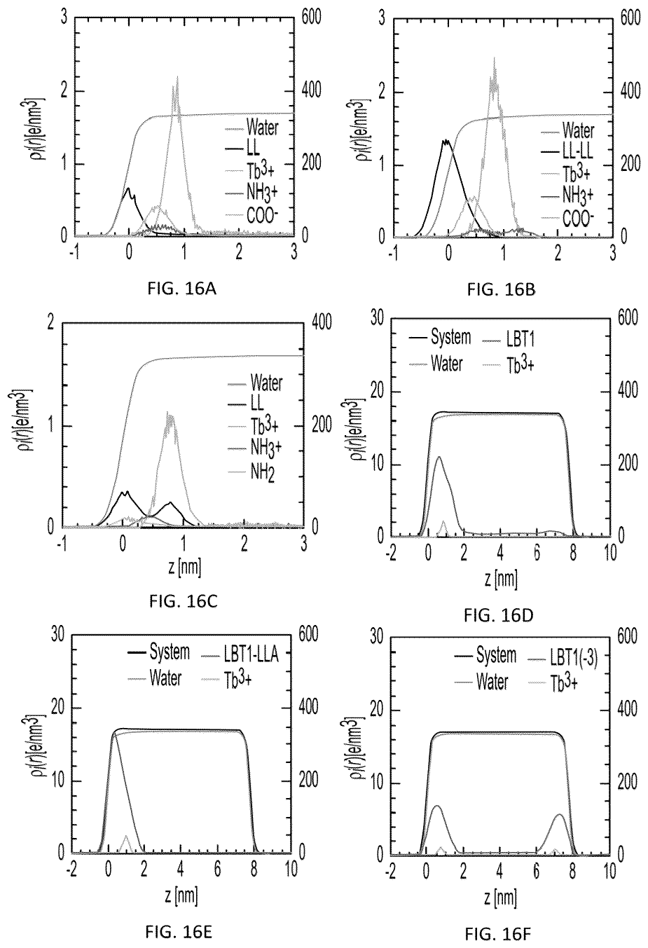

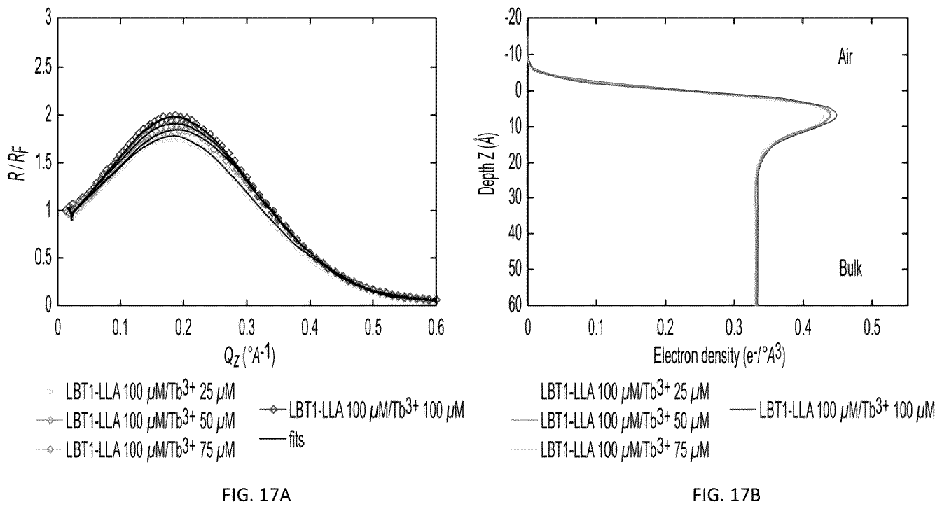

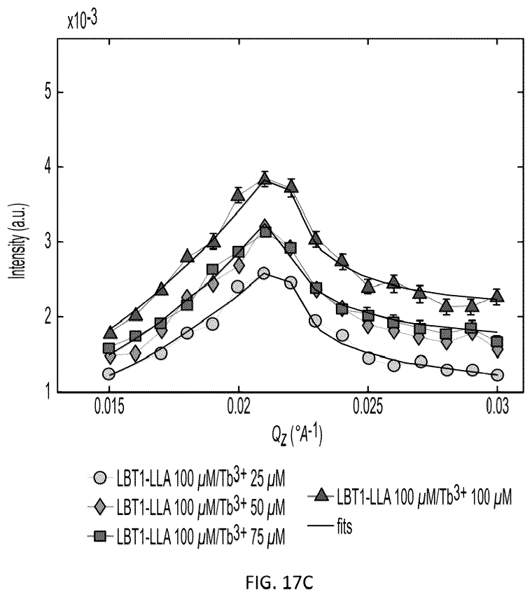

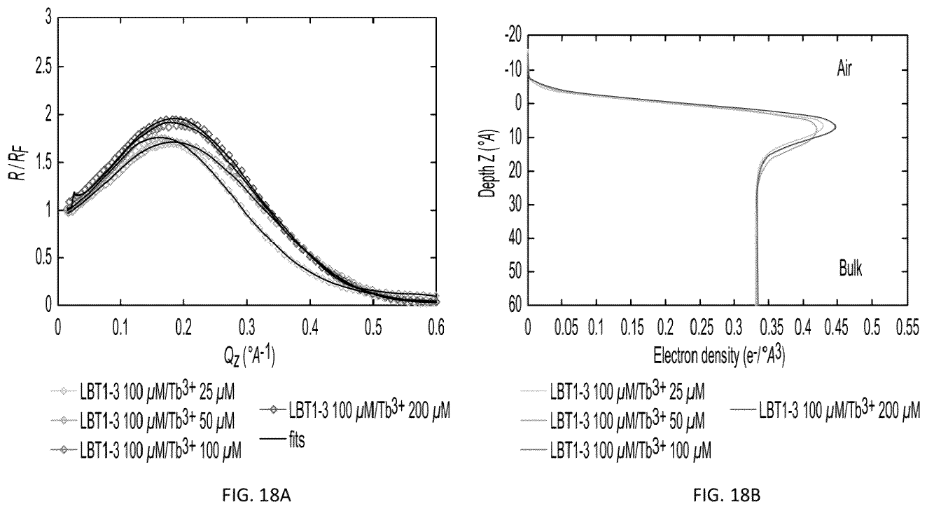

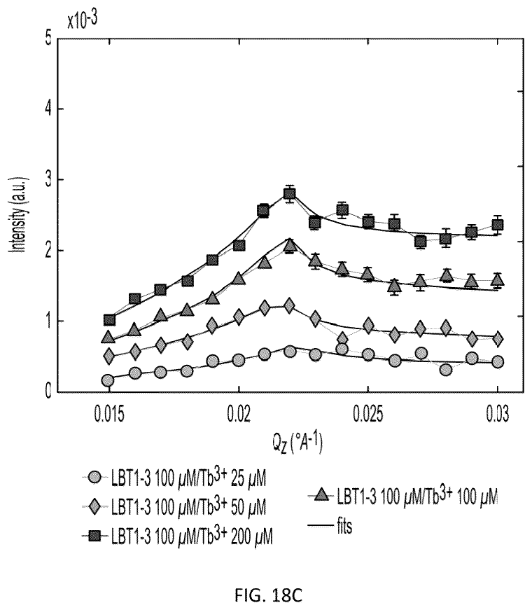

To monitor the location of the hydrophobic section of the molecule with respect to the interface, we investigate the average density profile of different sections of the molecule setting the dividing surface (z=0) at the plane at which the water density is one-half of the bulk density. The most hydrophobic section of the molecules is formed by the amino acids in positions 13 to 15 in LBT1 and LBT1-3, and in positions 13 to 18 in LBT1-LLA (see Table S1). The density profiles in FIGS. 16A, 16B, and 16C show that all three complexes are adsorbed to the interface through the hydrophobic section represented by the position of the outer carbon atoms of the leucine (LL and LL-LL) amino acids. L-carbon atoms are slightly above the dividing surface for LBT1-LLA, at the dividing surface for LBT1, and fluctuating between the dividing surface and at approximately 0.75 nm from the interface for LBT1-3. In all three molecules, the ion is located at about 1 nm from the interface. While the N-terminus is located at about 0.5 nm from the interface for the three complexes, the C-terminus is around 0.5 nm from the interface for LBT1, fluctuates between about 0.5 nm and below 1.2 nm from the interface for LBT1-LLA, and the at the dividing surface for LBT1-3 (see also complexes peptide-Tb³⁺ in inset snapshots in FIG. 1D). Evidently, the changes on the location of the C-terminal of the three complexes once adsorbed to the interface are promoted by the longer hydrophobic but still negatively charged -LLALLA, and the neutral terminal that prefers the hydrophobic section of the interfacial zone.

The change of the free energy (DOG) between the molecules in a bulk solution and at the air-aqueous interface, shown in FIG. 3E, demonstrates that the complex formed by LBT1-LLA is more hydrophobic than the one formed by LBT1 and LBT1-3 (with hydrophobicity as follow LBT1-LLA/Tb³⁺>LBT1-3/Tb³⁺>LBT1/Tb³⁺). Furthermore, the uncomplexed LBT1-LLA has a lower change of free energy compared with the other unbound peptides, with preference towards the interface as follow LBT1-LLA>LBT1>LBT1-3. The higher hydrophobicity of LBT1-LLA is due to the presence of the double LLA sequence at the end of molecule (see Table S1). It is worth noticing that all the molecules are more hydrophobic when they are forming a complex with the lanthanide ion Tb³⁺ than when they are not forming a complex (ion-free), in agreement with the experimental results. Therefore, the molecules are more soluble in water when they are not forming a complex, but they will tend to migrate to the interface when they form a complex with the trivalent ion.

The density profiles of the complexed molecules represented in FIGS. 16D-16F show that once LBT1-LLA/Tb³⁺ is adsorbed, it remains at the air-aqueous interface (this is observed in extended MD runs). Moreover, LBT1/Tb³⁺ stays mostly at the air water interface but frequently it goes away from the interface into the bulk, and LBT1-3/Tb³⁺ has a degree of affinity for the interface and the dissolution in bulk.

### Secondary Binding of Tb³⁺ to LBT Peptides

Quantification of surface concentration of LBT peptides and Tb³⁺ cations allow to determine the number of cations per peptide at the interfacial zone (Γ_{Tb}_{³⁺}/Γ_{LBT}). FIG. 3D shows the ratios of Tb³⁺ to peptide for all three LBT molecules at different bulk concentrations of peptide and with cations at a 1:4 peptide to Tb³⁺ molar ratio (sufficient to saturate the peptides based on the measured affinity constants). Calculated results at the different subphase concentrations show that while for LBT1 and LBT1-LLA the ratio cation to peptide is approximately between 1.7 and 1.87, these values are approximately 1 for LBT1-3.

Although a ratio Γ_{Tb}_{³⁺}/Γ_{LBT} of 1 was expected for the three LBT peptides because structural studies have shown that each LBT coordinate with only one cation, the adsorption of excess Tb³⁺ for the LBT1 and LBT1-LLA molecules is not surprising since LBTs:Ln³⁺ complexes formed by cations in the binding loop are negatively charged (net charge of -2). Thus, the adsorbed layer, constituted by individual peptide-cation complexes, is negatively charged and might trigger a secondary coordination with free trivalent cations in the media. The net charge of both peptides in the apo-state is of -5 according to the charged groups that constitute the backbone of the molecule at a pH of 6, and once the peptide forms the complex with a Ln³⁺ cations the net charge is reduce to a value of −2.

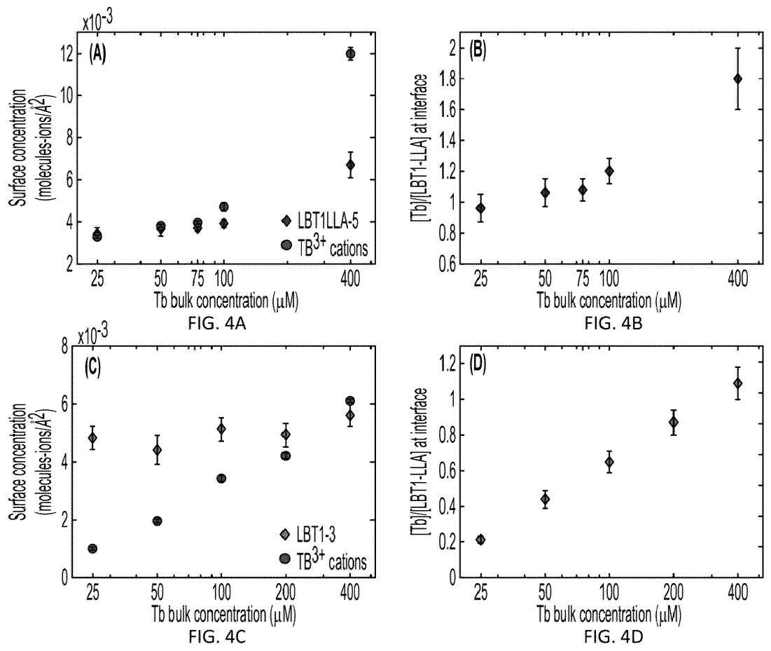

XR and XFNTR measurements of adsorbed layers from solutions containing LBT1-LLA at a constant concentration of 100 μM and Tb³⁺ cations at concentrations varying from 25 μM to 100 μM were taken to determine whether the adsorption of excess cations is induced by the excess unbound cations present in solution. FIG. 4A shows a Tb³⁺ bulk concentration dependence of both peptide and Tb³⁺ interfacial concentration. As the concentration of the trivalent cation in the subphase increases, which represents an increase in either the number of bound species or unbound cations in solution after saturation is reached, the surface coverage of all species increases as well. A 3.5-fold increase in adsorbed concentration of Tb³⁺, and twice the amount of adsorbed peptide is observed for bulk concentration of Tb³⁺ of 400 μM compared with 25 μM. The increment in surface coverage of both peptide and cations as the bulk concentration of Tb³⁺ increases and the bulk concentration of peptide is hold fixed, indicates that the additional free cations in solution promote the adsorption of peptide to the air-aqueous interface. This greater interfacial population of peptide as well as cations, implies that individual complexes might be electrostatically coordinating with each other and forming a network at the interfacial zone. The number of Tb³⁺ cations per peptide adsorbed to the air-aqueous interface in the presence of 100 μM of LBT1-LLA for different concentrations in solution of TbCl₃ are shown in FIG. 4B. For the lowest concentration of Tb³⁺ (25 μM) the ratio is 1.2±0.1, which indicates that although the peptide is not saturated, only bound species are populating the interface and the unbound Tb³⁺ in solution are not sufficient to coordinate with the negatively charged group outside the binding loop. For concentrations of Tb³⁺ of 50 μM and higher we see an increase in the ratio until it reaches the maximum observed value at a concentration of 400 μM of trivalent cations. These results support the hypothesis that free Tb³⁺, in large excess, can associate with the negatively charged groups of the side chain of the peptide that do not participle in the selective binding loop at the interfacial zone.

Without being bound to any particular theory or embodiment, the secondary coordination observed for LBT1 and LBT1-LLA—that promotes the network formation may take place outside of the binding loop of the LBT peptide—may be independent of the ion size, hydration number, or acidity of the metal. Thus, this binding is not discriminatory between different Ln³⁺ cations and hence, not desire in a selective REEs separation process. LBT1-3 peptide, a C-terminal amidated peptide with a total net charge of −3 in the apo-state, does not promote a secondary binding according to the results represented in FIG. 3D. Surface coverage of LBT1 and trivalent cations were determined for undersaturated regimes to study the dependency of adsorbed molecules from the free cations in solution with neutral complexes. FIG. 4C shows the surface concentration of both LBT1-3 molecules and Tb³⁺ cations as a function of cations bulk concentration. The independence of the interfacial coverage of the peptide from the bulk ion concentration, suggests that the amount of free Tb³⁺ in solution does not influence the transport of peptide to the air-aqueous interface, and hence does not promote the adsorption of individual LBT1-3 molecules by bridging individual molecules or complexes. Calculated Γ_{Tb}_{³⁺}/Γ_{LBT1-3}, represented in FIG. 4D, confirms that when the peptide is not saturated with trivalent cations, both bound and unbound peptides are present at the air-aqueous interface. This indicates that LBT1-LLA/Tb³⁺ complexes are more surface active than the uncomplexed LBT1-LLA, while both complexed and uncomplexed LBT1-3 diffuse to the air-aqueous interface similarly at the concentrations studied here.

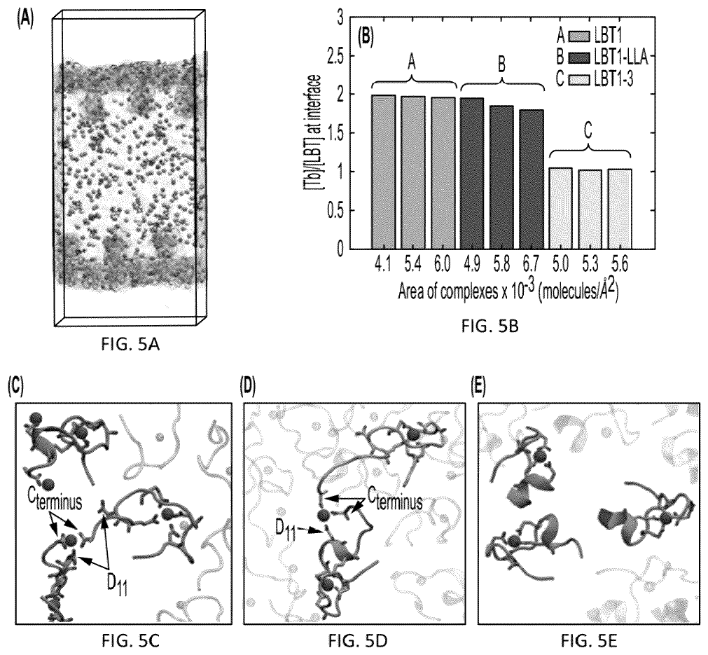

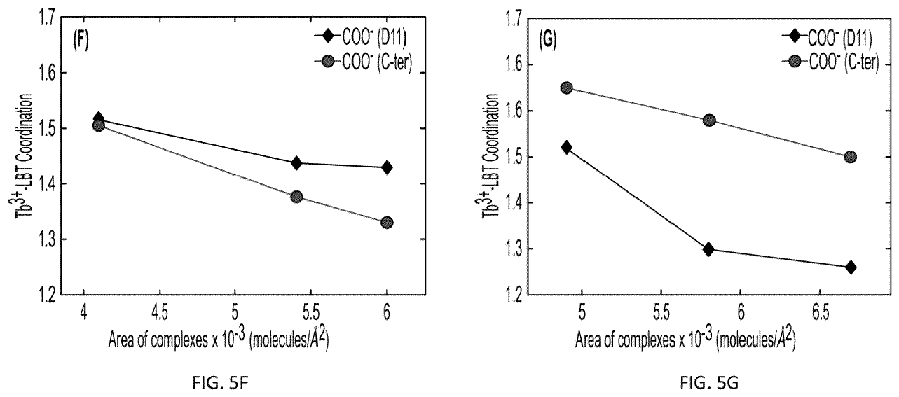

One-microsecond MD simulations are performed to equilibrate the interfacial systems containing the peptide molecules at different surface area concentrations. FIG. 5A shows a system at t 0. The last 400 ns of one-microsecond MD trajectories are used to study the number of adsorbed Tb³⁺ cations per peptide molecules at the interface. A ratio Tb³⁺ to peptide close to 2 at the interface for LBT1 and LBT1-LLA (FIG. 5B) was obtained for all the different molecular areas at the interface (areas obtained from the experimental analysis for the different bulk concentration of peptide and a ratio Tb³⁺/peptide of 4). These results are consistent with experimental results and validate the hypothesis that extra unbound Tb³⁺ in solution can adsorb to the air-aqueous interface for the negatively charged complexes LBT1/Tb³⁺ and LBT1-LLA/Tb³⁺.

FIG. 5C shows a snapshot of a normal view of a highly populated system containing LBT1/Tb³⁺ complexes and Tb³⁺ associating with these complexes. The snapshot shows that the Tb³⁺ cations outside the binding pocket coordinate with the COO⁻ groups from the residue D11 and the C-terminus of the peptide. Moreover, this secondary bridge individual complexes when Tb³⁺ ions associate with COO⁻ groups of different molecules.

FIG. 5B shows that as the molecular interfacial area of the LBT1-LLA cationic complex increases (more populated interface), the number of ion per peptide slightly decreases. This reduction in the number of cations coordinating with the peptide observed only for the more hydrophobic molecule might be associated with the length of the hydrophobic block of the peptide. As shown in FIG. 5D, both COO⁻ outside of the binding loop can coordinate with the same Tb³⁺ ion. Moreover, this already associated O—Tb—O can also coordinate with a third COO⁻ of a different complex. In addition, snapshoots of the populated interfacial layer for both LBT1 and LBT1-LLA show that the secondary ionic coordination is less frequent for LBT1 compared to LBT1-LLA.

Finally, in the secondary coordination for LBT1, the Tb³⁺ ions coordinate preferentially with carboxylate groups (COO—) from the D11 amino acid, whereas in LBT1-LAA, the Tb³⁺ ions have higher coordination with the carboxylate groups from the C-terminus than with the D11 amino acid (FIGS. 5F and FG). The non-integer coordination numbers reflect dynamic coordination and a diverse combination of interconnection possibilities. Oppositely to systems constituted by peptides with a net charge of -5 in the unbound state, in the systems formed by the peptide LBT1-3, the number of Tb³⁺ ions per molecule is about 1 (shown in FIG. 5B). Thus, only the ions that form the complex with the peptide adsorb to the interface. This result is consistent with the experimental results and confirms that Tb³⁺ ions are adsorbed to the interface only via protonated charged groups that conform the selective binding loop as shown in the snapshot of the interfacial film in FIG. 5E.

### Selective Adsorption of REEs to the Air-Aqueous Interface

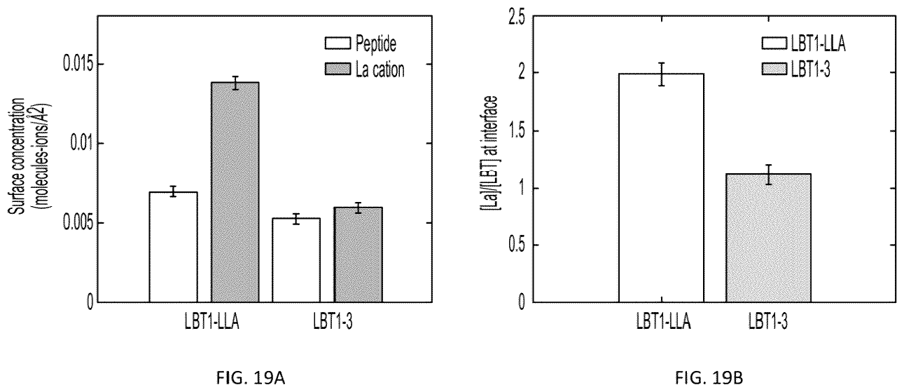

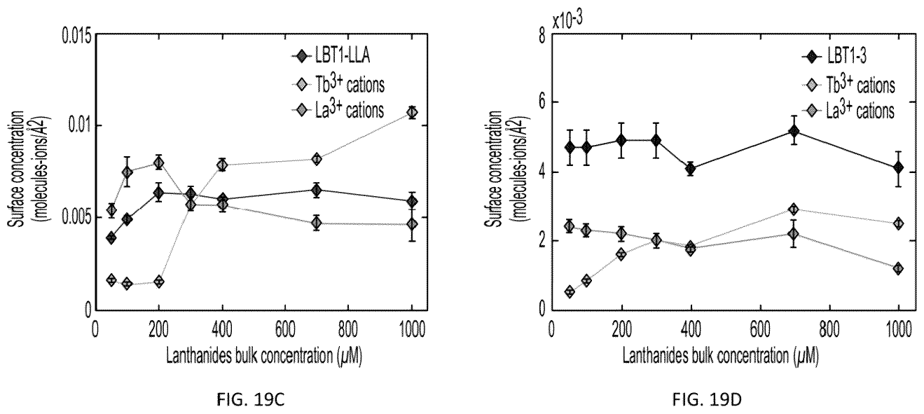

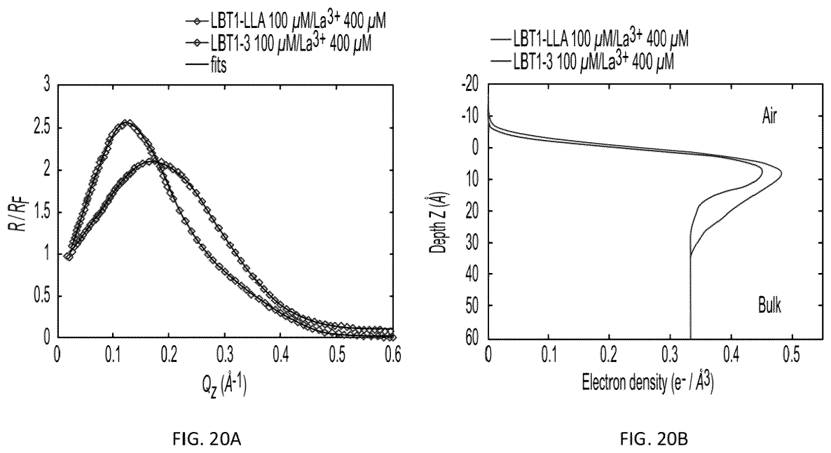

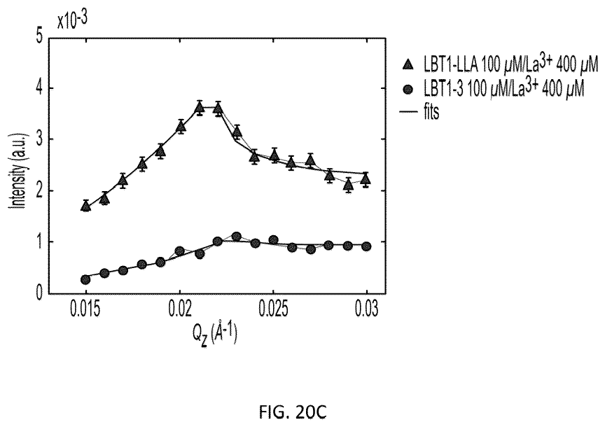

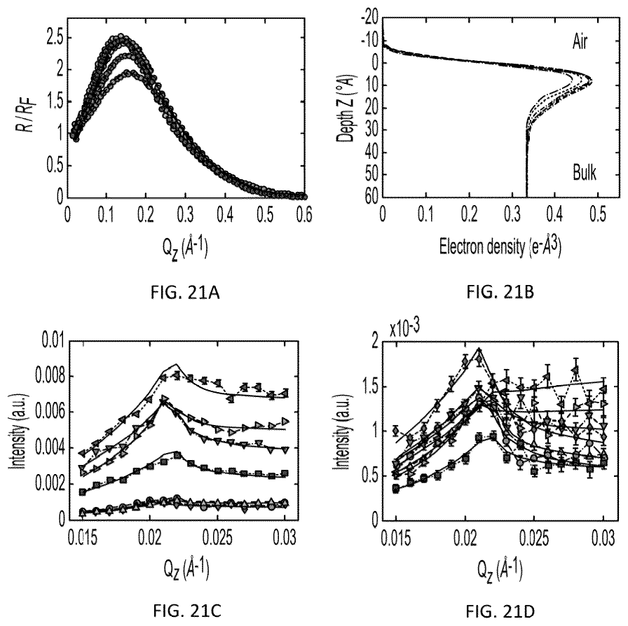

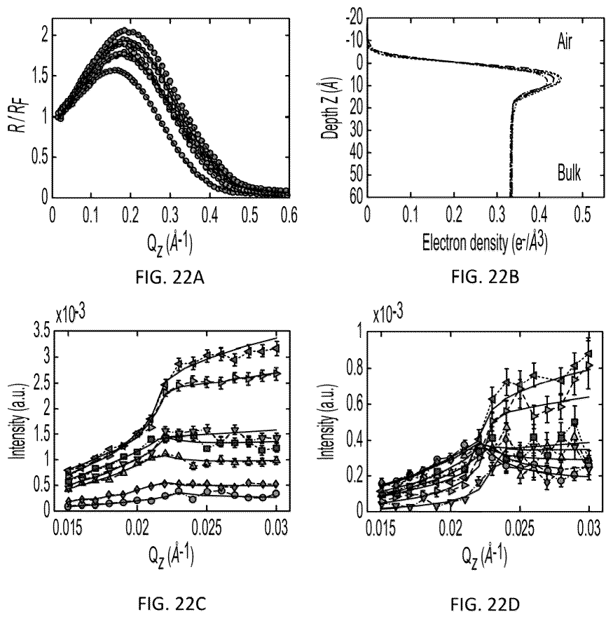

Competitive adsorption between Tb³⁺ and La³⁺ when using LBT peptides as carriers to air-aqueous interfaces was studied. These cations were selected for the competition assays because of the greater difference in affinity with the peptides (see FIGS. 1A-1C). Surface concentration of species was initially calculated for solutions containing either the more hydrophobic and extra charged LBT1-LLA, or the less charged LBT1-3, with La³⁺ cations in solution at a ratio ion to peptide of 4 and at a concentration of peptide of 100 PM. In agreement with the results obtained for Tb³⁺, FIG. 19A shows that while the surface density of both peptides is relatively similar for the same bulk concentration of species, the surface coverage of La³⁺ when LBT1-LLA is in solution approximately doubled the value obtained when using LBT1-3 as an extractant. Furthermore, FIG. 19B shows that the number of La³⁺ per peptide is as well in agreement with the results obtained for Tb³⁺. A secondary coordination takes place when LBT1-LLA/La³⁺ complexes are adsorbed to the interface, that results in excess cations at the interface coordinating with the negatively charged groups outside of the binding loop. As expected for LBT1-3, only one La³⁺ ion per peptide is adsorbed to the interfacial zone.

Surface densities of LBT1-LLA, Tb³⁺, and La³⁺ cations were calculated and are represented in FIG. 19C, for a fixed peptide bulk concentration of 100 μM and different equimolar concentration of lanthanides. As for the single component (only Tb³⁺ cations in solution), a peptide surface concentration dependency on the bulk concentration of trivalent ions is observed for low concentration of lanthanides in solution. As the bulk concentration of total lanthanides increases from 50 μM to 100 μM, and then from 100 μM to 200 μM, the surface density of peptide increases. After this point, the interfacial concentration of peptide is approximately the same for all the bulk concentration of lanthanides (higher and equal to 200 μM). Therefore, the enhancement on adsorption of peptide induced by extra trivalent ions bridging individual LBT1-LLA/Ln³⁺ complexes is not significant for concentration of lanthanides higher than 200 μM, which suggests that at this concentration extra ions in solution are not necessary for the crosslinking of molecules at the interface.

FIG. 19C shows that for bulk concentrations of lanthanides lower than 200 μM the surface coverage of Tb³⁺ remains approximately constant, while the surface coverage of La³⁺ increases as the bulk concentration of Ln increases, in agreement with the increment in surface coverage of peptide. Therefore, the rise in surface density of La ions might be associated with the secondary coordination occurring outside the binding loop that promotes the enhancement of peptide by crosslinking individual LBT1-LLA/Ln³⁺ complexes. The independence of the surface coverage of Tb³⁺ on lanthanides bulk concentration is most likely the result of a lower concentration of these ions in solution in an unbound state, compared to La³⁺ that has a lower affinity with the peptide. For total bulk concentration of lanthanides higher than 200 μM, the surface concentration decreases for La³⁺ and increases for Tb³⁺, as the total Ln bulk concentration increases. This might be associated with the greater affinity of the molecule for Tb³⁺ over La³⁺ observed in the luminescence spectroscopy analysis.

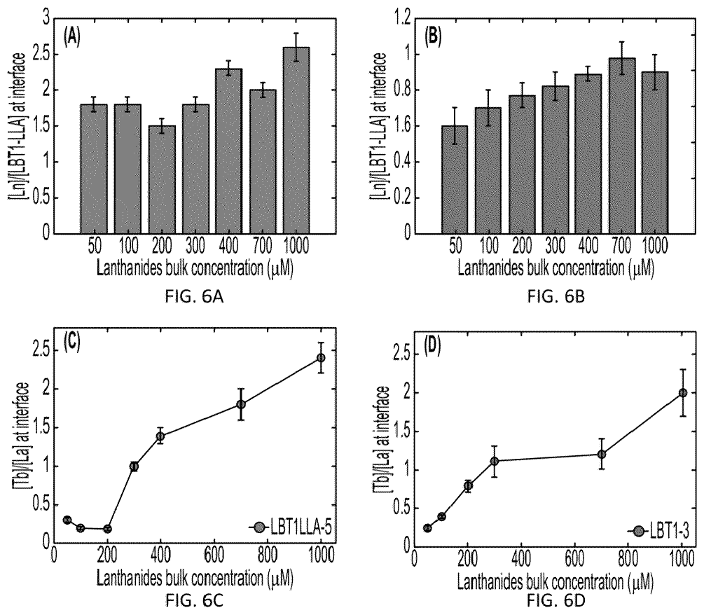

FIG. 6A shows the number of total Ln³⁺ per peptide at the air-aqueous interface calculated from the XR and XFNTR analysis. Oppositely to the interfacial Tb³⁺/LBT1-LLA for undersaturated systems (solutions with a significant amount of unbound peptide) with only the cation in solution with the highest affinity, the number of total Ln³⁺ per LBT1-LLA is higher than 1. This greater interfacial density of Ln³⁺ might be caused by the free La³⁺ cations in solution that do not coordinate with the unbound LBT1-LLA due to its lower affinity with the peptide (higher affinity constant).

Lastly, to evaluate the affinity of LBT1-LLA at the air-aqueous interface, interfacial Tb³⁺/La³⁺ ratios were calculated and are represented in FIG. 6C. While Tb³⁺/La³⁺ values of approximately 1 were expected for undersaturated regimes (Ln bulk concentration of 100 μM and under), values lower than 0.5 were obtained, indicating a greater selectivity for La³⁺ ions at the interface. These unexpected results, as mentioned above, might be the result of free La³⁺ coordinating with the negatively charged groups that do not participate in the selective binding loop. For Ln bulk concentrations higher than 100 μM, Γ_{Tb}_{³⁺}/Γ_{La}_{³⁺} values higher than 1 (with a maximum value of approximately 54 for the highest concentration of total Ln studied) were expected based on the measured association constants. However, interfacial Tb³⁺/La³⁺ ratios lower than 1, equal to 1, and higher than 1 were obtained for bulk concentrations of 200 μM, equal to 400 μM, and higher the 400 μM respectively. Moreover, a maximum Γ_{Tb}_{³⁺}/Γ_{La}_{³⁺} of approximately 2.3 was obtained for the highest concentration of total Ln studied here. In conclusion, the results presented here for LBT1-LLA show changes in the affinity of the molecule for Ln³⁺ cations at the air-aqueous interface. These alterations in the affinity might be caused by a secondary coordination taking place at the interface, which is not considered in the luminescence spectroscopy studies.

Analogously to LBT1-LLA, surface densities of LBT1-3, Tb³⁺, and La³⁺ cations were calculated for a fixed peptide bulk concentration of 100 μM and different equimolar concentration of lanthanides in solution (see FIG. 19D). Opposite to LBT1-LLA and in agreement with the single component studies reported above for LBT1-3, the surface concentration of peptide shows not dependence on the trivalent ions bulk concentration for the different concentration of ions studied here. Moreover, FIG. 6B shows that the total number of ions per peptide at the interface for the different bulk concentration of ions are consistent with the obtained values when only Tb³⁺ cations were in solution (Ln3+/LBT1-3 lower than 1 for low bulk ionic concentrations, and approximately 1 for Ln³⁺ bulk concentrated regimes). This indicates that as for the single components results, when having a mixture of two ions in solution, both unbound and bound peptides adsorbed to the air-aqueous interface.

Surface concentrations of Tb³⁺ and La³⁺ from solutions containing equimolar bulk concentrations of ions and LBT1-3 at a bulk concentration of 100 μM are reported in FIG. 19D. While similarly to LBT1-LLA, the surface concentration of La³⁺ is higher than Tb³⁺ for lanthanides bulk concentration lower than 300 μM, surface concentration increases for Tb³⁺ and decreases for La³⁺. The unexpected results observed here indicate that although the affinity observed in bulk solution is not observed at the air-aqueous interface, the modification of the groups that induce a secondary coordination (substitution of negatively charged group for neutrally charged ones) certainly improve the adsorption of Tb³⁺ cations over La³⁺. This affinity towards Tb³⁺ increases as the concentration of total Ln³⁺ in solution increases, with approximately similar surface coverage of both ions for bulk concentration of Ln³⁺ of 300 μM and 400 μM, and higher affinity towards Tb³⁺ over La³⁺ for Ln³⁺ bulk concentrations higher than 400 μM.

Interfacial Tb³⁺/L³⁺ ratios for different Ln³⁺ bulk concentrations were also calculated and are shown in FIG. 6D. Similarly, to LBT1-LLA values lower than 1 were obtained for undersaturated system. However, it was observed that these values increase as the bulk concentration of total ions increases (not observed for LBT1-LLA for lower concentration of lanthanides in solution). Based on affinity constants measured in solution, interfacial Tb³⁺/L³⁺ values were expected to range from 1 to approximately 23 for the different bulk concentrations studied here. Nonetheless, the greatest affinity for Tb³⁺, which was observed at the highest ionic concentration in solution, showed a Γ_{Tb}_{³⁺}/Γ_{La}_{³⁺} value of approximately 2.

The changes in affinity observed for the neutrally charged-complexed LBT1-3, indicate that changes in affinity of LBT peptides is not only caused by secondary associations occurring outside of the binding loop. The modifications on the affinity of LBT peptides studied here with lanthanides cations might be caused by changes in conformations of complexes once they adsorb to that air-aqueous interfaces (see snapshots in FIGS. 1A-1C for bulk and FIG. 1D for interface). These conformational adjustments, induced by the presence of the hydrophobic interface, result in modifications on atomic Ln-O distances (see Table S2), that are fundamental on the selectivity association with lanthanides cations. Moreover, the conformational changes and the variations on the Ln-O distances for oxygen atoms participating in the binding pocket, must be greater for populated interfaces where intermolecular interactions, such as steric and short-range attractive interaction between individual complexes arise.

These modifications on selectivity seem as well to be dependent on the bulk [Ln]₀/[LBT]₀. For concentration of peptide of 200 μM ([Ln]₀/[LBT]₀=2) and lower, there is a greater affinity for La³⁺ over Tb³⁺.

Moreover, interfacial Ln³⁺/LBT1-3 ratios are lower than 1 (see FIGS. 6B), indicating that there is both uncomplexed and complexed LBT1-3 peptide at the interface; and the interface is not completed saturated with LBT1-LLA peptide complexes which can be observed in the rise of surface concentration of peptide as the bulk Ln concentration increases shown in FIG. 19C). Hence, a lower surface concentration of complexed peptides indicates that the intermolecular interactions at the interface might favor the affinity of both peptides towards La³⁺.

As the lanthanides bulk concentration increases (higher than 200 μM), a higher affinity towards Tb³⁺ is observed, with the surface concentration of the two ions approximately the same for both peptides at [Ln]₀/[LBT]₀ equal to 3, and a greater affinity for Tb³⁺ over La³⁺. While this greater affinity for Tb³⁺ might be related to the higher concentrations of lanthanides in solution, a fully saturated interface is observed for LBT1-LLA, suggesting a rise in molecular interactions between interfacial complexes. Simultaneously, the number of Ln³⁺ per LBT1-3 is higher for this ionic concentration regime, which indicates the presence of mostly complexes at the interfacial zone that results in different kind of interfacial molecular interactions (not negatively charges molecules are present at the interface).

Finally, for Ln³⁺ bulk concentration higher than 300 μM, a higher number of ions per peptide is observed for LBT1-LLA (see FIG. 6A). Although the surface is saturated with LBT1-LLA (shown in FIG. 19C, where a constant surface concentration is observed for Ln³⁺ bulk concentrations higher than 200 μM), the greater number of ions per peptide is most likely related with the smaller ionic radius of Tb over La. Smaller ions might not be able to associate with COO⁻ outside the binding loop of different LBT1-LLA/Ln³⁺ complexes.

### Conclusion

In this discussion, we showed that Lanthanide Binding Tag peptides promote the adsorption of Ln³⁺ cations to air-aqueous interfaces by coordinating with cations in bulk solution and subsequently diffusing to the interface. Moreover, we showed that while the LBT-Ln³⁺ complexes experience conformational changes once they adsorb to the interface, their binding pockets formed in solution are stable at the interfacial zone. Furthermore, the more hydrophobic peptide (LBT1-LLA) can improve the adsorption of Tb³⁺ ions to the air-aqueous interface by up to 30% due to its higher surface activity. This enhanced adsorption is promoted not just by the additional hydrophobic residues of the peptide, but as well by the capacity of the peptide to electrostatically bridge individual complexes with extra free ions in solution. Undesirable association of ions with negatively charged groups from the side chain of the peptide can be eradicated by eliminating the charges participating in this secondary coordination without significantly changing the relative affinity of the peptide for the trivalent cations. LBT peptides studied in this work showed to be selective along the Lanthanide series at air-aqueous interfaces, with affinities different to the ones observed in bulk solution. These affinities indicated to be dependent in the bulk Ln/LBT concentration ratio. Moreover, neutral LBT/Ln³⁺ complexes, such as LBT1-3/Ln³⁺ exhibited a greater selectivity at the air-water interface when comparing it with the bulk one. Finally, the ability to modify the amino acid sequence of LBT peptides and the concentrations of species in solution for controlling the selectivity and extraction of Lanthanide cations to air-aqueous interfaces makes possible their use in green ion foam flotation separation processes of REEs.

Free energy for different basins obtained from well-tempered metadynamics simulations. Convergence of the enhanced sampling in the well-tempered metadynamics simulations was checked by calculating free energy values at different simulations time, reported in FIG. S2Y and calculated using the following equation:

$$〈 {F}_{b a s i n} 〉 = - {k}_{B} T log ( {\int }_{{s}_{1} ( b )}^{{s}_{1} ( a )} {\int }_{{s}_{2} ( d )}^{{s}_{2} ( c )} {e}^{- F ( {s}_{1} , {s}_{2} ) / {k}_{B} T} d {s}_{1} d {s}_{2} ) \qquad ( S 1 )$$

Where [symbol] F_{basin} [symbol] is the free energy value integrated over a given two-dimensional area covered by collective variables s₁ and s₂ which corresponds to the coordination number (CN) of Tb³⁺ and the root mean square deviation of α carbon (Cα-RMSD) of the binding loop domain of LBT peptide respectively. k_{B} is the Boltzmann constant, T is the temperature.

TABLE S1
Amino acid sequence of LBTs peptides using standard nomenclature for residues.

| Peptide | NT | Residues | | | | | | | | | | | | | | | | | | | | CT |
|---|---|---|---|---|---|---|---|---|---|---|---|---|---|---|---|---|---|---|---|---|---|---|
| LBT1 | NH³⁺— | Y | I | D | T | N | N | D | G | W | Y | E | G | D | E | L | L | A | | | | —COO⁻ |
| LBT1-LLA | NH³⁺— | Y | I | D | T | N | N | D | G | W | Y | E | G | D | E | L | L | A | L | L | A | —COO⁻ |
| LBT1-3 | NH³⁺— | Y | I | D | T | N | N | D | G | W | Y | E | G | N | E | L | L | A | | | | —COONH₂ |

| | | | | | | | | | | | | | | | | | | | | |
|---|---|---|---|---|---|---|---|---|---|---|---|---|---|---|---|---|---|---|---|---|
| Position | −1 | 0 | 1 | 2 | 3 | 4 | 5 | 6 | 7 | 8 | 9 | 10 | 11 | 12 | 13 | 14 | 15 | 16 | 17 | 18 |

The first column is the name of the peptide. NT and CT are the N-terminus and C-terminus respectively with charges corresponding to the experimental studies (pH 6). The last row is the position index for each residue. The amino acids coordinating with the ion at t=0 in simulations are colored in blue. The residues that participate in the alpha-helix structure are highlighted using boldface italics.

TABLE S2
Average distances of the binding atoms of the amino acids conforming all the LBT molecules in bulk and at the interface.

| Peptide^{media} | d_{O1D1}[Å] | d_{O2D1}[Å] | d_{O1D5}[Å] | d_{O2D5}[Å] | d_{O1E9}[Å] | d_{O2E9}[Å] | d_{O1E12}[Å] | d_{O2E12}[Å] |
|---|---|---|---|---|---|---|---|---|
| LBT1^{bulk} | 4.12 ± 0.11 | 2.14 ± 0.04 | 2.36 ± 0.31 | 2.57 ± 0.05 | 2.23 ± 0.06 | 2.20 ± 0.05 | 2.78 ± 0.11 | 2.19 ± 0.05 |
| LBT1^{interface} | 4.23 ± 0.07 | 2.15 ± 0.04 | 2.49 ± 0.32 | 2.32 ± 0.32 | 2.25 ± 0.07 | 2.24 ± 0.07 | 2.21 ± 0.06 | 2.30 ± 0.09 |
| LBT1-LLA^{bulk} | 2.14 ± 0.04 | 3.94 ± 0.01 | 2.62 ± 0.34 | 2.18 ± 0.06 | 2.23 ± 0.06 | 2.20 ± 0.06 | 2.34 ± 0.01 | 2.20 ± 0.06 |
| LBT1-LLA^{interface} | 2.22 ± 0.06 | 2.30 ± 0.36 | 4.12 ± 0.14 | 2.62 ± 0.82 | 2.95 ± 0.96 | 2.98 ± 0.96 | 2.20 ± 0.06 | 2.22 ± 0.06 |
| LBT1-3^{bulk} | 3.93 ± 0.11 | 2.14 ± 0.04 | 2.54 ± 0.31 | 2.18 ± 0.06 | 2.22 ± 0.06 | 2.22 ± 0.06 | 2.34 ± 0.11 | 2.22 ± 0.01 |
| LBT1-3^{interface} | 3.93 ± 0.11 | 2.41 ± 0.04 | 2.55 ± 0.31 | 2.17 ± 0.05 | 2.22 ± 0.06 | 2.22 ± 0.05 | 2.34 ± 0.11 | 2.22 ± 0.01 |

d_{O1D1} and d_{O2D1} are the distances from the Tb³⁺ cation to the carboxyl oxygen atoms from the aspartic acid D1; d_{O1D5} and d_{O2D5} are the distances from the Tb³⁺ cation to the carboxyl oxygen atoms from the aspartic acid D5; d_{O1E9} and d_{O2E9} are the distances from the Tb³⁺ cation to the carboxyl oxygen atoms from the glutamic acid E9; and d_{O1E12} and d_{O2E12} are the distances from the Tb³⁺ cation to the carboxyl oxygen atoms from the glutamic acid E12.

TABLE S2
Average distances of the binding atoms of the amino acids conforming all the LBT molecules in bulk and at the interface.

| Peptide^{media} | d_{O1D1}[Å] | d_{O2D1}[Å] | d_{O1D5}[Å] | d_{O2D5}[Å] | d_{O1E9}[Å] | d_{O2E9}[Å] | d_{O1E12}[Å] | d_{O2E12}[Å] |
|---|---|---|---|---|---|---|---|---|
| LBT1^{bulk} | 4.12 ± 0.11 | 2.14 ± 0.04 | 2.36 ± 0.31 | 2.57 ± 0.05 | 2.23 ± 0.06 | 2.20 ± 0.05 | 2.78 ± 0.11 | 2.19 ± 0.05 |
| LBT1^{interface} | 4.23 ± 0.07 | 2.15 ± 0.04 | 2.49 ± 0.32 | 2.32 ± 0.32 | 2.25 ± 0.07 | 2.24 ± 0.07 | 2.21 ± 0.06 | 2.30 ± 0.09 |
| LBT1-LLA^{bulk} | 2.14 ± 0.04 | 3.94 ± 0.01 | 2.62 ± 0.34 | 2.18 ± 0.06 | 2.23 ± 0.06 | 2.20 ± 0.06 | 2.34 ± 0.01 | 2.20 ± 0.06 |
| LBT1-LLA^{interface} | 2.22 ± 0.06 | 2.30 ± 0.36 | 4.12 ± 0.14 | 2.62 ± 0.82 | 2.95 ± 0.96 | 2.98 ± 0.96 | 2.20 ± 0.06 | 2.22 ± 0.06 |
| LBT1-3^{bulk} | 3.93 ± 0.11 | 2.14 ± 0.04 | 2.54 ± 0.31 | 2.18 ± 0.06 | 2.22 ± 0.06 | 2.22 ± 0.06 | 2.34 ± 0.11 | 2.22 ± 0.01 |
| LBT1-3^{interface} | 3.93 ± 0.11 | 2.41 ± 0.04 | 2.55 ± 0.31 | 2.17 ± 0.05 | 2.22 ± 0.06 | 2.22 ± 0.05 | 2.34 ± 0.11 | 2.22 ± 0.01 |

d_{O1D1} and d_{O2D1} are the distances from the Tb³⁺ cation to the carboxyl oxygen atoms from the aspartic acid D1; d_{O1D5} and d_{O2D5} are the distances from the Tb³⁺ cation to the carboxyl oxygen atoms from the aspartic acid D5; d_{O1E9} and d_{O2E9} are the distances from the Tb³⁺ cation to the carboxyl oxygen atoms from the glutamic acid E9; and d_{O1E12} and d_{O2E12} are the distances from the Tb³⁺ cation to the carboxyl oxygen atoms from the glutamic acid E12.

Free energy calculations from MD simulations. The change of the free energy is calculated using the slow-growth method in thermodynamic integration to transform the system from state A to state B as follows:

$${G}^{B} ( T , p ) - {G}^{A} ( T , p ) = {\int }_{0}^{1} {〈 \frac{\partial H}{\partial λ} 〉}_{λ} d λ \qquad ( S2 )$$

l is a coupling parameter that varies from 0 to 1 to modulate the interaction between the target molecule and the medium that is gradually turned in the system's Hamiltonian, such that the interaction between the medium and the molecule is switched off at state A (l=0) and is on in state B (l=0).

$$Δ G = {\int }_{0}^{1} {〈 \frac{\partial H}{\partial λ} 〉}_{λ} d λ \qquad ( S3 )$$

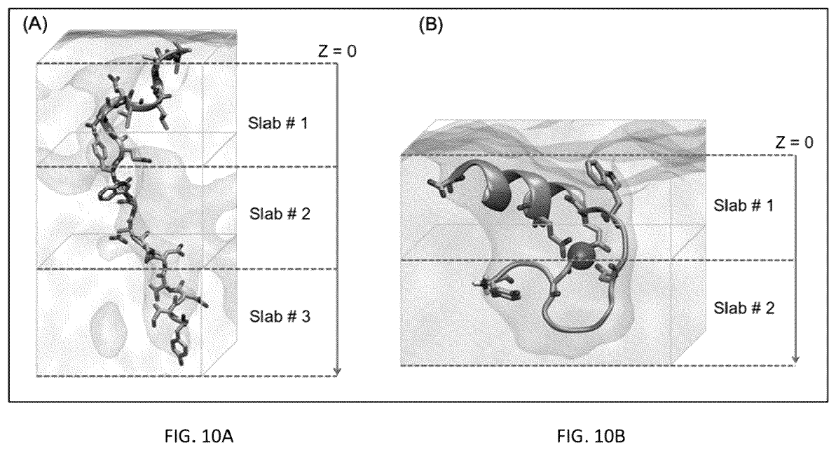

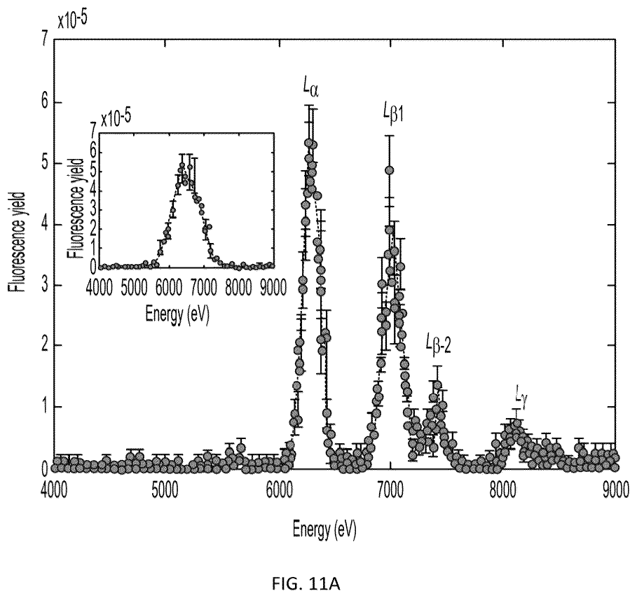

X-ray Reflectivity (XR) Data Analysis. Reflectivity data was normalized by the Fresnel Reflectivity (R_{F}), which is the result of x-rays reflecting from an ideal interface. (R/R_{F}) as a function of the wave vector transfer perpendicular to the interface (Q_{Z}) is fit to determine the electron density ρ(z) along the direction perpendicular to the surface. Two- and three-slab models were used to fit the reflectivity data to the model functional, as represented in FIG. 10. For all the fittings, a minimum number of two slabs were used. Water molecules are also part of the molecular species present in the polar slabs of the interface.

### Surface Concentration of Molecules at Interface

From the corresponding fitting of the XR data, we obtained values such as electron density, thickness, and interfacial roughness (Tables S4-S5). These parameters allowed us to compute surface concentration of peptide adsorbed to the air-water interface for each LBTs and LBTs/Tb³⁺ adsorbed layers to the air-aqueous interface from bulk solution. To compute the surface concentrations of molecules populating the air water interface we applied electron density and molar volume balances resulting in:

$${e}_{T} = {Γ}_{L B T s} {e}_{L B T s} + {Γ}_{T {b}^{3 +}} {e}_{T {b}^{3 +}} + {Γ}_{w} {e}_{w} \qquad ( S3 )$$

$${d}_{T} = {Γ}_{L B T s} {V}_{L B T s} + {Γ}_{T {b}^{3 +}} {V}_{T {b}^{3 +}} + {Γ}_{w} {V}_{w} \qquad ( S4 )$$

with,
   d_{T} (Å) as the total thickness of the layer:

$${d}_{T} = \sum {d}_{i} = {d}_{1} + {d}_{2} \qquad ( S5 )$$

e_{T} (electrons/Å²) as the electron density of the air-water interface:

$${e}_{T} = \sum {d}_{i} {ρ}_{i} = {d}_{1} {ρ}_{1} + {d}_{2} {ρ}_{2} \qquad ( S6 )$$

e_{w} as the number of electrons in each water molecule (10 e/molecule).
V_{W} as the molar volume of water molecules (30 Å³/molecule).
e_{Tb}_{³⁺} as the number of electrons in Terbium cation (62 electrons/ion).
V_{Tb}_{³⁺} is the partial molar volume of Tb³⁺ ions, calculated from the ionic radius in standard tables and with a value of approximately 5.43 Å³/ion. [4]
e_{LBTs} as the number of electrons in each LBT peptide cation (1054 electrons/molecule for LBT1 and 126 electrons/molecule for LBT1-LLA).
V_{LBTs} is the partial molar volume of each LBT peptide calculated from MD simulations.
Γ_{LBTs} and Γ_{W} are the surface concentration (molecule/Å²) of LBT peptide and water respectively (both unknown).
Γ_{Tb}_{³⁺} is the surface concentration Tb³⁺ ions obtained from the x-ray fluorescence near total reflection (XFNTR) analysis (see below).

### X-Ray Fluorescence Near Total Reflection (XFNTR)

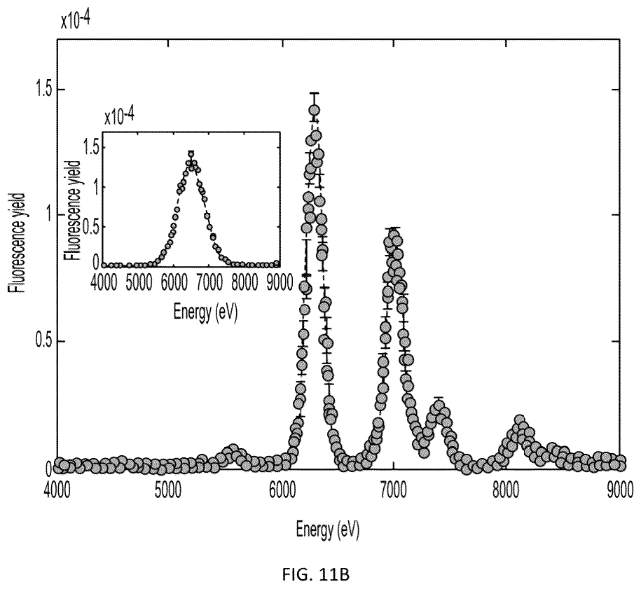

XFNTR is used to calculate the coverage of Tb³⁺ cations at the air-aqueous interface. FIG. 11A shows the L_{α}, L_{β1}, L_{β2}, and L_{γ} fluorescence peaks of Tb³⁺ from a solution containing 25 mM of TbCl₃ (reference sample). The same peaks can be distinguished in the fluorescence spectrum of the solutions containing Tb³⁺ cations and peptide shown in FIG. 11B. L_{α} fluorescence peak of terbium was fit to a Gaussian function over the energy range from 5900 eV to 6700 eV—shown in the inset plot of FIG. 11A and FIG. 11B- to obtain its integrated area, which provides the XFNTR signal.

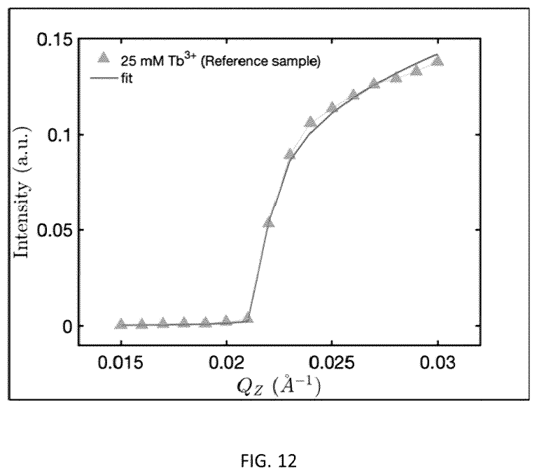

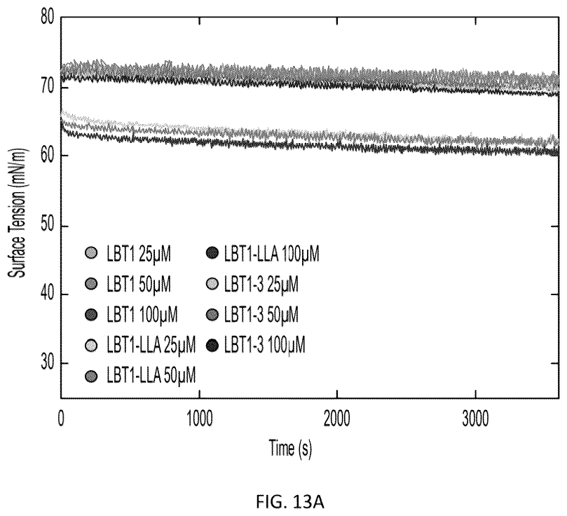

The integrated fluorescence intensity of Tb³⁺ L_{α} emission lines measured for values of Q_{Z} near the critical value (Q_{c}) for the reference sample is represented in FIG. 12. Its overall shape varies to the measurement from the studied samples (solutions containing Tb³⁺ and peptides shown in FIG. 3B) at high Q_{Z}, implying that the bulk contribution dominates the signal. The fluorescence integration, and further XFNTR fitting for the reference sample was done in order to obtain a scale factor determined by the scattering geometry and elements, which is necessary to obtain the interfacial coverage of Tb³⁺ cations at the air-aqueous interface [2].

TABLE S3
Fitting parameters from XR measurements from adsorbed layers of solutions
containing LBT1, LBT1-LLA, or LBT1-3 at different bulk concentrations.

| Slab | LBT1 25 μM | | | LBT1 50 μM | | | LBT1 100 μM | | |
|---|---|---|---|---|---|---|---|---|---|
| # | d (Å) | ρ (el./Å³) | σ (Å) | d (Å) | ρ (el./Å³) | σ (Å) | d (Å) | ρ (el./Å³) | σ (Å) |
| 1 | 11.8^{±0.1} | 0.3551^{±0.0003} | 2.575^{±0.005} | 10.8^{±0.3} | 0.370^{±0.001} | 2.82^{±0.02} | 10.6^{±0.4} | 0.371^{±0.002} | 2.49^{±0.02} |
| 2 | 10.9^{±0.5} | 0.3373^{±0.0002} | 2.575^{±0.005} | 9.4^{±0.5} | 0.3391^{±0.0004} | 2.82^{±0.02} | 9.1^{±0.3} | 0.343^{±0.001} | 2.49^{±0.02} |
| 3 | 14.9^{±0.6} | 0.3349^{±0.0002} | 2.575^{±0.005} | 12.0^{±0.4} | 0.3345^{±0.0001} | 2.82^{±0.02} | 10.4^{±0.6} | 0.3360^{±0.0005} | 2.49^{±0.02} |
| Slab | LBT1-LLA 25 μM | | | LBT1-LLA 50 μM | | | LBT1-LLA 100 μM | | |
| # | d (Å) | ρ (el./Å³) | σ (Å) | d (Å) | ρ (el./Å³) | σ (Å) | d (Å) | ρ (el./Å³) | σ (Å) |
| 1 | 10.8^{±0.1} | 0.3985^{±0.0007} | 2.42^{±0.01} | 9.9^{±0.4} | 0.404^{±0.008} | 2.69^{±0.09} | 10.8^{±0.2} | 0.402^{±0.001} | 2.65^{±0.02} |
| 2 | 8.4^{±0.1} | 0.3438^{±0.0004} | 2.42^{±0.01} | 7.3^{±0.2} | 0.349^{±0.003} | 2.69^{±0.09} | 7.8^{±0.1} | 0.3476^{±0.0009} | 2.65^{±0.02} |
| 3 | 12.9^{±0.4} | 0.3348^{±0.0001} | 2.42^{±0.01} | 11.9^{±0.7} | 0.3356^{±0.0004} | 2.69^{±0.09} | 12.7^{±0.3} | 0.3352^{±0.0001} | 2.65^{±0.02} |
| Slab | LBT1-3 25 μM | | | LBT1-3 50 μM | | | LBT1-3 100 μM | | |
| # | d (Å) | ρ (el./Å³) | σ (Å) | d (Å) | ρ (el./Å³) | σ (Å) | d (Å) | ρ (el./Å³) | σ (Å) |
| 1 | 2.4^{±0.4} | 0.20^{±0.04} | 2.9^{±0.1} | 4.7^{±0.2} | 0.25^{±0.01} | 2.96^{±0.07} | 5.62^{±0.04} | 0.263^{±0.002} | 2.92^{±0.02} |
| 2 | 10.7^{±0.3} | 0.384^{±0.002} | 2.9^{±0.1} | 13.5^{±0.4} | 0.3862^{±0.0007} | 2.96^{±0.07} | 14.8^{±0.1} | 0.4014^{±0.0004} | 2.92^{±0.02} |
| 3 | 13.0^{±0.2} | 0.341^{±0.0003} | 2.9^{±0.1} | 9.8^{±0.2} | 0.3445^{±0.0005} | 2.96^{±0.07} | 9.33^{±0.08} | 0.3511^{±0.0003} | 2.92^{±0.02} |

TABLE S4
Fitting parameters from XR measurements from adsorbed layers of solutions containing
LBT1/Tb³⁺, LBT1-LLA/Tb³⁺, or LBT1-3/Tb³⁺ at different bulk concentrations.

| Slab | LBT1 25 M/Tb³⁺ 100 M | | | LBT1 50 μM/Tb³⁺ 200 μM | | | LBT1 100 μM/Tb³⁺ 400 μM | | |
|---|---|---|---|---|---|---|---|---|---|
| # | d (Å) | ρ (el./Å³) | σ (Å) | d (Å) | ρ (el./Å³) | σ (Å) | d (Å) | ρ (el./Å³) | σ (Å) |
| 1 | 10.07^{±0.07} | 0.504^{±0.002} | 3.34^{±0.02} | 10.3^{±0.1} | 0.545^{±0.005} | 3.44^{±0.03} | 10.0^{±0.3} | 0.58^{±0.01} | 3.81^{±0.09} |
| 2 | 15.9^{±0.3} | 0.3337^{±0.0002} | 3.34^{±0.02} | 14.3^{±0.3} | 0.340^{±0.001} | 3.44^{±0.03} | 13.4^{±093} | 0.3389^{±0.0007} | 3.81^{±0.09} |
| Slab | LBT1-LLA 25 μM/Tb³⁺ 100 μM | | | LBT1-LLA 50 μM/Tb³⁺ 200 μM | | | LBT1-LLA 100 μM/Tb³⁺ 400 μM | | |
| # | d (Å) | ρ (el./Å³) | σ (Å) | d (Å) | ρ (el./Å³) | σ (Å) | d (Å) | ρ (el./Å³) | σ (Å) |
| 1 | 11.81^{±0.06} | 0.4909^{±0.0009} | 3.26^{±0.01} | 12.59^{±0.06} | 0.4953^{±0.0007} | 3.45^{±0.01} | 11.8^{±0.2} | 0.513^{±0.002} | 3.63^{±0.03} |
| 2 | 8.53^{±0.04} | 0.3669^{±0.0005} | 3.26^{±0.01} | 9.02^{±0.03} | 0.3868^{±0.0006} | 3.45^{±0.01} | 9.70^{±0.07} | 0.407^{±0.001} | 3.63^{±0.03} |
| 3 | — | — | — | — | — | — | 8.2^{±0.3} | 0.3421^{±0.0007} | 3.63^{±0.03} |
| Slab | LBT1-3 25 μM/Tb³⁺ 100 μM | | | LBT1-3 50 μM/Tb³⁺ 200 μM | | | LBT1-3 100 μM/Tb³⁺ 400 μM | | |
| # | d (Å) | ρ (el./Å³) | σ (Å) | d (Å) | ρ (el./Å³) | σ (Å) | d (Å) | ρ (el./Å³) | σ (Å) |
| 1 | 11.72^{±0.03} | 0.4687^{±0.0008} | 3.51^{±0.01} | 8.7^{±0.2} | 0.524^{±0.007} | 3.59^{±0.04} | 9.2^{±0.2} | 0.512^{±0.003} | 3.55^{±0.02} |
| 2 | 9.3^{±0.3} | 0.3386^{±0.0003} | 3.51^{±0.01} | 6.5^{±0.3} | 0.346^{±0.002} | 3.59^{±0.04} | 4.87^{±0.09} | 0.376^{±0.004} | 3.55^{±0.02} |

TABLE S5
Fitting parameters from XR measurements from adsorbed layers of solutions containing
100 μM of LBT1-LLA and different concentrations of Tb³⁺.

| Slab | Tb³⁺ 25 μM | | | Tb³⁺ 50 μM | | |
|---|---|---|---|---|---|---|
| # | d (Å) | ρ (el./Å³) | σ (Å) | d (Å) | ρ (el./Å³) | σ (Å) |
| 1 | 11.31^{±0.09} | 0.4418^{±0.0009} | 2.93^{±0.01} | 11.16^{±0.08} | 0.451^{±0.001} | 2.91^{±0.01} |
| 2 | 7.7^{±0.2} | 0.3446^{±0.0006} | 2.93^{±0.01} | 7.6^{±0.2} | 0.3437^{±0.0006} | 2.91^{±0.01} |
| Slab | Tb³⁺ 75 μM | | | Tb³⁺ 100 μM | | |
| # | d (Å) | ρ (el./Å³) | σ (Å) | d (Å) | ρ (el./Å³) | σ (Å) |
| 1 | 11.42^{±0.05} | 0.4517^{±0.0007} | 2.88^{±0.01} | 11.33^{±0.05} | 0.4625^{±0.0008} | 2.97^{±0.01} |
| 2 | 8.1^{±0.2} | 0.3436^{±0.0004} | 2.88^{±0.01} | 8.7^{±0.1} | 0.3446^{±0.0003} | 2.97^{±0.01} |

TABLE S6
Fitting parameters from XR measurements from adsorbed layers of solutions
containing 100 μM of LBT1-3 and different concentrations of Tb³⁺.

| Slab | Tb³⁺ 25 μM | | | Tb³⁺ 50 μM | | |
|---|---|---|---|---|---|---|
| # | d (Å) | ρ (el./Å³) | σ (Å) | d (Å) | ρ (el./Å³) | σ (Å) |
| 1 | 11.67^{±0.02} | 0.4245^{±0.0002} | 2.915^{±0.004} | 10.7^{±0.1} | 0.447^{±0.002} | 3.05^{±0.02} |
| 2 | 9.1^{±0.2} | 0.3415^{±0.0002} | 2.915^{±0.004} | 10.8^{±0.2} | 0.3399^{±0.0002} | 3.05^{±0.02} |
| Slab | Tb³⁺ 100 μM | | | Tb³⁺ 200 μM | | |
| # | d (Å) | ρ (el./Å³) | σ (Å) | d (Å) | ρ (el./Å³) | σ (Å) |
| 1 | 10.65^{±0.01} | 0.474^{±0.002} | 3.26^{±0.04} | 11.22^{±0.06} | 0.464^{±0.001} | 3.11^{±0.02} |
| 2 | 10.2^{±0.2} | 0.3402^{±0.0002} | 3.26^{±0.04} | 10.6^{±0.2} | 0.3399^{±0.0002} | 3.11^{±0.02} |

TABLE S7
Fitting parameters from XR measurements from adsorbed layers
of solutions containing either LBT1-LLA/La³⁺, or LBT1-3/La³⁺
at bulk concentrations of 100 μM of peptide and 400 μM of La³⁺.

| Slab | LBT1-LLA 100 μM/La³⁺ 400 μM | | | LBT1-3 100 μM/La³⁺ 400 μM | | |
|---|---|---|---|---|---|---|
| # | d (Å) | ρ (el./Å³) | σ (Å) | d (Å) | ρ (el./Å³) | σ (Å) |
| 1 | 13.30^{±0.07} | 0.4952^{±0.0006} | 3.584^{±0.007} | 13.31^{±0.02} | 0.4550^{±0.0002} | 2.913^{±0.004} |
| 2 | 9.95^{±0.05} | 0.4062^{±0.0005} | | 9.4^{±0.1} | 0.3427^{±0.0002} | 2.913^{±0.004} |
| 3 | 8.2^{±0.1} | 0.3464^{±0.0006} | | — | — | — |

TABLE S8
Fitting parameters from XR measurements from adsorbed layers of solutions
containing of LBT1-LLA/Tb³⁺/La³⁺ at a fixed peptide bulk concentration
of 100 μM and different equimolar concentrations of Tb³⁺ and La³⁺.

| Slab | Tb³⁺ 25 μM/La³⁺ 25 μM | | | Tb³⁺ 50 μM/La³⁺ 50 μM | | |
|---|---|---|---|---|---|---|
| # | d (Å) | ρ (el./Å³) | σ (Å) | d (Å) | ρ (el./Å³) | σ (Å) |
| 1 | 12.57^{±0.04} | 0.4482^{±0.0004} | 3.123^{±0.006} | 12.71^{±0.04} | 0.4728^{±0.0004} | 3.248^{±0.005} |
| 2 | 8.26^{±0.05} | 0.3579^{±0.0003} | 3.123^{±0.006} | 8.57^{±0.03} | 0.3677^{±0.0003} | 3.248^{±0.005} |
| Slab | Tb³⁺ 100 μM/La³⁺ 100 μM | | | Tb³⁺ 150 μM/La³⁺ 150 μM | | |
| # | d (Å) | ρ (el./Å³) | σ (Å) | d (Å) | ρ (el./Å³) | σ (Å) |
| 1 | 12.7^{±0.1} | 0.492^{±0.001} | 3.30^{±0.01} | 12.4^{±0.1} | 0.498^{±0.001} | 3.38^{±0.01} |
| 2 | 8.72^{±0.08} | 0.391^{±0.001} | 3.30^{±0.01} | 8.92^{±0.06} | 0.396^{±0.001} | 3.38^{±0.01} |
| 3 | 9.2^{±0.3} | 0.3404^{±0.0006} | 3.30^{±0.01} | 9.5^{±0.2} | 0.3418^{±0.0005} | 3.38^{±0.01} |
| Slab | Tb³⁺ 200 μM/La³⁺ 200 μM | | | Tb³⁺ 350 μM/La³⁺ 350 μM | | |
| # | d (Å) | ρ (el./Å³) | σ (Å) | d (Å) | ρ (el./Å³) | σ (Å) |
| 1 | 12.63^{±0.09} | 0.4952^{±0.0009} | 3.52^{±0.01} | 12.4^{±0.2} | 0.502^{±0.001} | 3.59^{±0.01} |
| 2 | 9.28^{±0.05} | 0.3968^{±0.0007} | 3.52^{±0.01} | 9.53^{±0.06} | 0.403^{±0.001} | 3.59^{±0.01} |
| 3 | 8.7^{±0.2} | 0.3418^{±0.0004} | 3.52^{±0.01} | 8.1^{±0.2} | 0.3452^{±0.0009} | 3.59^{±0.01} |

| | |
|---|---|
| Slab | Tb³⁺ 500 μM/La³⁺ 500 μM |

| # | d (Å) | ρ (el./Å³) | σ (Å) |
|---|---|---|---|
| 1 | 12.2^{±0.2} | 0.502^{±0.002} | 3.60^{±0.02} |
| 2 | 9.5^{±0.1} | 0.401^{±0.001} | 3.60^{±0.02} |
| 3 | 7.1^{±0.2} | 0.346^{±0.001} | 3.60^{±0.02} |

TABLE S9
Fitting parameters from XR measurements from adsorbed layers of solutions
containing of LBT1-3/Tb³⁺/La³⁺ at a fixed peptide bulk concentration
of 100 μM and different equimolar concentrations of Tb³⁺ and La³⁺.

| Slab | Tb³⁺ 25 μM/La³⁺ 25 μM | | | Tb³⁺ 50 M/La³⁺ 50 μM | | |
|---|---|---|---|---|---|---|
| # | d (Å) | ρ (el./Å³) | σ (Å) | d (Å) | ρ (el./Å³) | σ (Å) |
| 1 | 11.37^{±0.08} | 0.450^{±0.001} | 3.16^{±0.02} | 12.10^{±0.07} | 0.445^{±0.001} | 3.16^{±0.02} |
| 2 | 9.9^{±0.2} | 0.3404^{±0.0003} | | 11.8^{±0.3} | 0.3338^{±0.0002} | |
| Slab | Tb³⁺ 100 μM/La³⁺ 100 μM | | | Tb³⁺ 150 μM/La³⁺ 150 μM | | |
| # | d (Å) | ρ (el./Å³) | σ (Å) | d (Å) | ρ (el./Å³) | σ (Å) |
| 1 | 11.60^{±0.05} | 0.4574^{±0.0007} | 3.02^{±0.01} | 11.80^{±0.04} | 0.4576^{±0.0006} | 3.06^{±0.01} |
| 2 | 10.1^{±0.3} | 0.3393^{±0.0002} | | 10.5^{±0.3} | 0.3390^{±0.0002} | |
| Slab | Tb³⁺ 200 μM/La³⁺ 200 μM | | | Tb³⁺ 350 μM/La³⁺ 350 μM | | |
| # | d (Å) | ρ (el./Å³) | σ (Å) | d (Å) | ρ (el./Å³) | σ (Å) |
| 1 | 13.05^{±0.05} | 0.4317^{±0.0007} | 3.60^{±0.01} | 12.36^{±0.03} | 0.4613^{±0.0006} | 3.339^{±0.009} |
| 2 | 14.9^{±0.1} | 0.3342^{±0.0001} | | 14.9^{±0.2} | 0.3397^{±0.0001} | |

| | |
|---|---|
| Slab | Tb³⁺ 500 μM/La³⁺ 500 μM |

| # | d (Å) | ρ (el./Å³) | σ (Å) |
|---|---|---|---|
| 1 | 12.14^{±0.07} | 0.457^{±0.001} | 2.82^{±0.02} |
| 2 | 12.9^{±0.3} | 0.3378^{±0.0002} | |

The following Aspects are illustrative only and do not limit the scope of the present disclosure or the appended claims. It should be understood that the present technology can include any combination of any part or parts of any one or more of the following Aspects.

Aspect 1. A method, comprising: complexing a peptide-comprising surfactant (PEPS) to a rare earth element (REE) cation in a solution so as to form a PEPS-REE complex, the PEPS comprising a REE-binding region that preferentially binds to one or more REEs; and optionally recovering the REE cation.

The solution can include REE cations and non-REE cations. The solution can also include, for example, two or more types of REE cations.

Recovering can be accomplished by, effecting release of the cation from the binding loop; this can be accomplished with a stripping agent. Commonly used such agents can include, for example, include weak acid solutions (HCl) or structures that contain carboxylates like citrate; citric acid can also be used. Without being bound to any particular theory or embodiment, these can operate by either protonating the carboxylate groups (HCl) of the coordinating LBT sidechains or providing alternative carboxylate ligands to replace them (sodium citrate); citric acid can do both. It should be understood, of course, that the foregoing examples are not exhaustive.

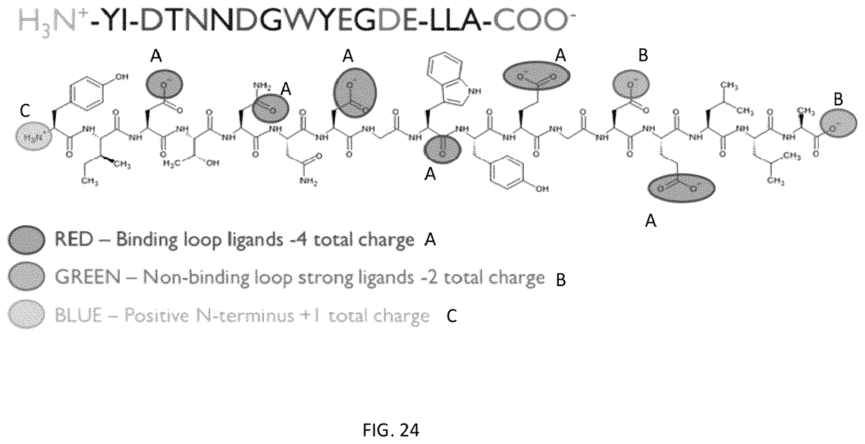

One can make modifications to a PEPS's structure to alter charge on the PEPS while also preserving the PEPS's ability to bind selectively between lanthanide cations. FIG. 24 provides the structure of a PEPS we refer to as LBT1-5 to note that the peptide has a net charge of −5 in the unbound state.

Shown in red (and labeled with A) are the coordinating ligands on this structure, green (label B) indicates non-coordinating negatively charged ligands, and blue (label C) is the positively charged N terminus. The free energy of binding of this peptide as measured by plate reader in our study is shown in the adjacent figure. La3+ is bound weakly, Tb³⁺ is bound most strongly, and there is a weak upturn in selectivity for lanthanides that are smaller and heavier than Tb^{3+.}

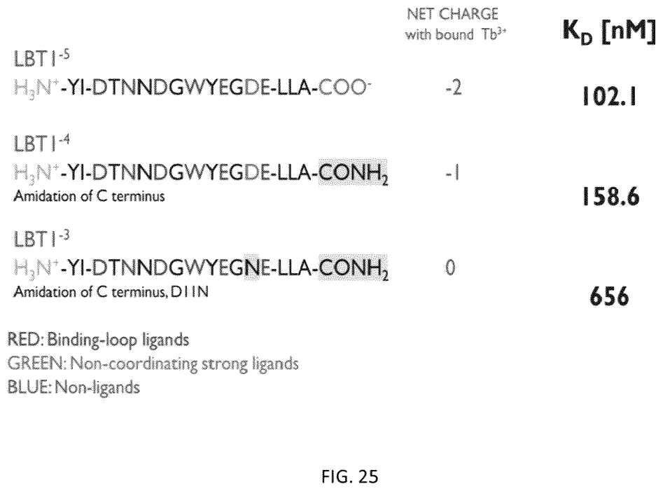

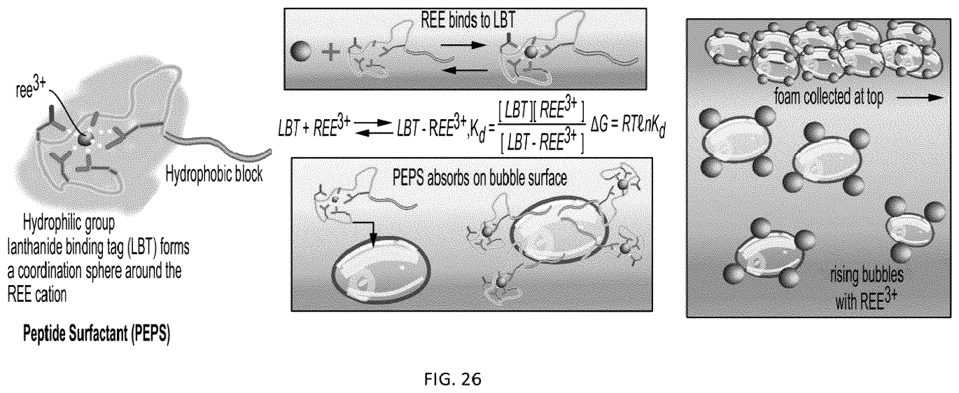

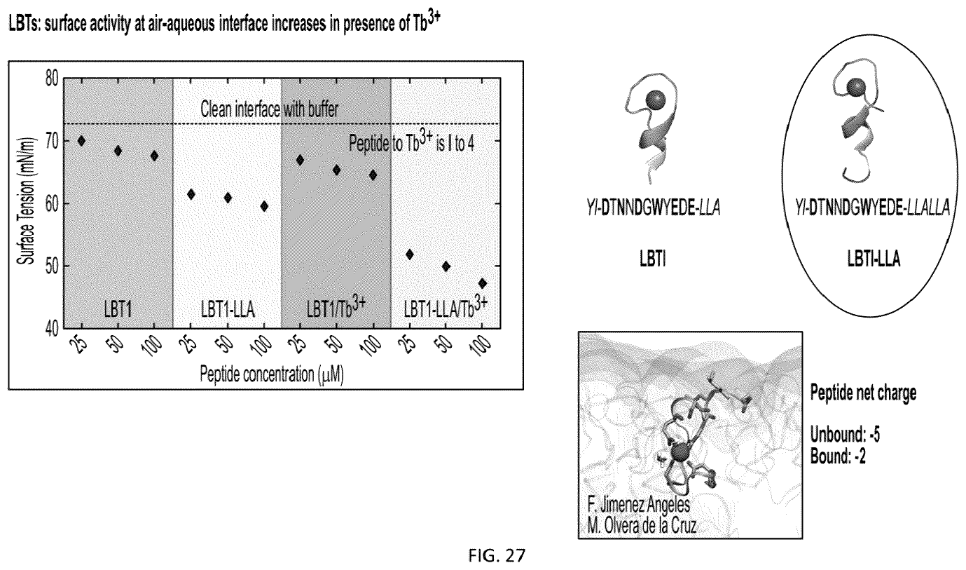

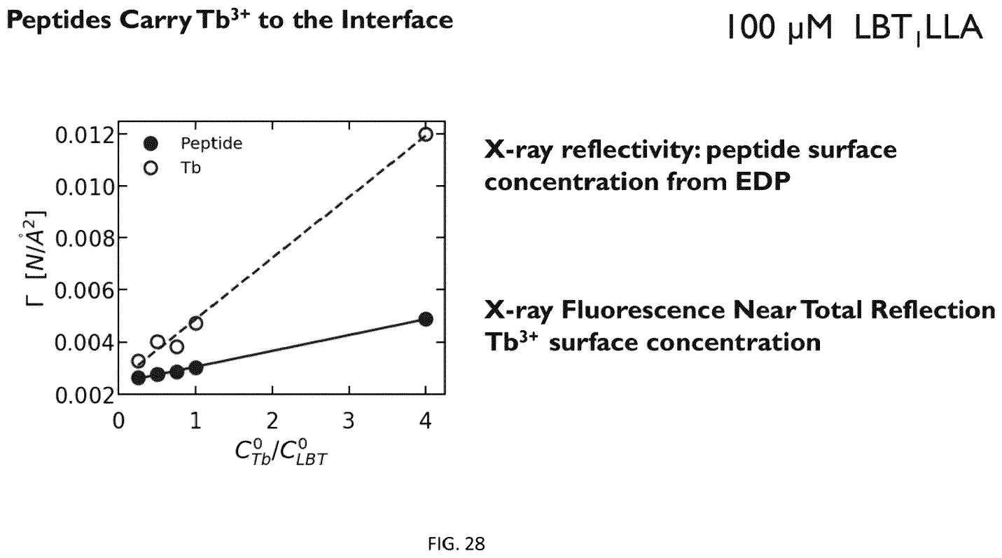

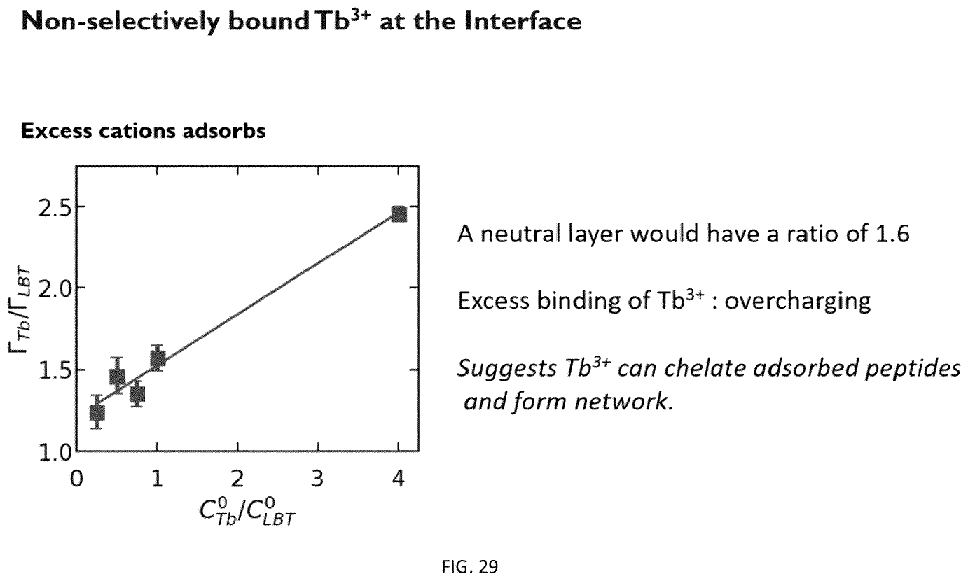

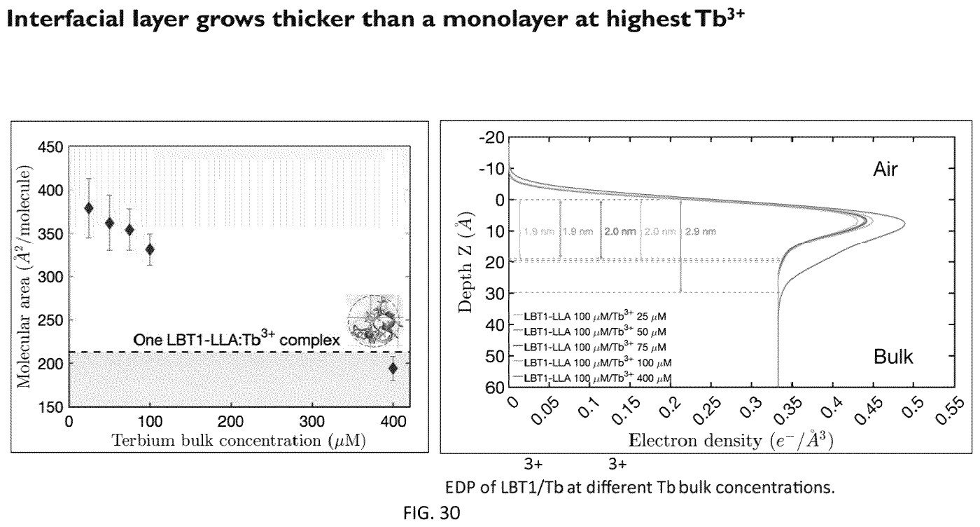

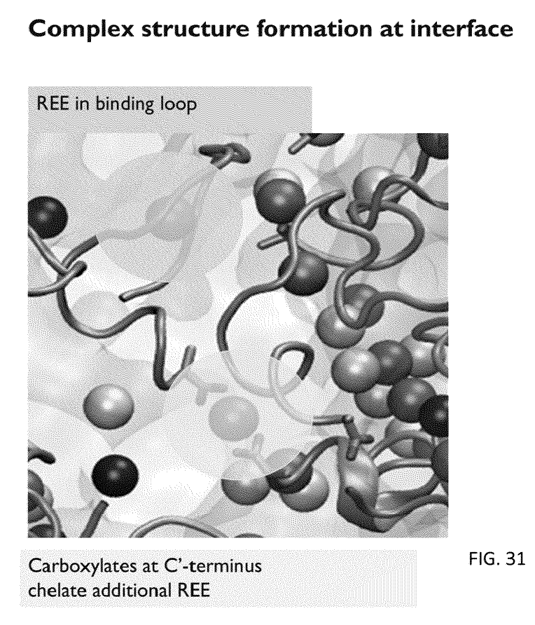

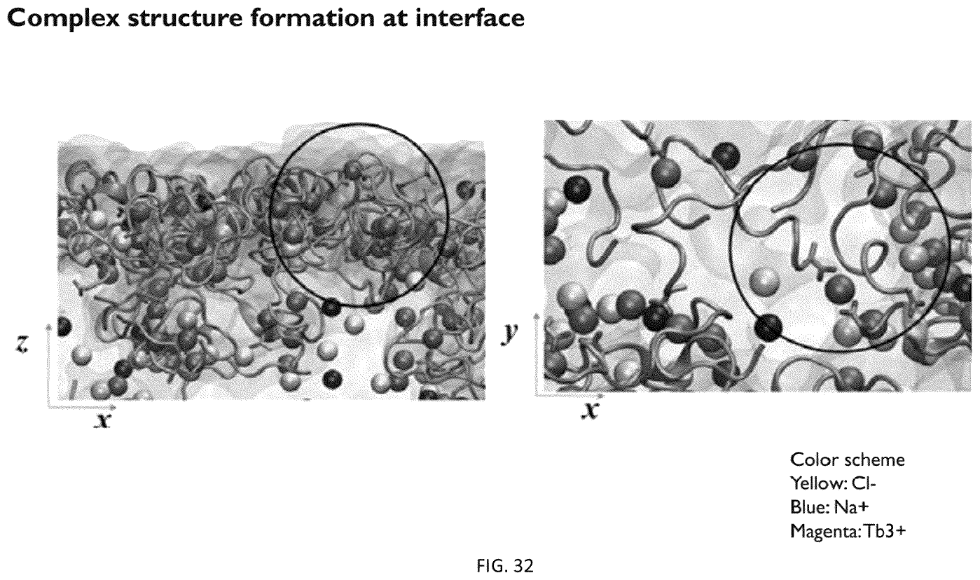

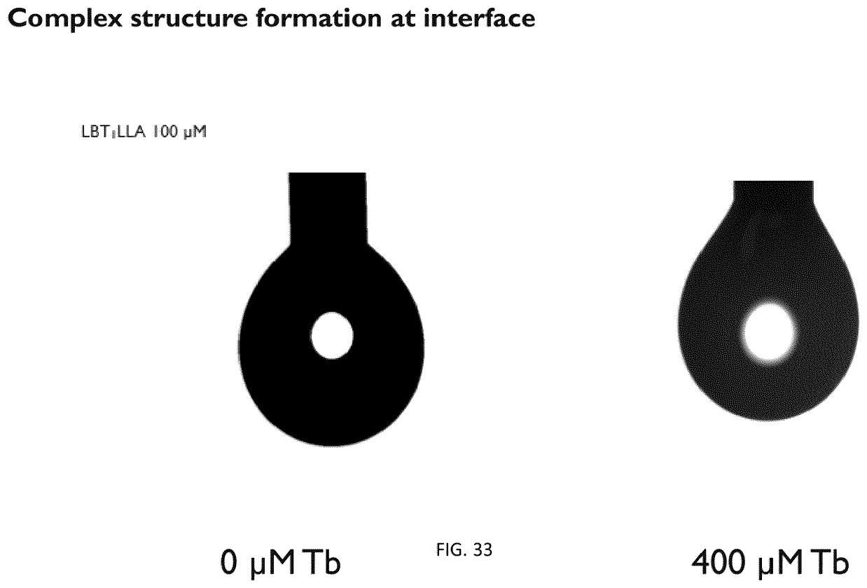

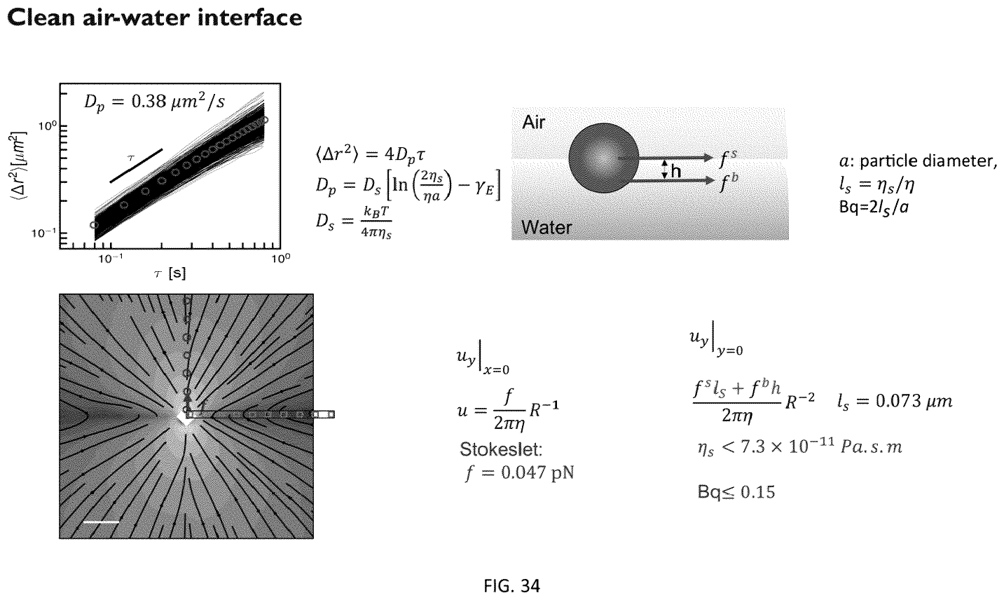

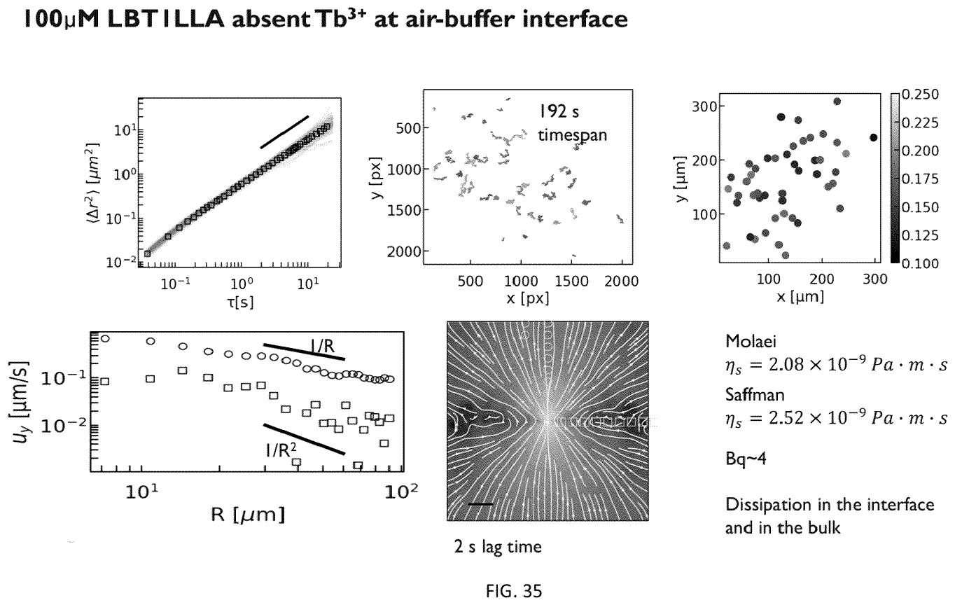

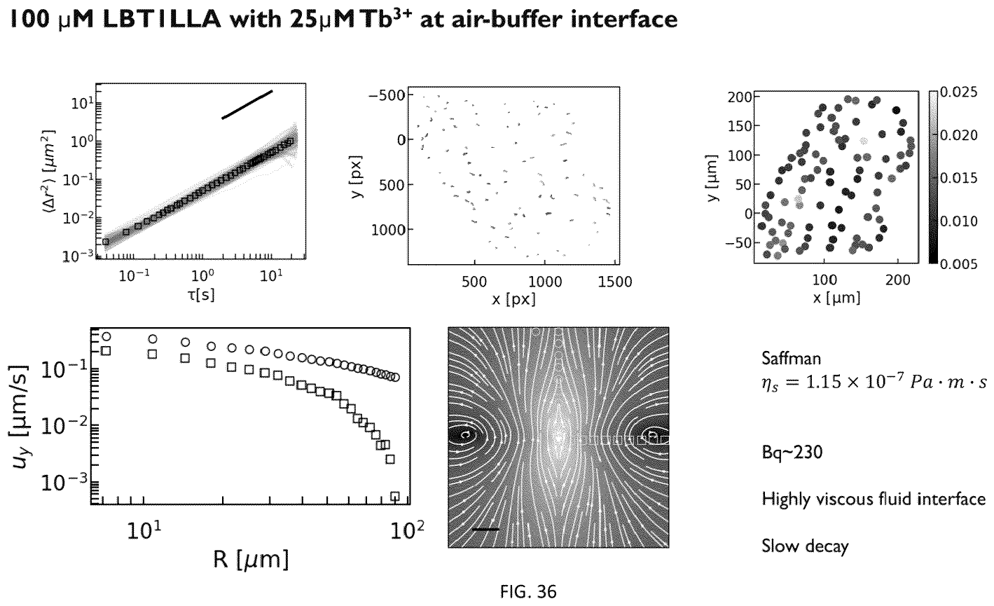

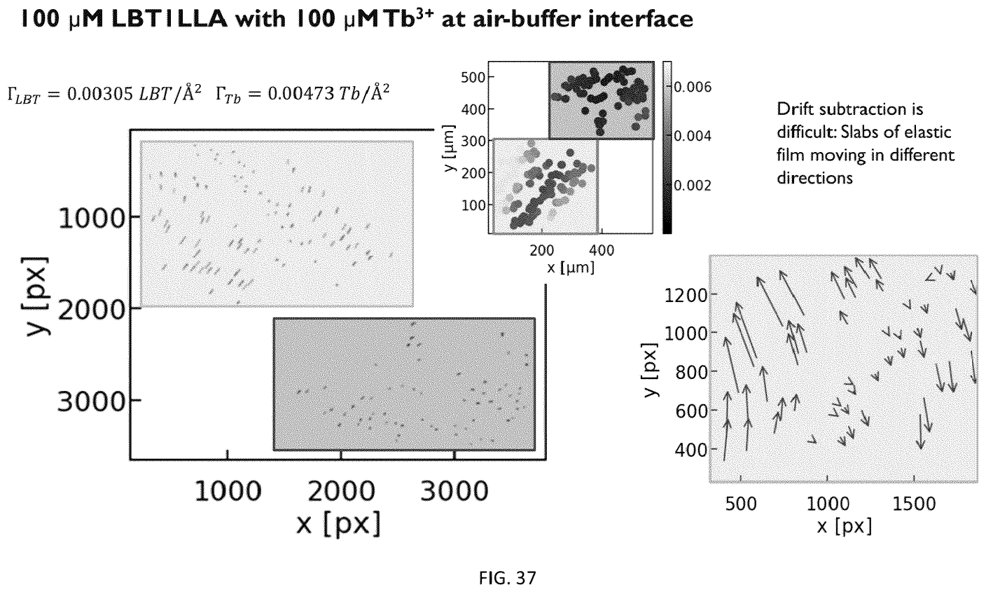

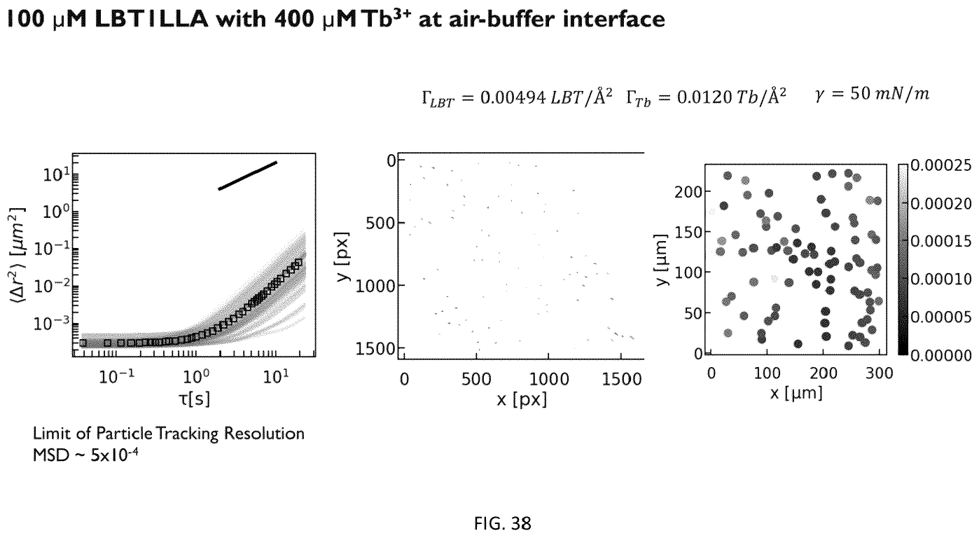

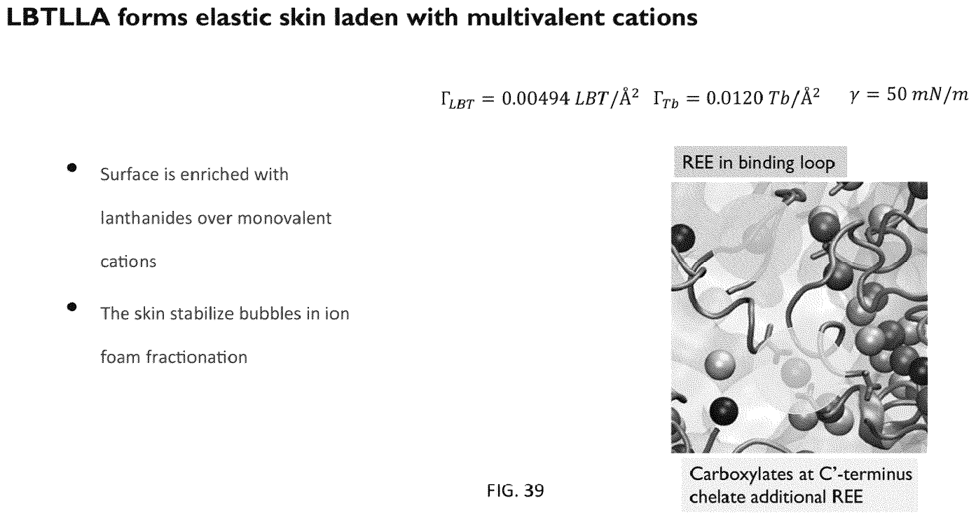

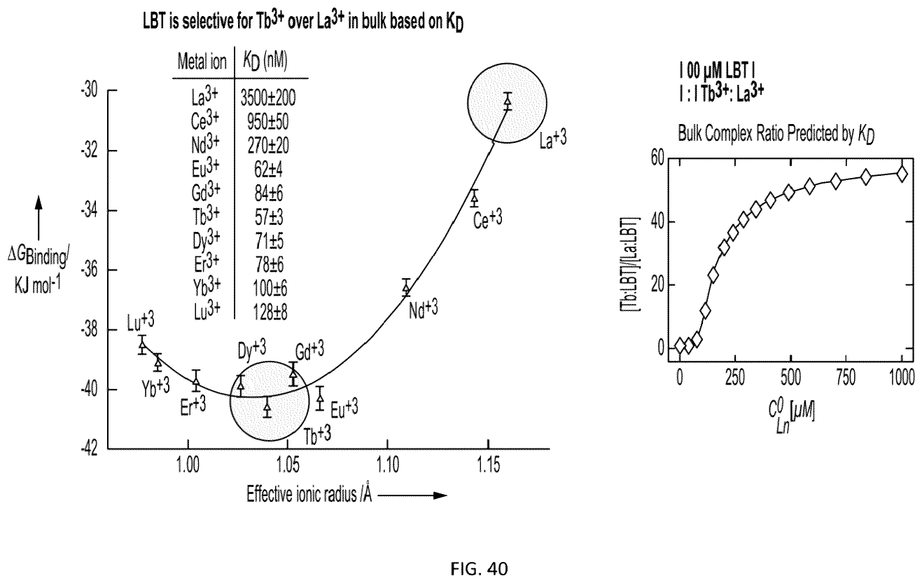

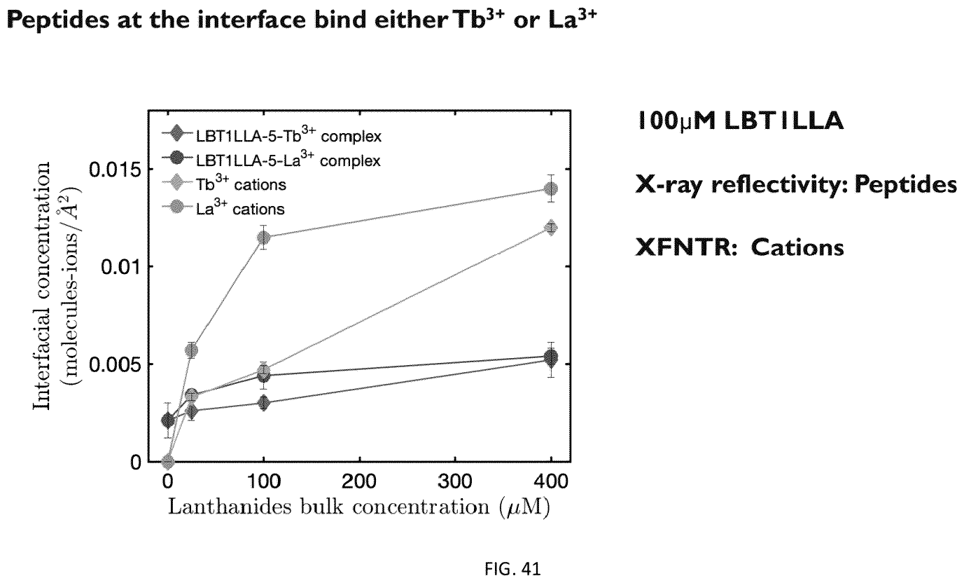

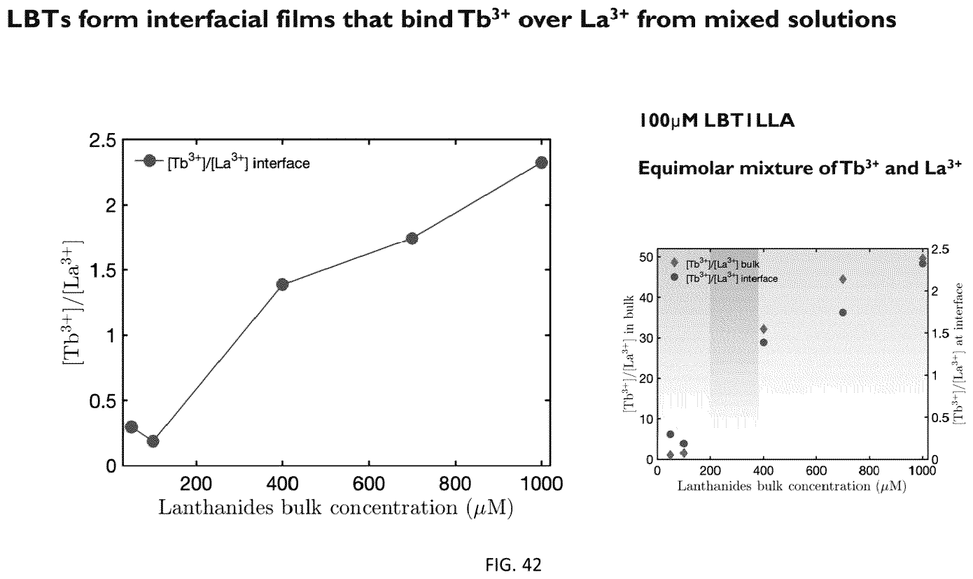

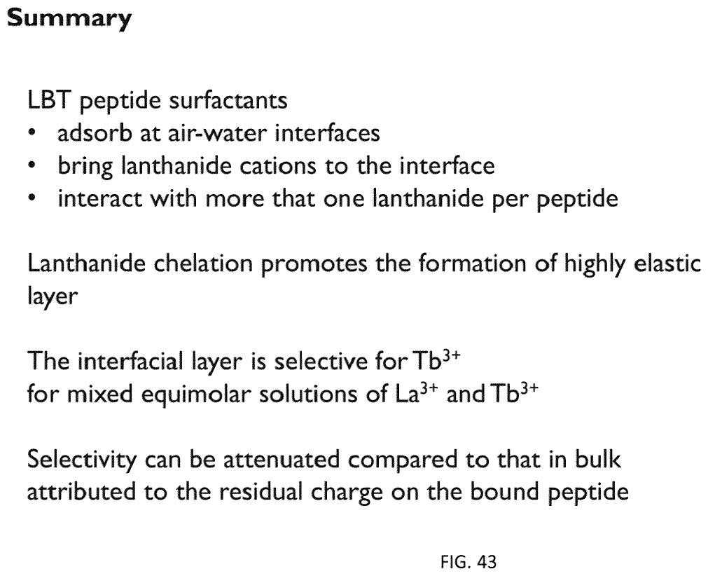

Three variants of the PEPS sequence (FIG. 25) were adjusted to preserve the binding selectivity among the lanthanides by amending non-binding ligands outside and inside of the loop.

For example, for LBT1-4, we reduced the charge on the cation by amidation of the C terminus. On LBT1-3, we reduced the charge further by replacing the non-coordinating charged residue D11 with the polar but uncharged N. It should thus accordingly be understood that a user can replace or modify a non-coordinating residue.

While these changes reduce the affinity of the PEPS for the cation, they preserve the selectivity among them of the parent structure, as is apparent in the free energy of binding for the LBT1-3 peptide.

Aspect 2. The method of Aspect 1, wherein the REE-binding region comprises a lanthanide binding tag (LBT or LBT tag). An LBT can be a peptide sequence designed to mimic and enhance the lanthanide binding ability of the naturally occurring EF hand structural motif that is present in lanthanide-binding proteins (for example, lanmodulin) and calcium binding proteins (for example, calmodulin).

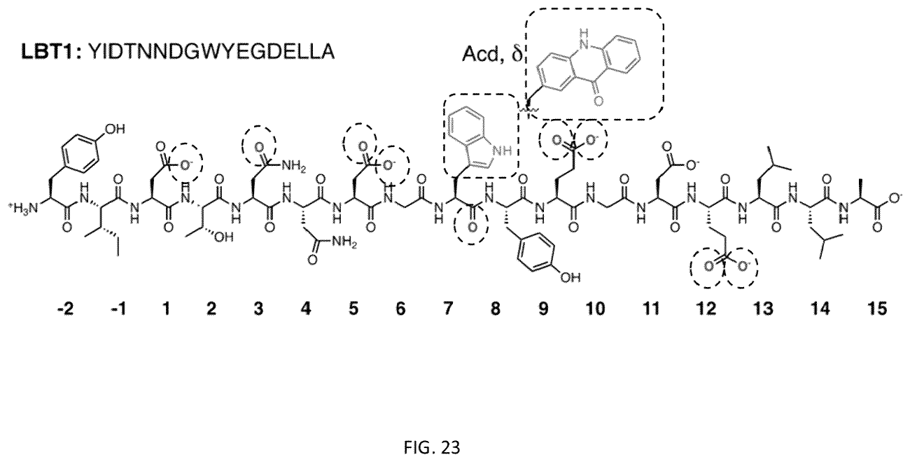

Atoms of an LBT that coordinate the cation in the parent peptide LBT1 are highlighted in FIG. 23 with dashed circles The tryptophan (Trp) or acridonylalanine (Acd) amino acid used to photo-sensitize the bound lanthanide (Trp: Tb³⁺, Acd: Eu³⁺) are highlighted with dashed rectangles. LBTs are designed to retain or enhance the selectivity for cations by modifying the binding amino acids within the loop and adding flanking peptides outside of the loop to stabilize the loop binding structure.

The exemplary table below shows example sequences characterized for terbium binding. This table shows that binding can be retained and/or enhanced through a wide variety of mutations, including fusion of the LBT terminus to the protein SUMO (Small Ubiquitin-like Modifier).

| | Sequence | Modification | C-Terminus | Charge |
|---|---|---|---|---|
| LBT1 | YIDTNNDGWYEGDELLA | | Acid | −5 |
| LBT1(−5)-Acd | YIDTNNDGδYEGDELLA | W7Acd | Acid | −5 |
| LBT1(−3)-Acd | YIDTNNDGδYEGNELLA | W7Acd D11N | Amide | −3 |
| LBT1(−4) | YIDTNNDGWYEGDELLA | | Amide | −4 |
| LBT1LLA(−5) | YIDTNNDGWYEGDELLALLA | | Acid | −5 |
| LBT1LLA(−4) | YIDTNNDGWYEGDELLALLA | | Amide | −4 |
| SUMO-LBT1 | YIDTNNDGWYEGDELLA | N-term. SUMO | Acid | (−5) |
| | YIETNNDGWYEGDELLA | D1E | Amide | −4 |
| | YINTNNDGWYEGDELLA | D1N | Amide | −3 |
| | YIDTENDGWYEGDELLA | N3D | Amide | −5 |
| | YIDTENDGWYEGDELLA | N3E | Amide | −5 |
| | YIDTQNDGWYEGDELLA | N3Q | Amide | −4 |
| | YIDTNNEGWYEGDELLA | D5E | Amide | −4 |
| | YIDTNNNGWYEGDELLA | D5N | Amide | −3 |
| | YIDTNNDGWYQGDELLA | E9Q | Amide | −3 |
| | YIDTNNDGWYDGDELLA | E9D | Amide | −4 |
| | YIDTNNDGWYTGDELLA | E9T | Amide | −3 |
| | YIDTNNDGWYSGDELLA | E9S | Amide | −3 |
| | YIDTNNDGWYEGDDLLA | E12D | Amide | −4 |
| | YIETDNDGWYEGDELLA | N3D D1E | Amide | −5 |
| | YINTDNDGWYEGDELLA | N3D DIN | Amide | −4 |
| | YIDTDNEGWYEGDELLA | N3D D5E | Amide | −5 |
| | YIDTDNNGWYEGDELLA | N3D D5N | Amide | −4 |
| | YIDTDNDGWYQGDELLA | N3D E9Q | Amide | −4 |
| | YIDTDNDGWYDGDELLA | N3D E9D | Amide | −5 |
| | YIDTDNDGWYTGDELLA | N3D E9T | Amide | −4 |
| | YIDTDNDGWYSGDELLA | N3D E9S | Amide | −4 |
| | YIDTDNDGWYEGDDLLA | N3D E12D | Amide | −5 |
| | YIDTDNDGWYEGNELLA | D11N | Amide | −3 |
| | YIDTDNDGWYEGNELLA | N3D D11N | Amide | −4 |
| | YIETNNDGWYEGNELLA | D11N D1E | Amide | −3 |
| | YINTNNDGWYEGNELLA | D11N DIN | Amide | −2 |
| | YIDTENDGWYEGNELLA | D11N N3E | Amide | −4 |
| | YIDTQNDGWYEGNELLA | D11N N3Q | Amide | −3 |
| | YIDTNNEGWYEGNELLA | D11N D5E | Amide | −3 |
| | YIDTNNNGWYEGNELLA | D11N D5N | Amide | −2 |
| | YIDTNNDGWYQGNELLA | D11N E9Q | Amide | −2 |
| | YIDTNNDGWYDGNELLA | D11N E9D | Amide | −3 |
| | YIDTNNDGWYTGNELLA | D11N E9T | Amide | −2 |
| | YIDTNNDGWYSGNELLA | D11N E9S | Amide | −2 |
| | YIDTNNDGWYEGNDLLA | D11N E12D | Amide | −3 |
| | CIDTNNDGWCEGDELLA | Cys2 | Amide | −4 |
| | CIDTDNDGWCEGDELLA | Cys2 N3D | Amide | −5 |
| | CIDTNNDGWCEGNELLA | Cys2 D11N | Amide | −3 |
| | YIDPDKDGWIDLKELLA | LnM EF1 | Amide | −2 |
| | YIDPDKDGWLDAKELLA | LnM EF2 | Amide | −2 |
| | YIDPDNDGWLDKKELLA | LnM EF3 | Amide | −2 |
| | YINPDNDGWIDARELLA | LnM EF4 | Amide | −2 |
| | YIDPDNDGWIDGKELLA | LnM-LBT1 | Amide | −3 |

Binding between the PEPS and the REE can be characterized by, for example, coordination number. One can define the coordination number (CN) to be the number of oxygen atoms (both charged and uncharged) from the LBT peptide that directly interact with the cation (i.e., not counting coordinated waters). A structure that has a CN as low as 6 in the bulk that has adsorbed as LBT:REE complex with selectivity for one cation over another. Without being bound to any particular theory, a CN less than 6 may not in all instances have the synergistic responses within the complex to discriminate between cations. The PEPS can have a coordination number with the REE of, for example, 4, 5, 6, 7, 8, 9, 10, 11, 12, 13, 14, or 15; the foregoing values are exemplary and are not limiting.

The CN and the spatial arrangement of the coordinated oxygens varies from cation to cation. The strength and number of these interactions work in a coordinated fashion to generate the affinity of the binding loop for the cation and the selectivity among the cations in bulk solution.

Without being bound to any particular theory, binding occurs in the bulk, followed by adsorption of LBT:REE complexes at the air/liquid interface. In some instances, the selectivity for a given REE can be higher in the bulk than at the interface, but this is not necessarily the case. The ratio of the selectivity for a given REE in the bulk to the ratio of the selectivity for that REE at the interface can be, for example, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1.9:1, 1.8:1, 1.7:1, 1.6:1, 1.5:1, 1.4:1, 1.3:1, 1.2:1, or even 1:1.

In some instances, a PEPS:REE complex can be net-negative in charge. In some instances, a PEPS:REE complex can be net-neutral in charge. In some instances, a PEPS:REE complex can be net-positive in charge. The characteristics of the PEPS can be selected to achieve any of the foregoing charge characteristics.

Aspect 3. The method of Aspect 2, wherein the PEPS further comprises at least one additional hydrophobic moiety in addition to the LBT, the at least one additional hydrophobic moiety optionally comprising at least one of a non-polar peptide, an alkyl chain, and a hydrophobic hydrocarbon-containing structure.

The amino acids that define peptide sequences are commonly divided into four groups, i.e., non-polar, polar, positively charged and negatively charged.

The non-polar peptides that can impart hydrophobicity include glycine (G), alanine (A), valine (V), cysteine (C), proline (P), leucine (L), isoleucine (I), methionine (M), tryptophan (W), phenylalanine (F). Without being bound to any particular theory or embodiment, their addition can enhance the hydrophobic nature without disrupting the binding loop.

LLA (leucine-leucine-alanine) was used as an example terminal hydrophobic moiety because it is a repeat of the terminal hydrophobic domain in the LBTs in the literature. These termini were selected because they were shown to stabilize the loop binding structure; this is thought to be related to the ability of LLA to form an alpha helix structure. As discussed elsewhere herein, we added a repeated LLA to form the enhance the surface activity without disrupting the stabilization of the loop.

Other sequences of the non-polar peptides at the termini or the binding loop can be used. Such sequences can be guided by, e.g., rational selection, molecular simulation and/or machine learning. They can be selected to provide surface activity while preserving the ability of the binding loop to interact selectively with the cation in the bulk and to retain the cation at the interface.

As described elsewhere herein, there is another location in the binding region sequence that can contribute to the hydrophobic character or the bound peptide and cation. In one sequence provided herein, the W (tryptophan) in the linear sequence, located at position 7 in the current binding loop is the so-called “antenna” position for fluorescence studies. The antenna is excited and transfers its excitation to bound cation.

In some mutants, the mutants replace the hydrophobic tryptophan with the more hydrophobic non-natural amino acid acridone at this location; acridone also acts an antenna. Tryptophan and acridone are both hydrophobic; acridone is more hydrophobic that tryptophan. Without being bound to any particular theory or embodiment, this can promote adsorption of the PEPS. It should be understood, however, that it is not a requirement to have an “antenna” in the PEPS, and one can replace the tryptophan with a different amino acid that does not act as an antenna in the manner of tryptophan.

In addition to adjusting the amino acid selection and/or sequence, one can also append alkyl chains or other hydrophobic hydrocarbon containing structures at the termini outside of the binding loop to adjust the hydrophobicity of the PEPS. As a non-limiting example, the following peptide was shown to be surface active:

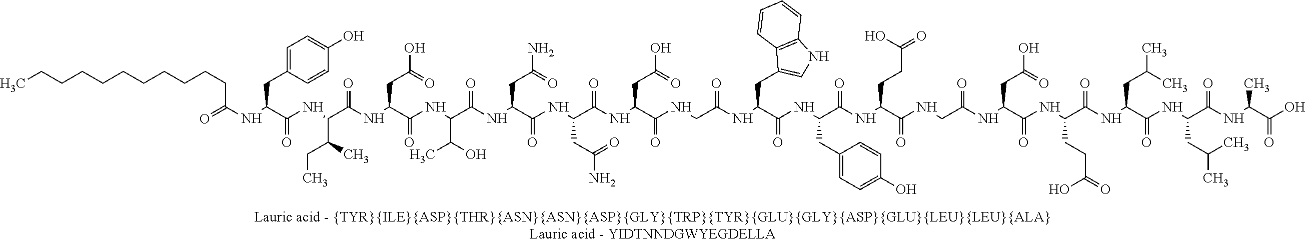 [SMILES: CCCCCCCCCCCC(=O)N[C@@H](Cc1ccc(O)cc1)C(=O)N[C@H](C(=O)N[C@@H](CC(=O)O)C(=O)NC(C(=O)N[C@@H](CC(N)=O)C(=O)N[C@@H](CC(N)=O)C(=O)N[C@@H](CC(=O)O)C(=O)NCC(=O)N[C@@H](Cc1c[nH]c2ccccc12)C(=O)N[C@@H](Cc1ccc(O)cc1)C(=O)N[C@@H](CCC(=O)O)C(=O)NCC(=O)N[C@@H](CC(=O)O)C(=O)N[C@@H](CCC(=O)O)C(=O)N[C@@H](CC(C)C)C(=O)N[C@@H](CC(C)C)C(=O)N[C@@H](C)C(=O)O)C(C)O)[C@@H](C)CC]

Other adjustments can also be made to modify the hydrophobicity of the PEPS. One binding loop can include the following polar amino acids: the negatively charged aspartic acid D, the polar asparagine N, negatively charged glutamic acid E. In this example, within the binding loop, the polar amino acids D1, N3, D5, E9, and E12 all participate in multidentate binding of the cation Tb³ in the loop. In addition, the backbone carbonyl group at the W7 participates in the binding.

Mutant PEPS can be designed using ML approaches and rational approaches in which hydrophilic peptides with either charged or polar moieties that participate in the multi-dentate binding of the loop domain and the cation are swapped to change selectivity in the bulk and to retain the cation at the interface. In performing such mutations at one or more locations on the peptide, one can exploit the polar uncharged amino acids serine (S), thrionine (T), tyrosine (Y), asparagine (N) and glutamine Q_{c} and the charged amino acids, the positively charged amino acids lysine (K), arginine (R) and histidine (H) and the negatively charged amino acids aspartic acid (D) and glutamic acid (E).

One can also perform PEPS mutations of non-coordinating ligands to manage excess charge on binding. Without being bound to any particular theory or embodiment, one can create mutants to reduce the non-selective binding that one can hypothesize is caused by the charge of -2 on the peptide that is bound to a single Tb cation. This approach can include swapping of charged and uncharged polar peptides that do not directly participate in coordination of the cation, e.g., replacing the D11 with an N₁₁.

One can also change the C-terminus to reduce negative charge on peptide. Peptides typically have an N terminus that is positively charged and a C terminus (a terminal carboxy group) that is negatively charged. The negatively charged C terminus is predicted in simulation to participate in network formation at the interface. To reduce this network formation, one can replace the terminal carboxy group, for example, with a terminal —CONH₂. One can also replace the terminal carboxy group with other groups that reduce the negative charge of the peptide.

Further (and again without being bound to any particular theory or embodiment), the introduction of spacers between the binding loop and the hydrophobe can protect the binding loop from the environment of the interface. For example, repeated ethoxy groups (shown below) can be inserted between the binding loop and the terminal hydrophobic groups to increase the distance between the interface and the binding loop domain.

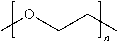

It should be understood, however, that ethoxy groups are illustrative only, as the spacer is not limited to ethoxy groups, and other spacers can be used instead or even with ethoxy groups.

One can modify LBT structures to promote adsorption at the interface. A trypotophan residue is hydrophobic, and can also confer surface activity to the native structure. Additional hydrophobes can further promote adsorption to the fluid interface or can be designed to promote self-assembly at the interface or in solution. The hydrophobes can be grafted onto the PEPS structure like alkyl chains or other hydrocarbon structures that can be appended to selected sites on the LBT. Alternatively, the hydrophobes can be amino acid residues with hydrophobic side chains strategically added to locations within the peptide sequence.

Hydrophobe addition may not be necessary if the hydrophobic:hydrophilic balance of the PEPS is too hydrophobic, for example, causing the PEPS lose their solubility in water. In that case, one can add amino acid residues that are charged; one can do so such that the overall complex structure is neutral or positive.

Aspect 4. The method of Aspect 3, wherein (a) at least one hydrophobic moiety is disposed at an end of the PEPS, (b) the PEPS comprises a spacer, the spacer optionally comprising an ethoxy group, disposed between the at least one hydrophobic moiety and the REE-binding region of the PEPS, or both (a) and (b).

Aspect 5. The method of any one of Aspects 1-4, wherein the PEPS selectively binds a first REE over a second REE. It should be understood, however, that the disclosed technology can be used to separate REEs from one another that are comparatively close in size. The disclosed technology can be used to separate a given REE from all other REEs that may be present in a sample.

Aspect 6. The method of Aspect 5, wherein the first REE is Tb and the second REE is La. Other exemplary REEs include Pr, Nd, Dy, and Ce.

Aspect 7. The method of any one of Aspects 1-6, further comprising effecting disposition of the PEPS-REE complex to an air/liquid interface. Such an interface can be an air/water interface or other air/aqueous interface.

Aspect 8. The method of Aspect 7, wherein the air/water interface is present at a bubble.

Aspect 9. The method of any one of Aspects 1-8, wherein the method is performed so as to give rise to an aggregate comprising a plurality of PEPS-REE complexes.

Aspect 10. The method of Aspect 9, wherein the aggregate comprises from 1 to 50 PEPS-REE complexes.

Aspect 11. The method of any one of Aspects 1-8, wherein the method is performed so as to give rise to a film comprising a plurality of PEPS-REE complexes at an air/liquid interface. Such an interface can be an air/water interface or other air/aqueous interface.

Aspect 12. The method of Aspect 11, wherein the film is thicker than a monolayer.

Aspect 13. The method of any one of Aspects 1-12, further comprising sparging bubbles through the solution under such conditions that the PEPS-REE cation complex adsorbs to a bubble; and recovering the REE cation.

Aspect 14. The method of any one of Aspects 1-13, wherein the PEPS is designed using at least one of artificial intelligence and a genetic algorithm.

Without being bound to any particular theory or embodiment, a non-limiting computational peptide design scheme can include the following aspects:
(1) Perform all-atom molecular dynamics with enhanced sampling techniques (e.g., metadynamics) of an example (e.g., LBT-Ln³⁺) binding complex. This can include calculating key physiochemical quantities and thermodynamic properties (e.g., conformational free energy landscape, binding coordination, hydration properties) from a molecular simulation.
(2) Train a supervised learning model (such as a Deep Neural Network, for example) to build quantitative predictive correlation between MD simulation features and experimental binding affinity (AG).
(3) Apply genetic algorithm and/or ML predictions to achieve high-throughput screening for LBT mutants.

Aspect 15. The method of Aspect 14, wherein the artificial intelligence comprises machine learning.

Aspect 16. The method of any one of Aspects 14-15, wherein the PEPS is designed to preferentially bind one REE over at least one other REE.

Aspect 17. A composition, comprising: a complex comprising (i) a peptide-comprising surfactant (PEPS), the PEPS comprising at least one lanthanide binding tag (LBT) binding region, the at least one LBT comprising one or more residues arranged in a binding region that coordinates with an REE cation so as to form a PEPS-REE cation complex, and (ii) an REE cation, the REE cation of the complex coordinated with the binding region of the PEPS.

The composition can be a liquid; the composition can also be a bubble or even a foam. The PEPS-REE cation complex can be situated at an air/liquid interface. Thus a composition according to the present disclosure can be an air/liquid interface having disposed therealong at least one PEPS-REE cation complex. The interface can be an air/water interface; the interface can also be another air/aqueous interface.

As described elsewhere herein, the composition can be a bubble or even a foam. The composition can be, for example, an amount of liquid having disposed therein a plurality of bubbles, the bubbles having PEPS-REE complexes disposed along their boundaries, i.e., at the interface between air and liquid that defines the bubble.

Also as described elsewhere herein, the PEPS can include any one or more of (i) one or more hydrophobic moieties at an end of the PEPS, (ii) a spacer (which can comprise repeat units) disposed between the LBT and an end of the PEPS, e.g., between the LBT and one or more hydrophobic moieties disposed at an end of the PEPS, or both (i) and (ii).

Aspect 18. The composition of Aspect 17, wherein the composition comprises an aggregate comprising a plurality of PEPS-REE complexes.

Aspect 19. The composition of Aspect 17, wherein the composition comprises a film comprising a plurality of PEPS-REE complexes.

Aspect 20. The composition of any one of Aspects 17-19, wherein the PEPS is designed using at least one of artificial intelligence and a genetic algorithm.

Aspect 21. The composition of Aspect 20, wherein the artificial intelligence comprises machine learning.

Aspect 22. The composition of any one of Aspects 20-21, wherein the PEPS is designed to preferentially bind one REE over at least one other REE. As described herein, a PEPS can preferentially bind one REE over at least one other REE

A composition according to the present disclosure can include, for example, a first population of complexes that comprise PEPS₁:REE₁ and a second population of complexes that comprise PEPS₂:REE₂, wherein PEPS₁ differs from PEPS₂ and REE₁ differs from REE₂. The first population can be more numerous than the second population; alternatively, the second population can be more numerous than the first. The first and second populations can reside at a liquid/air interface, for example, at an air/water interface. Such an interface can be comprised in a bubble; the interface can also be comprised in a foam.

Similarly, the disclosed methods can include contacting a first PEPS and a second PEPS (wherein the first PEPS differs from the second PEPS) to a liquid that contains multiple REEs, one of which REEs can bind preferentially to the first PEPS as compared to the second PEPS, and another of which REEs can bind preferentially to the second PEPS as compared to the first PEPS). PEPS:REE complexes can then form, with the complexes then arriving at an air interface with the liquid. The REEs of the complexes can then, as described elsewhere herein, be recovered.

Aspect 23. A method, comprising application of at least one of molecular dynamics, artificial intelligence, and a genetic algorithm to design a PEPS that comprises an LBT and preferentially binds one REE over at least one other REE.

Aspect 24. The method of Aspect 23, further comprising training a supervising learning model to provide a quantitative predictive correlation between a molecular dynamics simulation features and an experimental binding affinity (AG).

Aspect 25. The method of any one of Aspects 23-24, further comprising application of a genetic algorithm and machine learning predictions to screen a plurality of candidate PEPS mutants that comprise an LBT.

As explained here, using LBTs to capture REEs provides a number of advantages. Some of these advantages include:
(i) The ability to use green surfactants to capture REEs at the air-water interface by binding the REE cations from a bath of non-REE cations. This can be dependent on binding strength, which must be high enough to capture cations from a bath of a given cation concentration. The threshold binding affinity KD should be lower than the concentration of cations in solution. KD is derived from the free energy of binding and is related to CN.
(ii) The ability to bind LBT:REE in the bulk with selectivity for one REE cation over another. Depending on the concentration of cations in the separation medium, one can sacrifice binding strength if one retains selectivity.
(iii) The ability to bind LBT:REE in the bulk with selectivity for one REE cation over another, and to retain that selectivity at the interface.

What is claimed:

1. A method, comprising:
complexing a peptide-comprising surfactant (PEPS) to a rare earth element (REE) cation in a solution so as to form a PEPS-REE complex,
the PEPS comprising a REE-binding region that preferentially binds to one or more REEs; and
optionally recovering the REE cation.

2. The method of claim 1, wherein the REE-binding region comprises a lanthanide binding tag (LBT).

3. The method of claim 2, wherein the PEPS further comprises at least one additional hydrophobic moiety in addition to the LBT, the at least one additional hydrophilic moiety optionally comprising at least one of a non-polar peptide, an alkyl chain, and a hydrophobic hydrocarbon-containing structure.

4. The method of claim 3, wherein (a) at least one hydrophobic moiety is disposed at an end of the PEPS, (b) the PEPS comprises a spacer, the spacer optionally comprising an ethoxy group, disposed between the at least one hydrophobic moiety and the REE-binding region of the PEPS.

5. The method of claim 1, wherein the PEPS selectively binds a first REE over a second REE.

6. The method of claim 1, further comprising effecting disposition of the PEPS-REE complex to an air/liquid interface.

7. The method of claim 7, wherein the air/liquid interface is present at a bubble.

8. The method of claim 1, wherein the method is performed so as to give rise to an aggregate comprising a plurality of PEPS-REE complexes.

9. The method of claim 1, wherein the method is performed so as to give rise to a film comprising a plurality of PEPS-REE complexes at an air/liquid interface.

10. The method of claim 1, further comprising sparging bubbles through the solution under such conditions that the PEPS-REE complex adsorbs to a bubble; and
recovering the REE cation.

11. A composition, comprising:
a complex comprising
(i) a peptide-comprising surfactant (PEPS), the PEPS comprising at least one lanthanide binding tag (LBT) binding region, the at least one LBT comprising one or more residues arranged in a binding region that coordinates with an REE cation so as to form a PEPS-REE cation complex, and
(ii) an REE cation,
the REE cation of the complex coordinated with the binding region of the PEPS.

12. The composition of claim 11, wherein the composition comprises an aggregate comprising a plurality of complexes.

13. The composition of claim 11, wherein the composition comprises a film comprising a plurality of complexes.

14. The composition of claim 11, wherein the complex is disposed at an air/liquid interface.

15. The composition of claim 14, wherein the air/liquid interface is an air/water interface.

16. The composition of claim 11, PEPS is selected to preferentially bind one REE over at least one other REE.

17. The composition of claim 11, wherein the air/liquid interface defines a bubble.

18. The composition of claim 17, wherein the bubble is disposed within a foam.

19. The composition of claim 17, wherein the bubble is disposed within a liquid.

20. The composition of claim 11, wherein the composition is characterized as being net-neutral or net-positive.
